(19) United States
(12) Patent Application Publication
(10) Pub. No.: US 2026/0159497 A1
(43) Pub. Date: Jun. 11, 2026

(54) SOLID FORMS OF A COMPLEMENT FACTOR B INHIBITOR

(71) Applicant: Novartis Pharma AG, Basel (CH)

(72) Inventors: Xingjuan CHANG, Changshu Suzhou (CN); Kassandra EMBERSON, Seattle, WA (US); Robert GOMEZ, Seattle, WA (US); Shahrokh KAZERANI, Seattle, WA (US); Bo LIU, Shanghai (CN); Kimberly MILLER, Seattle, WA (US); David Andrew POWELL, Seattle, WA (US); Tao SHENG, Seattle, WA (US); Daniel WATSON, Seattle, WA (US); Zhe ZHANG, Seattle, WA (US); Yi ZHAO, Changshu Suzhou (CN)

(21) Appl. No.: 19/129,147

(22) PCT Filed: Nov. 13, 2023
(86) PCT No.: PCT/CN2023/131294

§ 371 Date: May 12, 2025

Related U.S. Application Data
(60) Provisional application No. 63/425,206, filed on Nov. 14, 2022.

(51) Int. Cl. C07D 401/06; A61K 31/438

(52) U.S. Cl. CPC C07D 401/06; A61K 31/438

(57) ABSTRACT

The present disclosure relates to crystalline forms of (S)-4-(2, 2-difluoro-7-((5-methoxy-7-methyl-1H-indol-4-yl)methyl)-7-azaspiro[3.5]nonan-6-yl)benzoic acid and pharmaceutical compositions and uses of same.

### CLAIM OF PRIORITY

This application claims priority to U.S. Patent Application Ser. No. 63/425,206, filed on Nov. 14, 2022, the entire contents of which are hereby incorporated by reference including any figures.

### TECHNICAL FIELD

Disclosed herein are crystalline forms of (S)-4-(2,2-difluoro-7-((5-methoxy-7-methyl-1H-indol-4-yl)methyl)-7-azaspiro[3.5]nonan-6-yl)benzoic acid, and a p-toluenesulfonic acid salt of (S)-4-(2,2-difluoro-7-((5-methoxy-7-methyl-1H-indol-4-yl)methyl)-7-azaspiro[3.5]nonan-6-yl)benzoic acid.

### BACKGROUND

The complement system is a key component of the innate immunity system with two main functions of host defense against microbial pathogens and clearance of apoptotic cells. Since first discovered by Jules Bordet and Paul Ehrlich in the 1890s, more than a century of research on complement has uncovered its diverse roles in immune response, surveillance, homeostasis, and metabolism (Hajishengallis, Nat Immunol 2017 18:1288-1298; Sim, Immunobiology 2016 221 (10): 1037-1045; Ricklin, Nat Immunol 2010 11 (9): 785-797). The complement system comprises a large number of soluble proteins that are found in circulation and tissue as inactive zymogens that are activated upon serine protease cleavage. Activation of complement is tightly regulated by both plasma and membrane-bound regulators. Dysregulation of complement activity through genetic mutation, autoantibodies or chronic inflammation has been found to cause tissue damage in various pathological conditions, including autoimmune, inflammatory, neurodegenerative and in a broad range of renal diseases (Zipfel, Nat Rev Immunol 2009 9:729-749; Holers, Annu Rev Immunol 2014 32:433-459).

There are three activation pathways: the classical pathway (CP), lectin pathway (LP), and alternative pathway (AP) (Merle, Front Immunol 2015 6:262). The CP is activated by immunoglobulins (IgG and IgM) and immune complexes through binding of C1q to the Fc domain (Botto, Annu Rev Immunol 2002 205:395-406). The LP is activated by a group of proteins that bind to sugars on the surface of bacteria, for example, mannose binding lectin (MBL) (Garred, Immunol Rev 2016 274 (1): 74-97). In contrast to the other two pathways that require specific stimuli for activation, the AP maintains a low level of activation in plasma through a spontaneous hydrolysis process called “tickover” and can also be secondarily activated by the other two complement pathways (Lachmann, Adv Immunol 2009 104:115-149). The AP forms a rapidly self-amplified loop unless inactivated by factor H and factor I. The three activation pathways generate protease complexes termed “C3 convertases” (C3bBb and C4b2a) to cleave C3, and form C3bBbC3b as C5 convertase. The terminal complement pathway assembles C5b with other complement proteins to form C5b-9 membrane attach complex (MAC), which mediates lysis of pathogens or apoptotic cells (Bhakdi, Immunol Today 1991 12:318-320). Two soluble fragments of C3 and C5 cleavage products, C3a and C5a, also termed “anaphylatoxins” are potent chemo-attractants that trigger pro-inflammatory responses through their receptors (Klos, Mol Immunol 2009 46 (14): 2753-2766).

Complement overactivation and kidney deposition is observed in various chronic kidney diseases (CKDs) including atypical hemolytic uremic syndrome (aHUS), C3 glomerulopathy (C3G), IgA nephropathy (IgAN), membranous nephropathy (MN), ANCA-associated vasculitis (AAV), (focal segmental glomerulosclerosis (FSGS) and lupus nephritis (LN) (Harris, Semin Immunopathol 2018 40 (1): 125-140; Willows, Clin Med 2020 20 (2): 156-160). Pre-clinical and clinical evidence support the role of complement, especially the AP, in disease initiation and progression. Genetic defects in complement genes, such as CFH, CFI, CFHRs, CFB, C3 and MCP/CD46, have been directly linked to aHUS and C3G (Bu, J Am Soc Nephrol 2014 25 (1): 55-64; Marinozzi, J Am Soc Nephrol 2015 25:2053-2065; Xiao, Semin Thromb Hemost 2014 40 (4): 465-471). Complement activation by autoantibodies and immune complexes in the kidney lead to renal injury and contribute to disease progression in multiple glomerular diseases (Corvillo, Front Immunol 2019 10:886; Marinozzi, J Am Soc Nephrol 2017 28 (5): 1603-1613; Seikrit, N Engl J Med 2018 379 (25): 2479-2481). Recent studies provide evidence of kidney local production and activation of complement proteins in CKDs such as IgAN and diabetic kidney disease (Mühlig, Front Immunol 2020 11:1833; Zhou, Clin J Am Soc Nephrol 2021 16 (2): 213-224; Kelly Am J Nephrol 2015 41:48-56). It is believed the local production of complement and the unique microenvironment in the kidney make the organ more susceptible to complement overactivation (Thurman, Clin J Am Soc Nephrol 2020 11:1856).

Significant efforts have been directed towards the development of complement-targeted therapies. Eculizumab is a C5 monoclonal antibody that has been approved for treatment of aHUS. However, when tested in C3G, only a subset of patients who had higher level of C5b-9 (MAC) showed improvement of disease. This is likely due to the contribution of activation fragments at the C3 level upstream of the terminal pathway (Vivarelli, Semin Thromb Hemost 2014 40 (4): 472-477). Multiple therapeutic agents targeting different complement pathways are currently in development, each with advantages and limitations (Zipfel, Front Immunol 2019 10:2166; Thurman, Kidney Int 2016 90 (4): 746-752). Nevertheless, there remain needs for potent therapeutic compounds blocking both C3 and C5 levels of the complement system.

As the key enzyme of the AP, CFB provides a highly desirable target to block the central amplification loop and the terminal complement pathway. Knocking out CFB has been shown to be protective in rodent models of C3G (Pickering, Nat Genet 2002 31 (4): 424-428), MN (Luo, Front Immunol 2018 9:1433), ANCA-associated vasculitis (Xiao, Am J Pathol 2007 170 (1): 52-64), LN (Watanabe, J Immunol 2000 164 (2): 786-794), and multiple renal injury models (Thurman, Am J Physiol Renal Physiol 2012 302: F1529-F1536; Casiraghi, Am J Transplant 2017 17:2312-2325; Morigi, Sci Rep 2016 6:8445). Genetic deficiency of CFB in these models resulted in reduced proteinuria, protection from renal injury, and prolonged survival. Like many complement proteins, CFB circulates in its native form at high plasma concentration of 300-400 μg/mL. Recently, a selective CFB inhibitor, iptacopan (LNP023), has been shown to bind to active CFB (Schubart Proc Natl Acad Sci USA. 2019 116 (16): 7926-7931). In a Phase II clinical trial in C3G, iptacopan demonstrated encouraging efficacy with reduced proteinuria after 12 weeks of treatment (Wong, J Am Soc Nephrol 2020 31: 55A).

However, variability in complement activity and patient response was also observed, indicating highly potent compounds with greater and more sustained complement inhibition in vivo could provide greater therapeutic benefit to patients with C3G and a broad range of CKDs. Accordingly, it is desirable to provide compounds that inhibit complement factor B.

(S)-4-(2,2-difluoro-7-((5-methoxy-7-methyl-1H-indol-4-yl)methyl)-7-azaspiro[3.5]nonan-6-yl)benzoic acid, Formula I below, was disclosed in PCT/US2022/032042, filed on Jun. 3, 2022, and entitled “SUBSTITUTED INDOLE COMPOUNDS AND METHODS OF USE THEREOF” (incorporated by reference herein in its entirety).

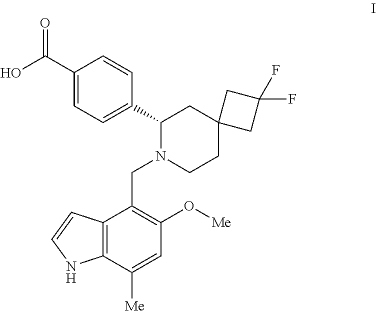 [SMILES: COc1cc(C)c2[nH]ccc2c1CN1CCC2(C[C@H]1c1ccc(C(=O)O)cc1)CC(F)(F)C2]

Solid state form of the active pharmaceutical ingredient (API) of a particular drug is often an important determinant of the drug's case of preparation, hygroscopicity, stability, solubility, storage stability, ease of formulation, rate of dissolution in gastrointestinal fluids and in vivo bioavailability. Salt formation is a technique for optimizing the aforementioned properties of ionizable drug candidates. Regardless of the presence of the counter ion, crystalline forms occur where the same composition of matter crystallizes in a different lattice arrangement resulting in different thermodynamic properties and stabilities specific to the particular crystalline form. Crystalline forms may also include different hydrates or solvates of the same compound. In deciding which form is preferable, the numerous properties of the forms are compared and the preferred form chosen based on the many physical property variables. It is entirely possible that one form can be preferable in some circumstances where certain aspects such as ease of preparation, stability, etc. are deemed to be critical. In other situations, a different form may be preferred for greater dissolution rate and/or superior bioavailability. It is not yet possible to predict whether a particular compound or salt of a compound will form polymorphs, whether any such polymorphs will be suitable for commercial use in a therapeutic composition, or which polymorphs will display such desirable properties.

### SUMMARY

Disclosed herein are crystalline forms of (S)-4-(2,2-difluoro-7-((5-methoxy-7-methyl-1H-indol-4-yl)methyl)-7-azaspiro[3.5]nonan-6-yl)benzoic acid.

In some embodiments, the crystalline form is Form A as described herein.

In some embodiments, Form A is prepared by a method comprising:
(a) adding (S)-4-(2,2-difluoro-7-((5-methoxy-7-methyl-1H-indol-4-yl)methyl)-7-azaspiro[3.5]nonan-6-yl)benzoic acid to isopropanol from about 40 to about 50° C. with stirring to form a solution of about 0.44 molar concentration;
(b) cooling the solution to about 20° C. and stirring for about 72 hours to form a suspension;
(c) filtering the suspension to obtain a solid;
(d) washing the solid with isopropanol; and
(e) drying the solid to provide the Form A.

Disclosed herein is a crystalline p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid.

In some embodiments, the crystalline form is Form B as described herein.

In some embodiments, Form B is prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding about 2.5 volumes of ethyl acetate;
(d) adding about 7.5 volumes of ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume;
(g) drying the solid to provide a solid form;
(h) adding the solid form to a binary mixture of isopropanol and water in an about 9:1 ratio by volume then heating at about 50° C. for about 16 hours to form a slurry;
(i) cooling the slurry to about 25° C. and filtering to obtain a solid; and
(j) drying the solid at about 50° C. to provide the Form B.

In some embodiments, the crystalline form is Form C as described herein.

In some embodiments, Form C is prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding about 2.5 volumes of ethyl acetate;
(d) adding about 7.5 volumes of ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume; and
(g) drying the solid to provide the Form C.

Disclosed herein is a crystalline hydrochloride salt form of (S)-4-(2,2-difluoro-7-((5-methoxy-7-methyl-1H-indol-4-yl)methyl)-7-azaspiro[3.5]nonan-6-yl)benzoic acid.

In some embodiments, the crystalline form is Form D as described herein.

In some embodiments, Form D is prepared by a method comprising:
(a) preparing a solution of (S)-4-(2,2-difluoro-7-((5-methoxy-7-methyl-1H-indol-4-yl)methyl)-7-azaspiro[3.5]nonan-6-yl)benzoic acid in about 5.5 volumes of ethyl acetate;
(b) adding about 0.55 equivalents of a 1 molar concentration solution of hydrogen chloride in ethyl acetate;
(c) adding a slurry of crystalline (S)-4-(2,2-difluoro-7-((5-methoxy-7-methyl-1H-indol-4-yl)methyl)-7-azaspiro[3.5]nonan-6-yl)benzoic acid hydrochloride in ethyl acetate to form a suspension;
(d) adding about 1.65 equivalents of a 1 molar concentration solution of hydrogen chloride in ethyl acetate to form a slurry;
(e) stirring the slurry at about 24° C. for about 16 hours;
(f) filtering the slurry to obtain a solid;
(g) washing the solid with ethyl acetate; and
(h) drying the solid to provide the Form D.

Some embodiments provide a pharmaceutical composition comprising one of Form A, Form B, Form C, or Form D, and a pharmaceutically acceptable carrier.

Some embodiments provide a pharmaceutical composition comprising Form A and a pharmaceutically acceptable carrier.

Some embodiments provide a pharmaceutical composition comprising Form B, Form C, or Form D, and a pharmaceutically acceptable carrier.

Disclosed herein is a method of treating a disease or disorder associated with complement factor B (CFB), comprising administering to a subject having such disease or disorder, a therapeutically effective amount of Form A, Form B, Form C, or Form D, or a pharmaceutical composition comprising Form A, Form B, Form C, or Form D, and a pharmaceutically acceptable carrier.

Disclosed herein is a method of treating a disease or disorder associated with complement factor B (CFB), comprising administering to a subject having such disease or disorder, a therapeutically effective amount of Form A, Form B, Form C, or Form D, or a pharmaceutical composition comprising Form B, Form C, or Form D, and a pharmaceutically acceptable carrier.

Some embodiments provide a method of treating or preventing a disease or disorder selected from the group consisting of: autoimmune disease or disorder, inflammatory disease or disorder, metabolic disease or disorder, neurological disease or disorder, pulmonary disease, respiratory disease or disorder, ophthalmic disease, cardiovascular disease, and kidney disease, comprising administering to a subject having such disease or disorder a therapeutically effective amount of Form A, Form B, Form C, or Form D, or a pharmaceutical composition comprising Form A, Form B, Form C, or Form D, and a pharmaceutically acceptable carrier.

Some embodiments provide a method of treating or preventing a disease or disorder selected from the group consisting of: autoimmune disease or disorder, inflammatory disease or disorder, metabolic disease or disorder, neurological disease or disorder, pulmonary disease, respiratory disease or disorder, ophthalmic disease, cardiovascular disease, and kidney disease, comprising administering to a subject having such disease or disorder a therapeutically effective amount of Form A, Form B, Form C, or Form D, or a pharmaceutical composition comprising Form B, Form C, or Form D, and a pharmaceutically acceptable carrier.

As used herein, when used to refer to modify a numerical value, the term “about” encompasses a range of uncertainty of the numerical value of from 0% to 10% of the numerical value.

The terms “polymorph” and “polymorphic form” refer to different crystalline forms of a single compound. That is, polymorphs are distinct solids sharing the same molecular formula, yet each polymorph may have distinct solid state physical properties. Therefore, a single compound may give rise to a variety of polymorphic forms where each form has different and distinct solid state physical properties, such as different solubility profiles, dissolution rates, melting point temperatures, flowability, and/or different X-ray diffraction peaks.

The term “amorphous” means a solid in a solid state that is a non-crystalline state. Amorphous solids are disordered arrangements of molecules and therefore possess no distinguishable crystal lattice or unit cell and consequently have no definable long range ordering. The solid state form of a solid may be determined by polarized light microscopy, X-ray powder diffraction (“XRPD”), differential scanning calorimetry (“DSC”), or other standard techniques known to those of skill in the art.

As used herein, a compound is “substantially pure” if the compound contains an insignificant amount of other components. Such components can include, for example, starting materials, residual solvents, other polymorphic or crystalline forms, the opposite enantiomer, other salt forms, other solvates, or any other impurities that can result from the preparation, isolation, and/or recrystallization of the compounds provided herein. In some embodiments, the other components can include, for example, starting materials, residual solvents, other polymorphic or crystalline forms, the opposite enantiomer, other salt forms, or any other impurities that can result from the preparation, isolation, and/or recrystallization of the compounds provided herein. In some embodiments, a solid form (e.g., a particular crystalline form or salt) of (S)-4-(2,2-difluoro-7-((5-methoxy-7-methyl-1H-indol-4-yl)methyl)-7-azaspiro[3.5]nonan-6-yl)benzoic acid is “substantially pure” if the solid form consists of at least about 95% by weight of the solid form. In some embodiments, a solid form of (S)-4-(2,2-difluoro-7-((5-methoxy-7-methyl-1H-indol-4-yl)methyl)-7-azaspiro[3.5]nonan-6-yl)benzoic acid is “substantially pure” if the solid form constitutes at least about 97%, about 98%, about 99%, or about 99.5% by weight of the solid form.

The terms “effective amount” and “therapeutically effective amount” are used interchangeably herein, refers to an amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In particular, an effective amount, when administered to a subject in need of such treatment, is sufficient to (i) treat or prevent a particular disease, condition, or disorder which can be treated with an inhibitor of CFB, (ii) attenuate, ameliorate, or eliminate one or more symptoms of the particular disease, condition, or disorder, or (iii) prevent or delay the onset of one or more symptoms of the particular disease, condition, or disorder described herein. The amount of the crystalline forms described herein that will correspond to such a therapeutically effective amount will vary depending upon factors such the disease condition and its severity, the identity (e.g., weight) of the mammal in need of treatment.

The term “free form” refers to a compound in a non-salt form.

The term “hydrate” means a compound or salt thereof, further including a stoichiometric or non-stoichiometric amount of water bound by non-covalent intermolecular forces. The term “anhydrate” means a compound or salt thereof, not including a stoichiometric or non-stoichiometric amount of water bound by non-covalent intermolecular forces.

The term “pharmaceutical composition” as used herein is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier.

The term “pharmaceutically acceptable carrier” refers to a carrier or an adjuvant that may be administered to a patient, together with a compound of the present disclosure, or a pharmaceutically acceptable salt thereof, and which does not destroy the pharmacological activity thereof and is nontoxic when administered in doses sufficient to deliver a therapeutic amount of the compound.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The details of one or more embodiments of the disclosure are set forth in the accompanying drawings and description below. Other features and advantages of the disclosure will be apparent from the description, the drawings, and from the claims.

### DESCRIPTION OF DRAWINGS

FIG. 1 is an X-ray powder diffractogram of amorphous (S)-4-(2,2-difluoro-7-((5-methoxy-7-methyl-1H-indol-4-yl)methyl)-7-azaspiro[3.5]nonan-6-yl)benzoic acid.

FIG. 2 is an X-ray powder diffractogram of Form A.

FIG. 3 is a thermogravimetric analysis thermogram of Form A.

FIG. 4 is a differential scanning calorimetry pattern of Form A.

FIG. 5 is an X-ray powder diffractogram of Form A (alternative preparation).

FIG. 6 is a differential scanning calorimetry pattern of Form A (alternative preparation).

FIG. 7 is a thermogravimetric analysis thermogram of Form A (alternative preparation).

FIG. 8 is a ¹H NMR spectrum of Form A (alternative preparation).

FIG. 9 is a scanning electron microscope image of Form A (alternative preparation).

FIG. 10 is an intrinsic dissolution rate curve of Form A (alternative preparation) in pH 2.0 HCl buffer.

FIG. 11 is an intrinsic dissolution rate curve of Form A (alternative preparation) in pH 6.5 phosphate buffer.

FIG. 12 is an X-ray powder diffractogram of Form B.

FIG. 13 is a thermogravimetric analysis thermogram of Form B.

FIG. 14 shows the differential scanning calorimetry pattern of Form B.

FIG. 15 is an X-ray powder diffractogram of Form B (alternative preparation).

FIG. 16 is a differential scanning calorimetry pattern of Form B (alternative preparation).

FIG. 17 is a thermogravimetric analysis thermogram of Form B (alternative preparation).

FIG. 18 is a ¹H NMR spectrum of Form B (alternative preparation).

FIG. 19 is a scanning electron microscope image of Form B (alternative preparation).

FIG. 20 is an intrinsic dissolution rate of Form B in pH 2 buffer.

FIG. 21 is an intrinsic dissolution rate of Form B in pH 6.5 buffer.

FIG. 22 is a dissolution profile of a capsule containing Form B.

FIG. 23 is an X-ray powder diffractogram of Form C.

FIG. 24 is a thermogravimetric analysis thermogram of Form C.

FIG. 25 is a differential scanning calorimetry pattern of Form C.

FIG. 26 is an X-ray powder diffractogram of Form D.

FIG. 27 is a thermogravimetric analysis thermogram of Form D.

FIG. 28 is a differential scanning calorimetry pattern of Form D.

### DETAILED DESCRIPTION

Featured herein are crystalline forms of (S)-4-(2,2-difluoro-7-((5-methoxy-7-methyl-1H-indol-4-yl)methyl)-7-azaspiro[3.5]nonan-6-yl)benzoic acid (Formula I) below) and salts thereof.

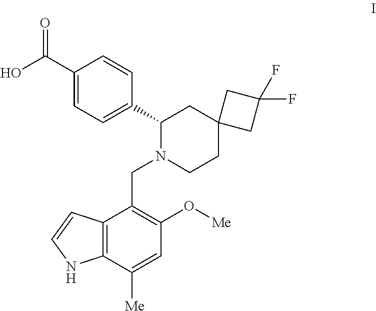 [SMILES: COc1cc(C)c2[nH]ccc2c1CN1CCC2(C[C@H]1c1ccc(C(=O)O)cc1)CC(F)(F)C2]

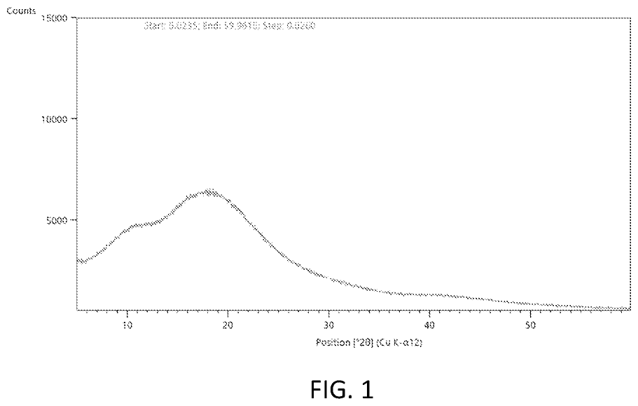

Also provided herein is amorphous (S)-4-(2,2-difluoro-7-((5-methoxy-7-methyl-1H-indol-4-yl)methyl)-7-azaspiro[3.5]nonan-6-yl)benzoic acid. In some embodiments, the amorphous (S)-4-(2,2-difluoro-7-((5-methoxy-7-methyl-1H-indol-4-yl)methyl)-7-azaspiro[3.5]nonan-6-yl)benzoic acid is characterized by an XRPD pattern that is substantially the same as that shown in FIG. 1.

### Form A

The present disclosure provides a crystalline form of the free form of (S)-4-(2,2-difluoro-7-((5-methoxy-7-methyl-1H-indol-4-yl)methyl)-7-azaspiro[3.5]nonan-6-yl)benzoic acid (the compound of Formula I). In one embodiment, the free form is an anhydrate form. The crystalline form of the free form of the compound of Formula I that is the anhydrate form is herein referred to as Form A. Form A is described and characterized herein.

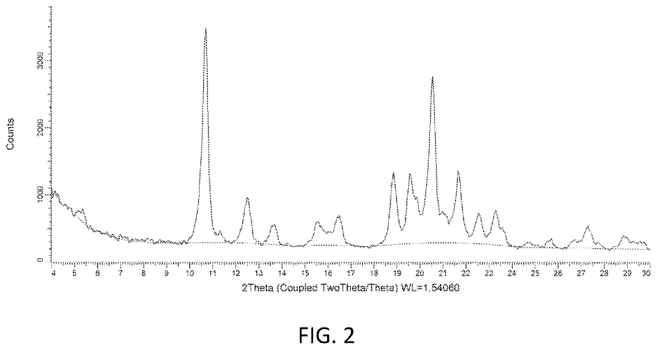

In some embodiments, the crystalline form is Form A, and the XRPD pattern is substantially the same as that shown in FIG. 2.

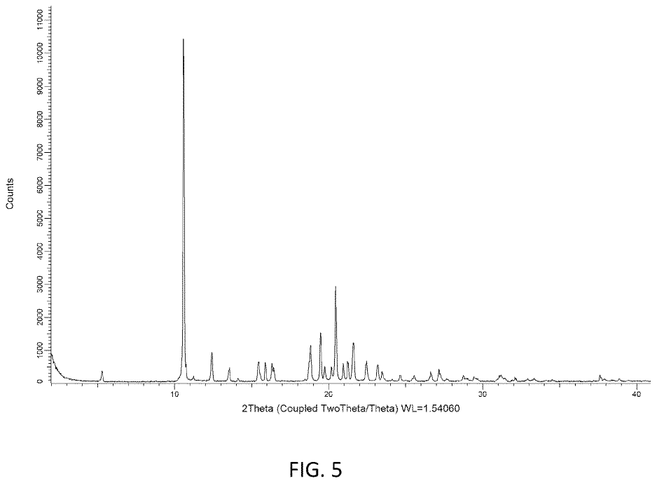

In some embodiments, the crystalline form is Form A, and the XRPD pattern is substantially the same as that shown in FIG. 5.

In some embodiments, the crystalline form is Form A, and the XRPD pattern is represented by peaks shown in the table below:

| Angle | d Value | Rel. Intensity | Rel. Intensity Rank |
|---|---|---|---|
| 5.296° | 16.67276 Å | 3.0% | 15 |
| 10.586° | 8.35026 Å | 100.0% | 1 |
| 12.424° | 7.11890 Å | 8.4% | 6 |
| 13.559° | 6.52547 Å | 3.9% | 14 |
| 15.459° | 5.72733 Å | 5.7% | 8 |
| 15.900° | 5.56937 Å | 5.1% | 10 |
| 16.314° | 5.42897 Å | 5.1% | 11 |
| 18.822° | 4.71095 Å | 10.4% | 5 |
| 19.477° | 4.55390 Å | 14.1% | 3 |
| 20.459° | 4.33740 Å | 27.6% | 2 |
| 20.953° | 4.23639 Å | 4.9% | 13 |
| 21.243° | 4.17919 Å | 5.2% | 9 |
| 21.601° | 4.11072 Å | 11.0% | 4 |
| 22.458° | 3.95579 Å | 5.8% | 7 |
| 23.173° | 3.83519 Å | 5.0% | 12 |

In some embodiments, the crystalline form is characterized by an X-ray powder diffraction (XRPD) pattern having a peak at 10.7±0.2 degrees 2θ. The X-ray powder diffraction pattern of Form A can also include one or more additional characteristic peaks.

In some embodiments, the X-ray powder diffraction pattern of Form A can also include one or more of the following additional characteristic peaks, which can also be used to identify Form A (e.g., in a sample).

For example, the XRPD pattern has a peak at 20.5±0.2 degrees 2θ.

For example, the XRPD pattern has a peak at 18.8±0.2 degrees 2θ.

For example, the XRPD pattern has a peak at 21.7±0.2 degrees 2θ.

For example, the XRPD pattern has a peak at 19.6±0.2 degrees 2θ.

For example, the XRPD pattern has a peak at 19.8±0.2 degrees 2θ.

For example, the XRPD pattern has a peak at 12.5±0.2 degrees 2θ.

For example, the XRPD pattern has a peak at 21.7±0.2 degrees 2θ.

For example, the XRPD pattern has a peak at 23.3±0.2 degrees 2θ.

For example, the XRPD pattern has a peak at 22.5±0.2 degrees 2θ.

For example, the XRPD pattern has a peak at 27.3±0.2 degrees 2θ.

For example, the XRPD pattern has a peak at 15.5±0.2 degrees 2θ.

The X-ray powder diffraction pattern of Form A can also include one or more lower intensity characteristic peaks. The relative intensities of these additional peak(s) are, in general, lower than the relative intensities associated with the characteristic peaks described above.

For example, the XRPD pattern has a peak at 5.3±0.2 degrees 2θ.

For example, the XRPD pattern has a peak at 13.7±0.2 degrees 2θ.

For example, the XRPD pattern has a peak at 16.5±0.2 degrees 2θ.

For example, the XRPD pattern has a peak at 15.6±0.2 degrees 2θ.

For example, the XRPD pattern has a peak at 23.5±0.2 degrees 2θ.

For example, the XRPD pattern has a peak at 16.1±0.2 degrees 2θ.

For example, the XRPD pattern has a peak at 28.9±0.2 degrees 2θ.

For example, the XRPD pattern has a peak at 11.3±0.2 degrees 2θ.

For example, the XRPD pattern has a peak at 26.7±0.2 degrees 2θ.

For example, the XRPD pattern has a peak at 25.6±0.2 degrees 2θ.

For example, the XRPD pattern has a peak at 29.2±0.2 degrees 2θ.

In some embodiments, the crystalline form is Form A, and the XRPD pattern has peaks (±0.2 degrees 2θ) at 10.7, 20.5, and 18.8.

In some embodiments, the crystalline form is Form A, and the XRPD pattern has peaks (±0.2 degrees 2θ) at 10.7, 20.5, 18.8, and 21.7.

In some embodiments, the crystalline form is Form A, and the XRPD pattern has peaks (±0.2 degrees 2θ) at 10.7, 20.5, 18.8, 21.7, 19.6, 19.8, 12.5, 21.1, 23.3, 22.5, 27.3, and 15.55.

In some embodiments, the crystalline form is Form A, and the XRPD pattern has peaks (±0.2 degrees 2θ) at 10.7, 20.5, 18.8, 21.7, 19.6 19.8, 12.5, 21.1, 23.3, 22.5, 27.3, 15.5, 5.3, 13.7, 16.5, 15.6, 23.5, 16.1, 28.7, 11.3, 26.7, 25.6, and 29.2.

In some embodiments, the crystalline form is Form A, and the XRPD pattern has a peak (±0.2 degrees 2θ) at 10.7.

In some embodiments, the crystalline form is Form A, and the XRPD pattern has peaks (±0.2 degrees 2θ) at 10.7 and 20.5.

In some embodiments, the crystalline form is Form A, and the XRPD pattern has peaks (±0.2 degrees 2θ) at 10.7, 20.5, and 19.5.

In some embodiments, the crystalline form is Form A, and the XRPD pattern has peaks (±0.2 degrees 2θ) at 10.7, 20.5, 19.5, and 21.6.

In some embodiments, the crystalline form is Form A, and the XRPD pattern has peaks (±0.2 degrees 2θ) at 10.7, 20.5, 19.5, 21.6, and 18.8.

In some embodiments, the crystalline form is Form A, and the XRPD pattern has peaks (±0.2 degrees 2θ) at 10.7, 20.5, 19.5, 21.6, 18.8, and 12.4.

In some embodiments, the crystalline form is Form A, and the XRPD pattern has peaks (±0.2 degrees 2θ) at 10.7, 20.5, 19.5, 21.6, 18.8, 12.4, and 22.5.

In some embodiments, the crystalline form is Form A, and the XRPD pattern has peaks (±0.2 degrees 2θ) at 10.7, 20.5, 19.5, 21.6, 18.8, 12.4, 22.5, and 15.5.

In some embodiments, the crystalline form is Form A, and the XRPD pattern has peaks (±0.2 degrees 2θ) at 10.7, 20.5, 19.5, 21.6, 18.8, 12.4, 22.5, 15.5, and 21.2.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, and 15.9.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, 15.9, and 16.3.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, 15.9, 16.3, and 23.2.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, 15.9, 16.3, 23.2, and 20.9.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, 15.9, 16.3, 23.2, 20.9, and 13.6.

In some embodiments, the crystalline form is Form A, and the XRPD pattern has peaks (±0.2 degrees 2θ) at 10.7, 20.5, 19.5, 21.6, 18.8, 12.4, 22.5, 15.5, 21.2, 15.9, 16.3, 23.2, 20.9, 13.6, and 5.3.

The skilled artisan will recognize that the relative intensities of X-ray powder diffraction pattern peaks can vary depending on the sample preparation technique, crystal size distribution, various filters used, sample mounting procedure, and the particular instrument employed. Accordingly, depending on the type of the instrument and settings employed (including filters), new peaks may be observed in subsequently obtained patterns; or peaks observed in previously obtained patterns may have a negligible relative intensities (and hence may not be observed) in subsequently obtained patterns. As such, the absence of one or more of the lower relative intensity peaks described above may not, in and of itself, establish that Form A is not present (e.g., in a sample). However, the presence of the lower relative intensity peaks described above can, in general, be used to further establish the presence of Form A in a sample.

Form A can also have one or more of the following characteristics.

In some embodiments, the crystalline form is Form A having a thermogravimetric analysis (TGA) curve characterized by a weight loss of about 0.5% to about 4% (e.g., about 1% to about 3%, or about 2%) at about 150° C. to about 220° C. (e.g., about 150° C. to about 190° C., about 165° C. to about 205° C., about 170° C. to about 220° C., about 180° C. to about 200° C., about 185° C. to about 195° C., about 187° C. to about 191° C., or about 189° C. In some embodiments, the crystalline form is Form A having a thermogravimetric analysis (TGA) curve characterized by a weight loss of about 2% at about 189° C.

In some embodiments, the crystalline form is Form A having a TGA curve characterized by a weight loss of about 15% to about 35% (e.g., about 20% to about 27%, about 23% to about 35%, about 20% to about 30%, about 23% to about 25% at about 270 to about 330 (e.g., about 280° C. to about 320° C., about 290° C. to about 310° C., about 295° C. to about 305° C., or about 300° C.). In some embodiments, the crystalline form is Form A having a TGA curve characterized by a weight loss of about 25% at about 300° C.

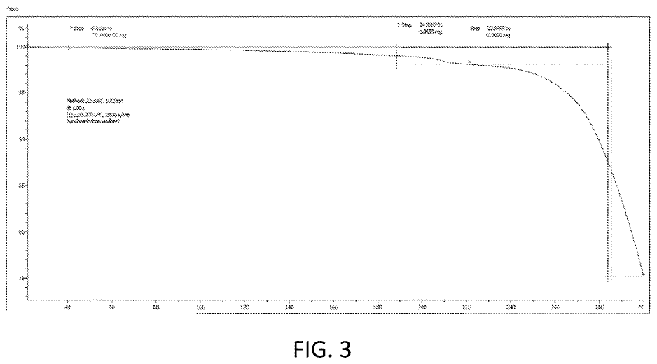

In some embodiments, the crystalline form is Form A having a TGA curve that is substantially the same as that shown in FIG. 3.

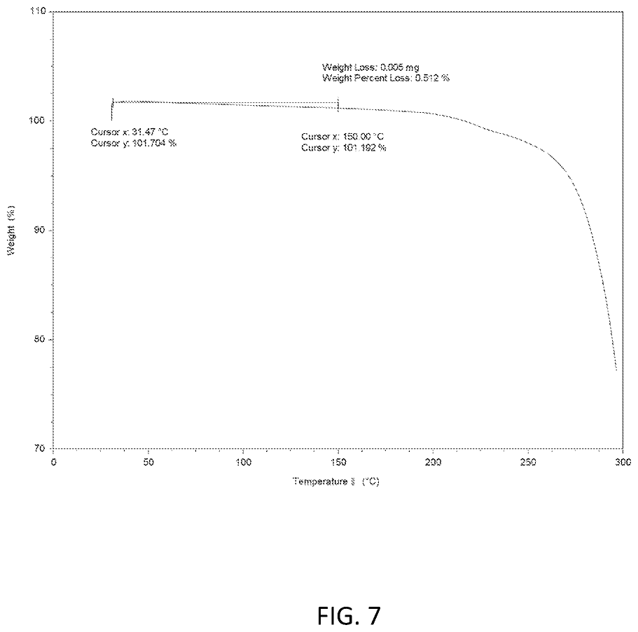

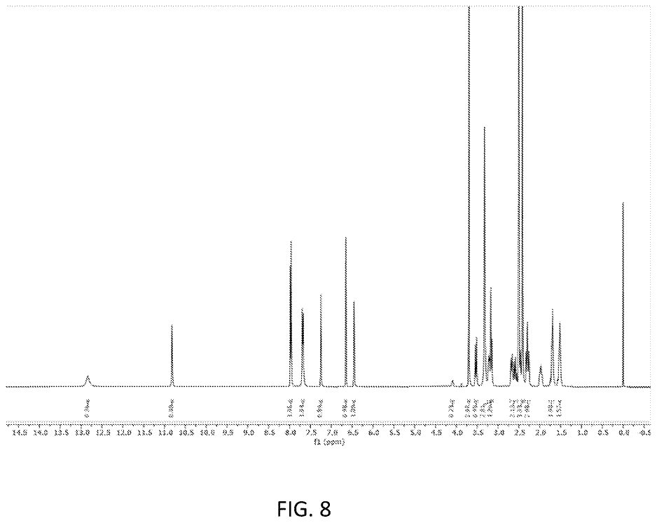

In some embodiments, the crystalline form is Form A having a TGA curve that is substantially the same as that shown in FIG. 7.

In some embodiments, the crystalline form is Form A having a differential scanning calorimetry (DSC) curve characterized by a melting onset of about 185° C. to about 220° C., about 190° C. to about 215° C., about 195° C. to about 208° C., about 198° C. to about 207° C., about 200° C. to about 204° C., about 201° C. to about 203° C., or about 201.9° C. (endo). In some embodiments, the crystalline form is Form A having a differential scanning calorimetry (DSC) curve characterized by a melting onset of about 201.9° C. (endo).

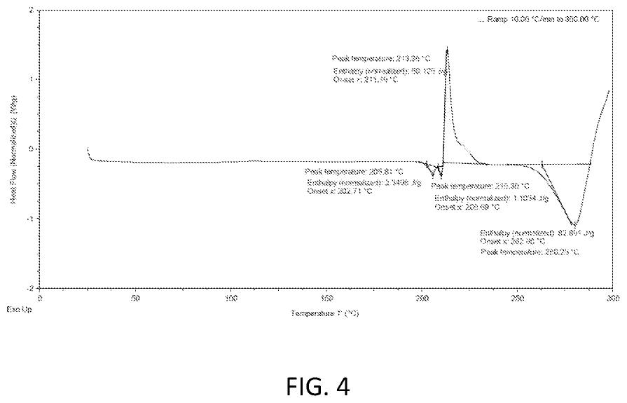

In some embodiments, the crystalline form is Form A having a DSC curve that is substantially the same as that shown in FIG. 4.

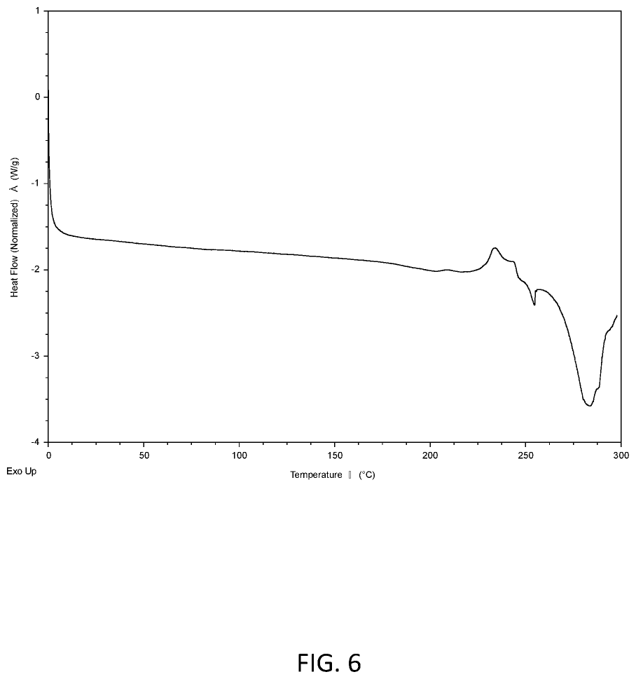

In some embodiments, the crystalline form is Form A having a DSC curve that is substantially the same as that shown in FIG. 6.

In some embodiments, Form A has a TGA diagram when heated from 30° C. and 300° C. at 10 K/min showing a weight loss of about 0.51% at 150° C.

In some embodiments, Form A absorbs up to 0.45% of moisture at 95% RH at 25° C. by DVS.

In some embodiments, the crystalline form is Form A characterized by having a solubility of about 1.8 mg/mL to about 2.8 mg/mL in Fasted State Simulated Gastric Fluid (FaSSGF) at about 37° C. after about 30 minutes, for example, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, or any value in between. In some embodiments, Form A is characterized by having a solubility of about 2.2 mg/mL to about 2.5 mg/mL in Fasted State Simulated Gastric Fluid (FaSSGF) at about 37° C. after about 30 minutes. In some embodiments, Form A is characterized by having a solubility of 2.2 mg/mL to 2.5 mg/mL in Fasted State Simulated Gastric Fluid (FaSSGF) at about 37° C. after about 30 minutes. In some embodiments, Form A is characterized by having a solubility of about 2.3 mg/mL in Fasted State Simulated Gastric Fluid (FaSSGF) at about 37° C. after about 30 minutes.

In some embodiments, the crystalline form is Form A characterized by having a solubility of greater than about 1.7 mg/mL to greater than about 2 mg/mL in Fasted State Simulated Gastric Fluid (FaSSGF) at about 37° C. after about 24 hours, for example, greater than about 1.7 mg/mL, greater than about 1.8 mg/mL, greater than about 1.9 mg/mL, or greater than about 2.0 mg/mL, such as about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3.0 mg/mL. In some embodiments, Form A is characterized by having a solubility greater than about 1.8 mg/mL to greater than about 1.9 mg/mL in Fasted State Simulated Gastric Fluid (FaSSGF) at about 37° C. after about 24 hours. In some embodiments, Form A is characterized by having a solubility of greater than about 1.9 mg/mL to greater than about 2 mg/mL in Fasted State Simulated Gastric Fluid (FaSSGF) at about 37° C. after about 24 hours. In some embodiments, Form A is characterized by having a solubility of greater than about 2 mg/mL in Fasted State Simulated Gastric Fluid (FaSSGF) at about 37° C. after about 24 hours.

In some embodiments, the crystalline form is Form A characterized by having a solubility of greater than 2 mg/mL in Fasted State Simulated Gastric Fluid (FaSSGF) at an initial pH of 1.6 at about 25° C. after about 24 hours.

In some embodiments, the crystalline form is Form A characterized by having a solubility of about 0.26 mg/mL to about 0.36 mg/mL in Fed State Simulated Intestinal Fluid (FeSSIF) at about 37° C. after about 30 minutes, for example, about 0.26 mg/mL, about 0.27 mg/mL, about 0.28 mg/mL, about 0.29 mg/mL, about 0.30 mg/mL, about 0.31 mg/mL, about 0.32 mg/mL, about 0.33 mg/mL, about 0.34 mg/mL, about 0.35 mg/mL, about 0.36 mg/mL, or any value in between. In some embodiments, Form A is characterized by having a solubility of about 0.29 mg/mL to about 0.33 mg/mL in Fed State Simulated Intestinal Fluid (FeSSIF) at about 37° C. after about 30 minutes. In some embodiments, Form A is characterized by having a solubility of 0.30 mg/mL to 0.32 mg/mL in Fed State Simulated Intestinal Fluid (FeSSIF) at about 37° C. after about 30 minutes. In some embodiments, Form A is characterized by having a solubility of about 0.31 mg/mL in Fed State Simulated Intestinal Fluid (FeSSIF) at about 37° C. after about 30 minutes.

In some embodiments, the crystalline form is Form A characterized by having a solubility of about 0.26 mg/mL to about 0.36 mg/mL in Fed State Simulated Intestinal Fluid (FeSSIF) at about 37° C. after about 24 hours, for example, about 0.26 mg/mL, about 0.27 mg/mL, about 0.28 mg/mL, about 0.29 mg/mL, about 0.30 mg/mL, about 0.31 mg/mL, about 0.32 mg/mL, about 0.33 mg/mL, about 0.34 mg/mL, about 0.35 mg/mL, about 0.36 mg/mL, or any value in between. In some embodiments, Form A is characterized by having a solubility of about 0.30 mg/mL to about 0.32 mg/mL in Fed State Simulated Intestinal Fluid (FeSSIF) at about 37° C. after about 24 hours. In some embodiments, Form A is characterized by having a solubility of 0.30 mg/mL to 0.32 mg/mL in Fed State Simulated Intestinal Fluid (FeSSIF) at about 37° C. after about 24 hours. In some embodiments, Form A is characterized by having a solubility of about 0.31 mg/mL in Fed State Simulated Intestinal Fluid (FeSSIF) at about 37° C. after about 24 hours.

In some embodiments, the crystalline form is Form A characterized by having a solubility of about 0.18 mg/mL to about 0.28 mg/mL in Fasted State Simulated Intestinal Fluid (FaSSIF) at about 37° C. after about 30 minutes, for example, about 0.18 mg/mL, about 0.19 mg/mL, about 0.20 mg/mL, about 0.21 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, or any value in between. In some embodiments, Form A is characterized by having a solubility of about 0.20 mg/mL to about 0.26 mg/mL in Fasted State Simulated Intestinal Fluid (FaSSIF) at about 37° C. after about 30 minutes. In some embodiments, Form A is characterized by having a solubility of 0.23 mg/mL to 0.25 mg/mL in Fasted State Simulated Intestinal Fluid (FaSSIF) at about 37° C. after about 30 minutes. In some embodiments, the crystalline form is Form A characterized by having a solubility of about 0.24 mg/mL in Fasted State Simulated Intestinal Fluid (FaSSIF) at about 37° C. after about 30 minutes.

In some embodiments, the crystalline form is Form A characterized by having a solubility of about 0.13 mg/mL to about 0.23 mg/mL in Fasted State Simulated Intestinal Fluid (FaSSIF) at about 37° C. after about 24 hours, for example, about 0.13 mg/mL, about 0.14 mg/mL, about 0.15 mg/mL, about 0.16 mg/mL, about 0.17 mg/mL, about 0.18 mg/mL, about 0.19 mg/mL, about 0.20 mg/mL, about 0.21 mg/mL, about 0.22 mg/mL, about 0.23 mg/mL, or any value in between. In some embodiments, Form A is characterized by having a solubility of about 0.15 mg/mL to about 0.20 mg/mL in Fasted State Simulated Intestinal Fluid (FaSSIF) at about 37° C. after about 24 hours. In some embodiments, Form A is characterized by having a solubility of 0.17 mg/mL to 0.19 mg/mL in Fasted State Simulated Intestinal Fluid (FaSSIF) at about 37° C. after about 24 hours. In some embodiments, the crystalline form is Form A characterized by having a solubility of about 0.18 mg/mL in Fasted State Simulated Intestinal Fluid (FaSSIF) at about 37° C. after about 24 hours.

In some embodiments, the crystalline form is Form A characterized by having a solubility of about 0.07 mg/mL in Fasted State Simulated Intestinal Fluid (FaSSIF) at an initial pH of 6.5 at about 25° C. after about 24 hours. In some embodiments, the Fasted State Simulated Intestinal Fluid (FaSSIF) comprises a phosphate buffer saline (PBS).

In some embodiments, the crystalline form is Form A characterized by having a solubility of about 0.07 mg/mL in a phosphate buffer saline (PBS) at an initial pH of 6.5 at about 25° C. after about 24 hours. In some embodiments, a phosphate buffer saline (PBS) comprises sodium taurocholate (NaTC). In some embodiments, a phosphate buffer saline (PBS) comprises sodium taurocholate (NaTC) at 3 mM.

In some embodiments, the crystalline form is Form A characterized by having a solubility of about 0.16 mg/mL to about 0.26 mg/mL in water with an initial pH of about 7.0 at about 37° C. after about 30 minutes, for example, about 0.16 mg/mL, about 0.17 mg/mL, about 0.18 mg/mL, about 0.19 mg/mL, about 0.20 mg/mL, about 0.21 mg/mL, about 0.22 mg/mL, about 0.23 mg/mL, about 0.24 mg/mL, about 0.25 mg/mL, about 0.26 mg/mL, or any value in between. In some embodiments, Form A is characterized by having a solubility of about 0.17 mg/mL to about 0.23 mg/mL in water with an initial pH of about 7.0 at about 37° C. after about 30 minutes. In some embodiments, Form A is characterized by having a solubility of 0.20 mg/mL to 0.22 mg/mL in water with an initial pH of about 7.0 at about 37° C. after about 30 minutes. In some embodiments, the crystalline form is Form A characterized by having a solubility of about 0.21 mg/mL in water with an initial pH of about 7.0 at about 37° C. after about 30 minutes.

In some embodiments, the crystalline form is Form A characterized by having a solubility of about 0.17 mg/mL to about 0.27 mg/mL in water with an initial pH of about 7.0 at about 37° C. after about 24 hours, for example, about 0.17 mg/mL, about 0.18 mg/mL, about 0.19 mg/mL, about 0.20 mg/mL, about 0.21 mg/mL, about 0.22 mg/mL, about 0.23 mg/mL, about 0.24 mg/mL, about 0.25 mg/mL, about 0.26 mg/mL, about 0.27 mg/mL, or any value in between. In some embodiments, Form A is characterized by having a solubility of about 0.20 mg/mL to about 0.24 mg/mL in water with an initial pH of about 7.0 at about 37° C. after about 30 minutes. In some embodiments, Form A is characterized by having a solubility of 0.20 mg/mL to 0.23 mg/mL in water with an initial pH of about 7.0 at about 37° C. after about 30 minutes. In some embodiments, the crystalline form is Form A characterized by having a solubility of about 0.22 mg/mL in water with an initial pH of about 7.0 at about 37° C. after about 24 hours.

In some embodiments, the crystalline form is Form A characterized by having a solubility of about 0.05 mg/mL in water with an initial pH of about 7.0 at about 25° C. after 24 hrs.

In some embodiments, the crystalline form is Form A, in substantially pure form. In some embodiments, the crystalline form is Form A, wherein the Form A is at least 99%, 99.3%, 99.5%, 99.7%, or 99.9% pure (w/w). In some embodiments, the crystalline form is Form A, wherein the Form A is at least 99%, 99.3%, 99.5%, 99.7%, or 99.9% pure (w/w) after 1 week of exposure to about 10% relative humidity at about 50° C. In some embodiments, the crystalline form is Form A, wherein the Form A is at least 99%, 99.3%, 99.5%, 99.7%, or 99.9% pure (w/w) after 1 week of exposure to about 10% relative humidity at about 80° C. In some embodiments, the crystalline form is Form A, wherein the Form A is at least 99%, 99.3%, 99.5%, 99.7%, or 99.9% pure (w/w) after 1 week of exposure to about 75% relative humidity at about 50° C. In some embodiments, the crystalline form is Form A, wherein the Form A is at least 99%, 99.3%, 99.5%, 99.7%, or 99.9% pure (w/w) after 1 week of exposure to about 75% relative humidity at about 80° C.

### Form B

The present disclosure provides (S)-4-(2,2-difluoro-7-((5-methoxy-7-methyl-1H-indol-4-yl)methyl)-7-azaspiro[3.5]nonan-6-yl)benzoic acid p-toluene sulfonate (Formula II). The terms “(S)-4-(2,2-difluoro-7-((5-methoxy-7-methyl-1H-indol-4-yl)methyl)-7-azaspiro[3.5]nonan-6-yl)benzoic acid p-toluene sulfonate” and “(S)-4-(2,2-difluoro-7-((5-methoxy-7-methyl-1H-indol-4-yl)methyl)-7-azaspiro[3.5]nonan-6-yl)benzoic acid p-toluene sulfonic acid salt” are used herein interchangeably.

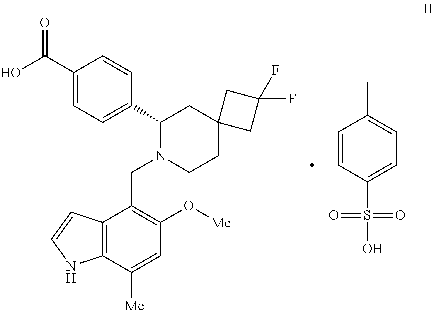 [SMILES: COc1cc(C)c2[nH]ccc2c1CN1CCC2(C[C@H]1c1ccc(C(=O)O)cc1)CC(F)(F)C2 ; Cc1ccc(S(=O)(=O)O)cc1]

The present disclosure provides a crystalline form of (S)-4-(2,2-difluoro-7-((5-methoxy-7-methyl-1H-indol-4-yl)methyl)-7-azaspiro[3.5]nonan-6-yl)benzoic acid p-toluene sulfonate (herein referred to as Form B). In some embodiments, Form B includes water, wherein water is in an amount from 0% to 2.7% w/w relative to a total weight of Form B. The water content may vary according to the drying condition and ambient humidity. In some embodiment, water is in an amount from 1.5% to 2.5% w/w under ambient conditions. In some embodiment, water is in an amount from 1.5% to 2% w/w under ambient conditions.

Form B is a high crystalline form with a thick plate like morphology. Form B is likely a nonstoichiometric hydrate form and is characterized as a channel hydrate and shows a reversible water sorption and desorption behavior with no hysteresis. Form B shows no change in the XRPD pattern across a wide range of conditions including across the range of ambient humidity and temperature.

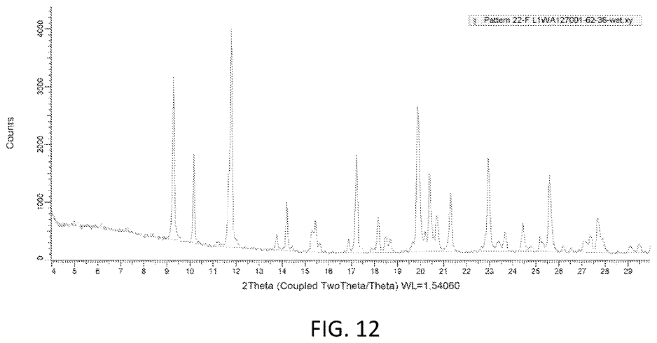

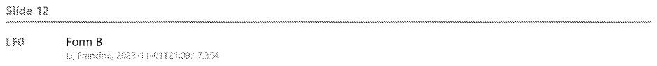

In some embodiments, the crystalline form is Form B, and the XRPD pattern is substantially the same as that shown in FIG. 12.

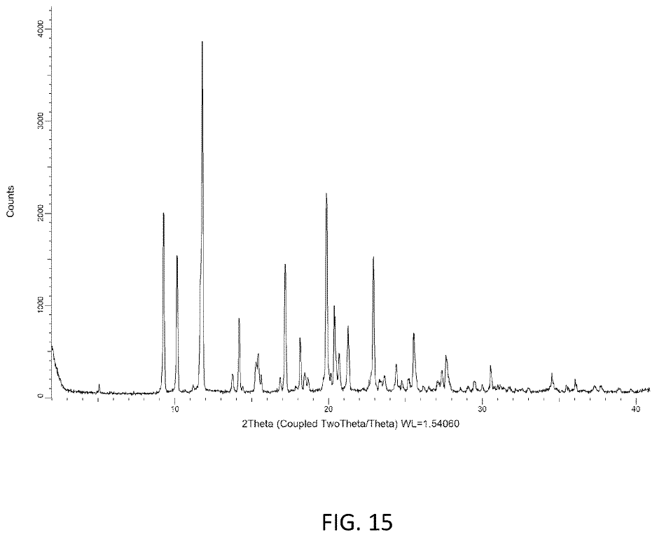

In some embodiments, the crystalline form is Form B, and the XRPD pattern is substantially the same as that shown in FIG. 15.

In some embodiments, the crystalline form is Form B, and the XRPD pattern is represented by peaks shown in the table below:

| Angle | d Value | | Rel. Intensity | Rel. Intensity Rank |
|---|---|---|---|---|
| 9.280° | 9.52241 | Å | 51.6% | 3 |
| 10.148° | 8.70982 | Å | 39.1% | 4 |
| 18.151° | 4.88347 | Å | 15.2% | 10 |
| 11.790° | 7.50015 | Å | 100.0% | 1 |
| 14.185° | 6.23850 | Å | 21.1% | 8 |
| 15.428° | 5.73861 | Å | 10.8% | 12 |
| 17.187° | 5.15508 | Å | 36.2% | 6 |
| 19.864° | 4.46597 | Å | 55.9% | 2 |
| 20.364° | 4.35744 | Å | 23.8% | 7 |
| 20.696° | 4.28836 | Å | 10.1% | 13 |
| 21.278° | 4.17232 | Å | 18.4% | 9 |
| 22.915° | 3.87778 | Å | 38.4% | 5 |
| 24.396° | 3.64563 | Å | 7.4% | 15 |
| 25.549° | 3.48375 | Å | 15.2% | 11 |
| 27.633° | 3.22558 | Å | 10.1% | 14 |

In some embodiments, the crystalline form is characterized by an XRPD pattern having a peak at 11.8±0.2 degrees 2θ. The X-ray powder diffraction pattern of Form B can also include one or more additional characteristic peaks.

In some embodiments, the X-ray powder diffraction pattern of Form B can also include one or more of the following additional characteristic peaks, which can also be used to identify Form B (e.g., in a sample).

For example, the XRPD pattern has a peak at 9.3±0.2 degrees 2θ.

For example, the XRPD pattern has a peak at 19.9±0.2 degrees 2θ.

For example, the XRPD pattern has a peak at 22.9±0.2 degrees 2θ.

For example, the XRPD pattern has a peak at 17.2±0.2 degrees 2θ.

For example, the XRPD pattern has a peak at 10.2±0.2 degrees 2θ.

For example, the XRPD pattern has a peak at 20.4±0.2 degrees 2θ.

For example, the XRPD pattern has a peak at 21.3±0.2 degrees 2θ.

For example, the XRPD pattern has a peak at 14.2±0.2 degrees 2θ.

The X-ray powder diffraction pattern of Form B can also include one or more lower intensity characteristic peaks. The relative intensities of these additional peak(s) are, in general, lower than the relative intensities associated with the characteristic peaks described above.

For example, the XRPD pattern has a peak at 18.2±0.2 degrees 2θ.

For example, the XRPD pattern has a peak at 20.7±0.2 degrees 2θ.

For example, the XRPD pattern has a peak at 15.4±0.2 degrees 2θ.

For example, the XRPD pattern has a peak at 24.4±0.2 degrees 2θ.

For example, the XRPD pattern has a peak at 20.2±0.2 degrees 2θ.

For example, the XRPD pattern has a peak at 23.7±0.2 degrees 2θ.

For example, the XRPD pattern has a peak at 15.3±0.2 degrees 2θ.

For example, the XRPD pattern has a peak at 25.2±0.2 degrees 2θ.

For example, the XRPD pattern has a peak at 18.7±0.2 degrees 2θ.

For example, the XRPD pattern has a peak at 18.5±0.2 degrees 2θ.

For example, the XRPD pattern has a peak at 16.9±0.2 degrees 2θ.

For example, the XRPD pattern has a peak at 13.8±0.2 degrees 2θ.

In some embodiments, the crystalline form is Form B, and the XRPD pattern has peaks (±0.2 degrees 2θ) at 11.8, 9.3, and 19.9.

In some embodiments, the crystalline form is Form B, and the XRPD pattern has peaks (±0.2 degrees 2θ) at 11.8, 9.3, 19.9, and 22.9.

In some embodiments, the crystalline form is Form B, and the XRPD pattern has peaks (±0.2 degrees 2θ) at 11.8, 9.3, 19.9, 22.9, 17.2, 10.2, 20.4, 21.3, and 14.2.

In some embodiments, the crystalline form is Form B, and the XRPD pattern has peaks (±0.2 degrees 2θ) at 11.8, 9.3, 19.9, 22.9, 17.2, 10.2, 20.4, 21.3, 14.2, 18.2, 20.7, 15.4, 24.4, 20.2, 23.7, 15.3, 25.2, 18.7, 18.5, 16.9, and 13.8.

In some embodiments, the crystalline form is Form B, and the XRPD pattern has a peak (±0.2 degrees 2θ) at 11.7.

In some embodiments, the crystalline form is Form B, and the XRPD pattern has peaks (±0.2 degrees 2θ) at 11.7, and 19.9.

In some embodiments, the crystalline form is Form B, and the XRPD pattern has peaks (±0.2 degrees 2θ) at 11.7, 19.9, and 9.3.

In some embodiments, the crystalline form is Form B, and the XRPD pattern has peaks (±0.2 degrees 2θ) at 11.7, 19.9, and 9.3.

In some embodiments, the crystalline form is Form B, and the XRPD pattern has peaks (±0.2 degrees 2θ) at 11.7, 19.9, 9.3, and 10.1.

In some embodiments, the crystalline form is Form B, and the XRPD pattern has peaks (±0.2 degrees 2θ) at 11.7, 19.9, 9.3, 10.1, and 22.9.

In some embodiments, the crystalline form is Form B, and the XRPD pattern has peaks (±0.2 degrees 2θ) at 11.7, 19.9, 9.3, 10.1, 22.9, and 17.2.

In some embodiments, the crystalline form is Form B, and the XRPD pattern has peaks (±0.2 degrees 2θ) at 11.7, 19.9, 9.3, 10.1, 22.9, 17.2, and 20.4.

In some embodiments, the crystalline form is Form B, and the XRPD pattern has peaks (±0.2 degrees 2θ) at 11.7, 19.9, 9.3, 10.1, 22.9, 17.2, 20.4, and 14.2.

In some embodiments, the crystalline form is Form B, and the XRPD pattern has peaks (±0.2 degrees 2θ) at 11.7, 19.9, 9.3, 10.1, 22.9, 17.2, 20.4, 14.2, and 21.3.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, and 18.2.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, 18.2, and 25.5.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, 18.2, 25.5, and 15.4.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, 18.2, 25.5, 15.4, and 20.7.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, 18.2, 25.5, 15.4, 20.7, and 27.6.

In some embodiments, the crystalline form is Form B, and the XRPD pattern has peaks (±0.2 degrees 2θ) at 11.7, 19.9, 9.3, 10.1, 22.9, 17.2, 20.4, 14.2, 21.3, 18.2, 25.5, 15.4, 20.7, 27.6, and 24.4.

Variable humidity XRPD of Form B showed a reversible shift of peaks when the humidity was lowered from 30% RH to 0% RH and back to 30% RH. The XRPD pattern of Form B remained almost unchanged between 30% RH and 90% RH. Only slight differences in XRPD patterns were observed when Form B was exposed to low humidity conditions, however, these patterns can also be explained by the crystal structure (hta02a) of Form B. It is suggested that the dehydrated form is likely an isostructural or isomorphic phase of Form B.

The skilled artisan will recognize that the relative intensities of X-ray powder diffraction pattern peaks can vary depending on the sample preparation technique, crystal size distribution, various filters used, sample mounting procedure, and the particular instrument employed. Accordingly, depending on the type of the instrument and settings employed (including filters), new peaks may be observed in subsequently obtained patterns; or peaks observed in previously obtained patterns may have a negligible relative intensities (and hence may not be observed) in subsequently obtained patterns. As such, the absence of one or more of the lower relative intensity peaks described above may not, in and of itself, establish that Form B is not present (e.g., in a sample). However, the presence of the lower relative intensity peaks described above can, in general, be used to further establish the presence of Form B in a sample.

Form B can also have one or more of the following characteristics.

In some embodiments, the crystalline form is Form B having a TGA curve characterized by a weight loss of about 0.5% to about 10% (e.g., about 0.5% to about 8%, about 0.5% to about 6%, about 0.5% to about 4%, about 0.5% to about 3%, about 0.5% to about 2%, about 0.5% to about 1.5%, or about 1% loss) over about 60° C. to about 100° C. In some embodiments, the crystalline form is Form B having a TGA curve characterized by a weight loss of about 1% over about 60° C. to about 100° C.

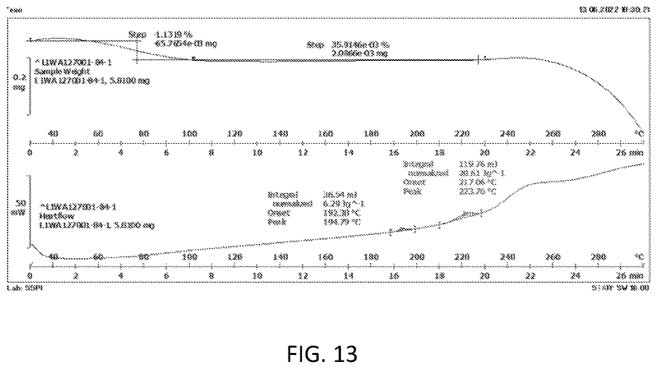

In some embodiments, the crystalline form is Form B having a TGA curve that is substantially the same as that shown in FIG. 13.

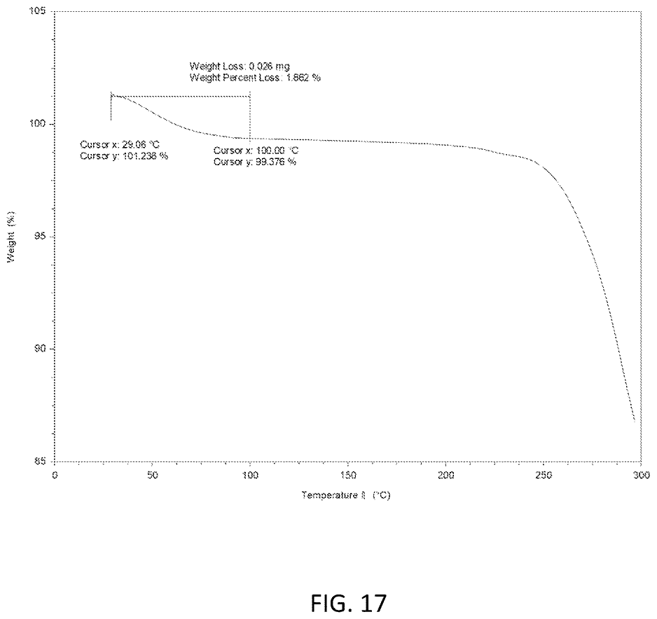

In some embodiments, the crystalline form is Form B having a TGA curve that is substantially the same as that shown in FIG. 17.

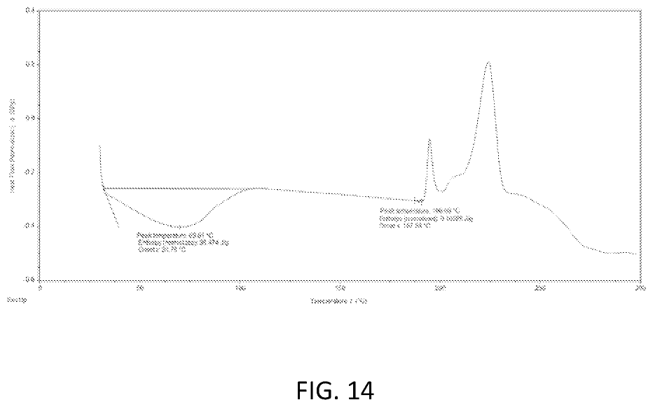

In some embodiments, the crystalline form is Form B having a DSC curve that is substantially the same as that shown in FIG. 14.

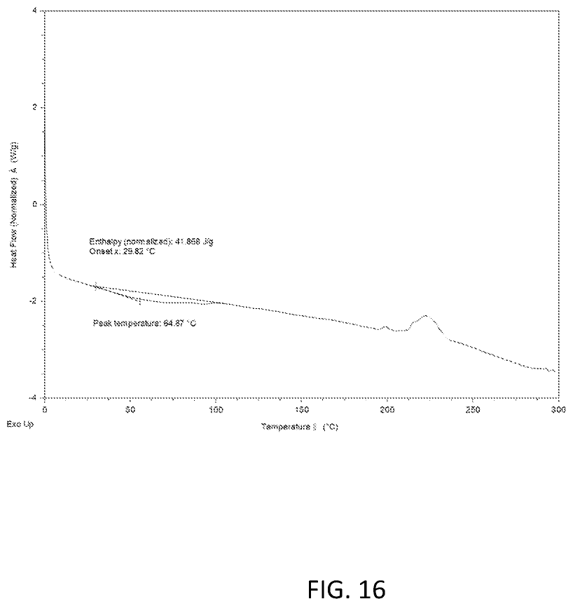

In some embodiments, the crystalline form is Form B having a DSC curve that is substantially the same as that shown in FIG. 16.

In some embodiments, Form B has a DSC thermogram characterized by a broad endotherm at Tₒₙₛₑₜ=29.8° C. and Tₚₑₐₖ=65° C. when heated from 30 and 300° C. at a rate of 10 K/min.

In some embodiments, Form B has a DSC thermogram when heated from 30 and 300° C. at a rate of 10 K/min which shows a broad endotherms at Tₒₙₛₑₜ=29.8° C. with an enthalpy of 41.9 J/g and Tₚₑₐₖ=65° C.

In some embodiments, Form B has a TGA diagram when heated from 30° C. and 300° C. at 10 K/min showing a weight loss of about 1.9% to 2.0% at 100° C.

In some embodiments, Form B absorbs up to 2.7% of moisture at 95% RH at 25° C. by DVS. In some embodiments, the crystalline form is Form B, in substantially pure form. In some embodiments, the crystalline form is Form B, wherein the Form B is at least 99%, 99.3%, 99.5%, 99.7%, or 99.9% pure (w/w). In some embodiments, the crystalline form is Form B, wherein the Form B is at least 99%, 99.3%, 99.5%, 99.7%, or 99.9% pure (w/w) after 1 week of exposure to about 10% relative humidity at about 50° C. In some embodiments, the crystalline form is Form B, wherein the Form B is at least 99%, 99.3%, 99.5%, 99.7%, or 99.9% pure (w/w) after 1 week of exposure to about 10% relative humidity at about 80° C. In some embodiments, the crystalline form is Form B, wherein the Form B is at least 99%, 99.3%, 99.5%, 99.7%, or 99.9% pure (w/w) after 1 week of exposure to about 75% relative humidity at about 50° C. In some embodiments, the crystalline form is Form B, wherein the Form B is at least 99%, 99.3%, 99.5%, 99.7%, or 99.9% pure (w/w) after 1 week of exposure to about 75% relative humidity at about 80° C.

In some embodiments, the crystalline form is Form B characterized by having a solubility of about 0.32 mg/mL to about 0.45 mg/mL in Fasted State Simulated Gastric Fluid (FaSSGF) at about 37° C. after about 30 minutes, for example, about 0.32 mg/mL about 0.33 mg/mL, about 0.34 mg/mL, about 0.35 mg/mL, about 0.36 mg/mL, about 0.37 mg/mL, about 0.38 mg/mL, about 0.39 mg/mL, about 0.40 mg/mL, about 0.41 mg/mL, about 0.42 mg/mL, about 0.43 mg/mL, about 0.44 mg/mL, about 0.45 mg/mL, or any value in between. In some embodiments, Form B is characterized by having a solubility of about 0.36 mg/mL to about 0.42 mg/mL in Fasted State Simulated Gastric Fluid (FaSSGF) at about 37° C. after about 30 minutes. In some embodiments, the crystalline form is Form B characterized by having a solubility of 0.39 mg/mL to 0.41 mg/mL in Fasted State Simulated Gastric Fluid (FaSSGF) at about 37° C. after about 30 minutes. In some embodiments, the crystalline form is Form B characterized by having a solubility of about 0.40 mg/mL in Fasted State Simulated Gastric Fluid (FaSSGF) at about 37° C. after about 30 minutes.

In some embodiments, the crystalline form is Form B characterized by having a solubility of about 0.49 mg/mL to about 0.59 mg/mL in Fasted State Simulated Gastric Fluid (FaSSGF) at about 37° C. after about 24 hours, for example, about 0.49 mg/mL, about 0.50 mg/mL, about 0.51 mg/mL, about 0.52 mg/mL, about 0.53 mg/mL, about 0.54 mg/mL, about 0.55 mg/mL, about 0.56 mg/mL, about 0.57 mg/mL, about 0.58 mg/mL, about 0.59 mg/mL, or any value in between. In some embodiments, Form B is characterized by having a solubility of about 0.50 mg/mL to about 0.56 mg/mL in Fasted State Simulated Gastric Fluid (FaSSGF) at about 37° C. after about 24 hours. In some embodiments, the crystalline form is Form B characterized by having a solubility of 0.53 mg/mL to 0.55 mg/mL in Fasted State Simulated Gastric Fluid (FaSSGF) at about 37° C. after about 24 hours. In some embodiments, the crystalline form is Form B characterized by having a solubility of about 0.54 mg/mL in Fasted State Simulated Gastric Fluid (FaSSGF) at about 37° C. after about 24 hours.

In some embodiments, the crystalline form is Form B characterized by having a solubility of about 0.45 mg/mL to about 0.52 mg/mL in Fasted State Simulated Gastric Fluid (FaSSGF) at an initial pH of 1.6 at about 25° C. after about 24 hours, for example, about 0.49 mg/mL.

In some embodiments, the crystalline form is Form B characterized by having a solubility of greater than about 1.5 mg/mL to greater than about 2 mg/mL in Fed State Simulated Intestinal Fluid (FeSSIF) at about 37° C. after about 30 minutes, for example, greater than about 1.6 mg/mL, greater than about 1.7 mg/mL, greater than about 1.8 mg/mL, greater than about 1.9 mg/mL, or greater than about 2 mg/mL. In some embodiments, the crystalline form is Form B characterized by having a solubility of greater than 1.9 mg/mL to greater than 2 1.9 mg/mL in Fed State Simulated Intestinal Fluid (FeSSIF) at about 37° C. after about 30 minutes. In some embodiments, the crystalline form is Form B characterized by having a solubility of greater than about 2 mg/mL in Fed State Simulated Intestinal Fluid (FeSSIF) at about 37° C. after about 30 minutes.

In some embodiments, the crystalline form is Form B characterized by having a solubility of greater than about 1.5 mg/mL to greater than about 2 mg/mL in Fed State Simulated Intestinal Fluid (FeSSIF) at about 37° C. after about 24 hours, for example, greater than about 1.6 mg/mL, greater than about 1.7 mg/mL, greater than about 1.8 mg/mL, greater than about 1.9 mg/mL, or greater than about 2 mg/mL. In some embodiments, the crystalline form is Form B characterized by having a solubility of greater than 1.9 mg/mL to greater than 2 mg/mL in Fed State Simulated Intestinal Fluid (FeSSIF) at about 37° C. after about 24 hours. In some embodiments, the crystalline form is Form B characterized by having a solubility of greater than about 2 mg/mL in Fed State Simulated Intestinal Fluid (FeSSIF) at about 37° C. after about 24 hours.

In some embodiments, the crystalline form is Form B characterized by having a solubility of greater than about 1.5 mg/mL to greater than about 2 mg/mL in Fasted State Simulated Intestinal Fluid (FaSSIF) at about 37° C. after about 30 minutes, for example, greater than about 1.5 mg/mL, greater than about 1.6 mg/mL, greater than about 1.7 mg/mL, greater than about 1.8 mg/mL, greater than about 1.9 mg/mL, or greater than about 2 mg/mL. In some embodiments, Form B is characterized by having a solubility of greater than 1.8 mg/mL to greater than 1.9 mg/mL in Fasted State Simulated Intestinal Fluid (FaSSIF) at about 37° C. after about 30 minutes. In some embodiments, Form B is characterized by having a solubility of greater than about 2 mg/mL in Fasted State Simulated Intestinal Fluid (FaSSIF) at about 37° C. after about 30 minutes.

In some embodiments, the crystalline form is Form B characterized by having a solubility of about 0.48 mg/mL to about 0.58 mg/mL in Fasted State Simulated Intestinal Fluid (FaSSIF) at about 37° C. after about 24 hours, for example, about 0.48 mg/mL, about 0.49 mg/mL, about 0.50 mg/mL, about 0.51 mg/mL, about 0.52 mg/mL, about 0.53 mg/mL, about 0.54 mg/mL, about 0.55 mg/mL, about 0.56 mg/mL, about 0.57 mg/mL, about 0.58 mg/mL, or any value in between. In some embodiments, Form B is characterized by having a solubility of about 0.51 mg/mL to about 0.54 mg/mL in Fasted State Simulated Intestinal Fluid (FaSSIF) at about 37° C. after about 24 hours. In some embodiments, Form B is characterized by having a solubility of 0.52 mg/mL to 0.54 mg/mL in Fasted State Simulated Intestinal Fluid (FaSSIF) at about 37° C. after about 24 hours. In some embodiments, the crystalline form is Form B characterized by having a solubility of about 0.53 mg/mL in Fasted State Simulated Intestinal Fluid (FaSSIF) at about 37° C. after about 24 hours.

In some embodiments, the crystalline form is Form B characterized by having a solubility of greater than 2 mg/mL in Fasted State Simulated Intestinal Fluid (FaSSIF) at an initial pH of 6.5 at about 25° C. after about 24 hours. In some embodiments, the Fasted State Simulated Intestinal Fluid (FaSSIF) comprises a phosphate buffer saline (PBS).

In some embodiments, the crystalline form is Form B characterized by having a solubility of greater than 2 mg/mL in a phosphate buffer saline (PBS) at an initial pH of 6.5 at about 25° C. after about 24 hours. In some embodiments, a phosphate buffer saline (PBS) comprises sodium taurocholate (NaTC). In some embodiments, a phosphate buffer saline (PBS) comprises sodium taurocholate (NaTC) at 3 mM.

In some embodiments, the crystalline form is Form B characterized by having a solubility of about 0.42 mg/mL to about 0.51 mg/mL in water with an initial pH of about 7.0 at about 37° C. after about 30 minutes, for example, about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, about 0.50 mg/mL, about 0.51 mg/mL, or any value in between. In some embodiments, Form B is characterized by having a solubility of about 0.46 mg/mL to about 0.50 mg/mL in water with an initial pH of about 7.0 at about 37° C. after about 30 minutes. In some embodiments, Form B is characterized by having a solubility of 0.47 mg/mL to 0.49 mg/mL in water with an initial pH of about 7.0 at about 37° C. after about 30 minutes. In some embodiments, the crystalline form is Form B characterized by having a solubility of about 0.48 mg/mL in water with an initial pH of about 7.0 at about 37° C. after about 30 minutes.

In some embodiments, the crystalline form is Form B characterized by having a solubility of about 0.51 mg/mL to about 0.61 mg/mL in water at about 37° C. after about 24 hours, for example, about 0.51 mg/mL, about 0.52 mg/mL, about 0.53 mg/mL, about 0.54 mg/mL, about 0.55 mg/mL, about 0.56 mg/mL, about 0.57 mg/mL, about 0.58 mg/mL, about 0.59 mg/mL, about 0.60 mg/mL, about 0.61 mg/mL, or any value in between. In some embodiments, Form B is characterized by having a solubility of about 0.53 mg/mL to about 0.58 mg/mL in water with an initial pH of about 7.0 at about 37° C. after about 24 hours. In some embodiments, Form B is characterized by having a solubility of 0.55 mg/mL to 0.57 mg/mL in water with an initial pH of about 7.0 at about 37° C. after about 24 hours. In some embodiments, the crystalline form is Form B characterized by having a solubility of about 0.56 mg/mL in water at about 37° C. after about 24 hours.

In some embodiments, the crystalline form is Form B characterized by having a solubility of about 0.38 mg/mL to about 0.42 mg/mL in water with an initial pH of about 7.0 at about 25° C. after 24 hrs, for example, about 0.39 mg/mL.

In some embodiments, the crystalline form is Form B characterized by having an intrinsic dissolution rate of about 0.15 mg/min/cm² to about 0.2 mg/min/cm² in a buffer at pH 6.5, for example 0.18 mg/min/cm². In some embodiments, the buffer is a phosphate buffer.

### Form C

Some embodiments provide a crystalline p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid. In some embodiments, the crystalline p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid is an anhydrate form. In some embodiments, the crystalline form is characterized by an XRPD pattern having a peak at 22.3±0.2 degrees 2θ. For ease of exposition, the aforementioned polymorph is referred to herein as “Form C.” The X-ray powder diffraction pattern of Form C can also include one or more additional characteristic peaks.

In some embodiments, the crystalline form is Form C, and the XRPD pattern is represented by peaks shown in the table below:

| Angle | d Value | | Rel. Intensity | Rel. Intensity Rank |
|---|---|---|---|---|
| 5.372° | 16.43733 | Å | 12.7% | 15 |
| 9.526° | 9.27687 | Å | 9.9% | 16 |
| 10.252° | 8.62154 | Å | 37.3% | 7 |
| 10.700° | 8.26175 | Å | 25.3% | 10 |
| 11.570° | 7.64197 | Å | 53.4% | 5 |
| 13.290° | 6.65652 | Å | 14.6% | 14 |
| 15.324° | 5.77747 | Å | 41.8% | 6 |
| 17.348° | 5.10763 | Å | 74.0% | 2 |
| 17.519° | 5.05816 | Å | 74.0% | 3 |
| 19.856° | 4.46775 | Å | 30.1% | 8 |
| 20.459° | 4.33754 | Å | 18.0% | 11 |
| 21.803° | 4.07299 | Å | 60.2% | 4 |
| 22.294° | 3.98446 | Å | 100.0% | 1 |
| 24.742° | 3.59547 | Å | 16.5% | 12 |
| 25.622° | 3.47395 | Å | 15.6% | 13 |
| 26.698° | 3.33634 | Å | 29.1% | 9 |

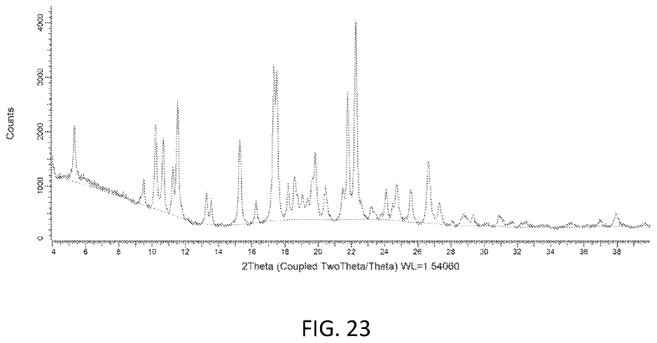

In some embodiments, the crystalline form is Form C, and the XRPD pattern is substantially the same as that shown in FIG. 23.

In some embodiments, the X-ray powder diffraction pattern of Form C can also include one or more of the following additional characteristic peaks, which can also be used to identify Form C (e.g., in a sample).

For example, the XRPD pattern has a peak at 17.3±0.2 degrees 2θ.

For example, the XRPD pattern has a peak at 17.5±0.2 degrees 2θ.

For example, the XRPD pattern has a peak at 21.8±0.2 degrees 2θ.

For example, the XRPD pattern has a peak at 11.5±0.2 degrees 2θ.

For example, the XRPD pattern has a peak at 15.3±0.2 degrees 2θ.

For example, the XRPD pattern has a peak at 10.2±0.2 degrees 2θ.

For example, the XRPD pattern has a peak at 10.7±0.2 degrees 2θ.

For example, the XRPD pattern has a peak at 26.6±0.2 degrees 2θ.

The X-ray powder diffraction pattern of Form C can also include one or more lower intensity characteristic peaks. The relative intensities of these additional peak(s) are, in general, lower than the relative intensities associated with the characteristic peaks described above.

For example, the XRPD pattern has a peak at 19.8±0.2 degrees 2θ.

For example, the XRPD pattern has a peak at 5.3±0.2 degrees 2θ.

For example, the XRPD pattern has a peak at 11.3±0.2 degrees 2θ.

For example, the XRPD pattern has a peak at 19.7±0.2 degrees 2θ.

For example, the XRPD pattern has a peak at 18.6±0.2 degrees 2θ.

For example, the XRPD pattern has a peak at 24.7±0.2 degrees 2θ.

For example, the XRPD pattern has a peak at 24.1±0.2 degrees 2θ.

For example, the XRPD pattern has a peak at 20.4±0.2 degrees 2θ.

For example, the XRPD pattern has a peak at 18.2±0.2 degrees 2θ.

For example, the XRPD pattern has a peak at 25.6±0.2 degrees 2θ.

For example, the XRPD pattern has a peak at 21.5±0.2 degrees 2θ.

For example, the XRPD pattern has a peak at 13.3±0.2 degrees 2θ.

For example, the XRPD pattern has a peak at 19.1±0.2 degrees 2θ.

For example, the XRPD pattern has a peak at 9.5±0.2 degrees 2θ.

For example, the XRPD pattern has a peak at 13.6±0.2 degrees 2θ.

For example, the XRPD pattern has a peak at 27.3±0.2 degrees 2θ.

For example, the XRPD pattern has a peak at 19.4±0.2 degrees 2θ.

For example, the XRPD pattern has a peak at 16.3±0.2 degrees 2θ.

For example, the XRPD pattern has a peak at 37.9±0.2 degrees 2θ.

For example, the XRPD pattern has a peak at 24.5±0.2 degrees 2θ.

For example, the XRPD pattern has a peak at 22.6±0.2 degrees 2θ.

For example, the XRPD pattern has a peak at 39.6±0.2 degrees 2θ.

For example, the XRPD pattern has a peak at 23.2±0.2 degrees 2θ.

For example, the XRPD pattern has a peak at 37.0±0.2 degrees 2θ.

For example, the XRPD pattern has a peak at 28.8±0.2 degrees 2θ.

For example, the XRPD pattern has a peak at 30.9±0.2 degrees 2θ.

For example, the XRPD pattern has a peak at 29.3±0.2 degrees 2θ.

In some embodiments, the crystalline form is Form C, and the XRPD pattern has peaks (±0.2 degrees 2θ) at 22.3, 17.3, and 17.5.

In some embodiments, the crystalline form is Form C, and the XRPD pattern has peaks (±0.2 degrees 2θ) at 22.3, 17.3, 17.5, and 21.8.

In some embodiments, the crystalline form is Form C, and the XRPD pattern has peaks (±0.2 degrees 2θ) at 22.3, 17.3, 17.5, 21.8, 11.5, 15.3, 10.2, 10.7, and 26.6.

In some embodiments, the crystalline form is Form C, and the XRPD pattern has peaks (±0.2 degrees 2θ) at 22.3, 17.3, 17.5, 21.8, 11.5, 15.3, 10.2, 10.7, 26.6, 19.8, 5.3, 11.3, 19.7, 18.6, 24.7, 24.1, 20.4, 18.2, 25.6, 21.5, 13.3, 19.1, 9.5, 13.6, 27.3, 19.4, 16.3, 37.9, 24.5, 22.6, 39.6, 23.2, 37.0, 28.8, 30.9, and 29.3.

The skilled artisan will recognize that the relative intensities of X-ray powder diffraction pattern peaks can vary depending on the sample preparation technique, crystal size distribution, various filters used, sample mounting procedure, and the particular instrument employed. Accordingly, depending on the type of the instrument and settings employed (including filters), new peaks may be observed in subsequently obtained patterns; or peaks observed in previously obtained patterns may have a negligible relative intensities (and hence may not be observed) in subsequently obtained patterns. As such, the absence of one or more of the lower relative intensity peaks described above may not, in and of itself, establish that Form C is not present (e.g., in a sample). However, the presence of the lower relative intensity peaks described above can, in general, be used to further establish the presence of Form C in a sample.

Form C can also have one or more of the following characteristics.

In some embodiments, the crystalline form is Form C having a TGA curve characterized by a weight loss of about 0.1% to about 10% (e.g., about 0.1% to about 7%, about 0.1% to about 4%, about 0.1% to about 2%, about 0.1% to about 1%, about 0.2% to about 0.6%, or about 0.4%) from about 210 to about 240° C. In some embodiments, the crystalline form is Form C having a TGA curve characterized by a weight loss of about 0.4% from about 210 to about 240° C.

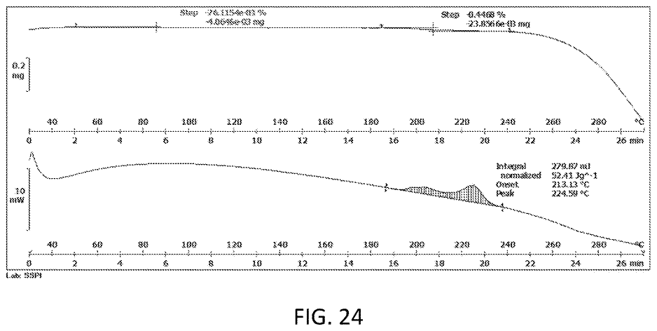

In some embodiments, the crystalline form is Form C having a TGA curve that is substantially the same as that shown in FIG. 24.

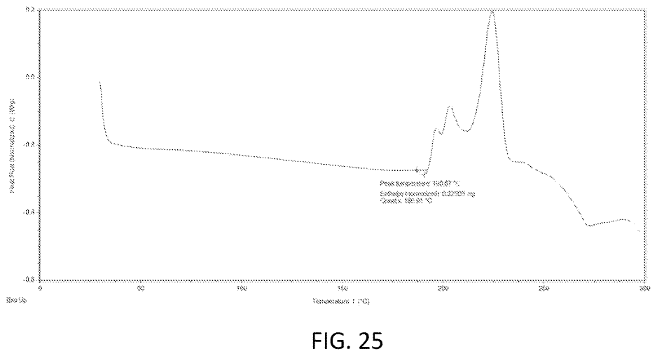

In some embodiments, the crystalline form is Form C having a DSC curve that is substantially the same as that shown in FIG. 25. In some embodiments, the crystalline form is Form C having a DSC curve characterized by a melting onset of about 187° C.

In some embodiments, the crystalline form is Form C, in substantially pure form. In some embodiments, the crystalline form is Form C, wherein the Form C is at least 99%, 99.3%, 99.5%, 99.7%, or 99.9% pure (w/w). In some embodiments, the crystalline form is Form C, wherein the Form C is at least 99%, 99.3%, 99.5%, 99.7%, or 99.9% pure (w/w) after 1 week of exposure to about 10% relative humidity at about 50° C. In some embodiments, the crystalline form is Form C, wherein the Form C is at least 99%, 99.3%, 99.5%, 99.7%, or 99.9% pure (w/w) after 1 week of exposure to about 10% relative humidity at about 80° C. In some embodiments, the crystalline form is Form C, wherein the Form C is at least 99%, 99.3%, 99.5%, 99.7%, or 99.9% pure (w/w) after 1 week of exposure to about 75% relative humidity at about 50° C. In some embodiments, the crystalline form is Form C, wherein the Form C is at least 99%, 99.3%, 99.5%, 99.7%, or 99.9% pure (w/w) after 1 week of exposure to about 75% relative humidity at about 80° C.

In some embodiments, Form C has a TGA diagram when heated from 30° C. and 300° C. at 10 K/min showing a weight loss of about 0.2% at 180° C.

In some embodiments, Form C absorbs up to 0.4% of moisture at 95% RH at 25° C. by DVS.

In some embodiments, the crystalline form is Form C characterized by having a solubility of about 0.82 mg/mL to about 0.91 mg/mL in Fasted State Simulated Gastric Fluid (FaSSGF) at about 37° C. after about 30 minutes, for example, about 0.82 mg/mL, about 0.83 mg/mL, about 0.84 mg/mL, about 0.85 mg/mL, about 0.86 mg/mL, about 0.87 mg/mL, about 0.88 mg/mL, about 0.89 mg/mL, about 0.90 mg/mL, about 0.91 mg/mL, or any value in between. In some embodiments, Form C is characterized by having a solubility of about 0.85 mg/mL to about 0.89 mg/mL in Fasted State Simulated Gastric Fluid (FaSSGF) at about 37° C. after about 30 minutes. In some embodiments, Form C is characterized by having a solubility of 0.86 mg/mL to 0.88 mg/mL in Fasted State Simulated Gastric Fluid (FaSSGF) at about 37° C. after about 30 minutes. In some embodiments, Form C is characterized by having a solubility of about 0.87 mg/mL in Fasted State Simulated Gastric Fluid (FaSSGF) at about 37° C. after about 30 minutes. In some embodiments, the crystalline form is Form C characterized by having a solubility of about 0.8 mg/mL to about 0.9 mg/mL in Fasted State Simulated Gastric Fluid (FaSSGF) at about 37° C. after about 24 hours, for example, about 0.8 mg/mL, about 0.81 mg/mL, about 0.82 mg/mL, about 0.83 mg/mL, about 0.84 mg/mL, about 0.85 mg/mL, about 0.86 mg/mL, about 0.87 mg/mL, about 0.88 mg/mL, about 0.89 mg/mL, about 0.90 mg/mL, or any value in between. In some embodiments, Form C is characterized by having a solubility of about 0.83 mg/mL to about 0.88 mg/mL in Fasted State Simulated Gastric Fluid (FaSSGF) at about 37° C. after about 24 hours. In some embodiments, Form C is characterized by having a solubility of 0.84 mg/mL to 0.87 mg/mL in Fasted State Simulated Gastric Fluid (FaSSGF) at about 37° C. after about 24 hours. In some embodiments, the crystalline form is Form C characterized by having a solubility of about 0.85 mg/mL in Fasted State Simulated Gastric Fluid (FaSSGF) at about 37° C. after about 24 hours. In some embodiments, the crystalline form is Form C characterized by having a solubility of about 1.58 mg/mL to about 1.70 mg/mL in Fed State Simulated Intestinal Fluid (FeSSIF) at about 37° C. after about 30 minutes, for example, about 1.58 mg/mL, about 1.59 mg/mL, about 1.60 mg/mL, about 1.61 mg/mL, about 1.62 mg/mL, about 1.63 mg/mL, about 1.64 mg/mL, about 1.65 mg/mL, about 1.66 mg/mL, about 1.67 mg/mL, about 1.68 mg/mL, about 1.69 mg/mL, about 1.70 mg/mL, or any value in between. In some embodiments, the crystalline form is Form C characterized by having a solubility of about 1.60 mg/mL to about 1.65 mg/mL in Fed State Simulated Intestinal Fluid (FeSSIF) at about 37° C. after about 30 minutes. In some embodiments, the crystalline form is Form C characterized by having a solubility of 1.62 mg/mL to 1.64 mg/mL in Fed State Simulated Intestinal Fluid (FeSSIF) at about 37° C. after about 30 minutes. In some embodiments, the crystalline form is Form C characterized by having a solubility of about 1.63 mg/mL in Fed State Simulated Intestinal Fluid (FeSSIF) at about 37° C. after about 30 minutes.

In some embodiments, the crystalline form is Form C characterized by having a solubility of about 1.64 mg/mL to about 1.74 mg/mL in Fed State Simulated Intestinal Fluid (FeSSIF) at about 37° C. after about 24 hours, for example, about 1.64 mg/mL, about 1.65 mg/mL, about 1.66 mg/mL, about 1.67 mg/mL, about 1.68 mg/mL, about 1.69 mg/mL, about 1.70 mg/mL, about 1.71 mg/mL, about 1.72 mg/mL, about 1.73 mg/mL, about 1.74 mg/mL, or any value in between. In some embodiments, the crystalline form is Form C characterized by having a solubility of about 1.67 mg/mL to about 1.71 mg/mL in Fed State Simulated Intestinal Fluid (FeSSIF) at about 37° C. after about 24 hours. In some embodiments, the crystalline form is Form C characterized by having a solubility of 1.68 mg/mL to 1.70 mg/mL in Fed State Simulated Intestinal Fluid (FeSSIF) at about 37° C. after about 24 hours. In some embodiments, the crystalline form is Form C characterized by having a solubility of about 1.69 mg/mL in Fed State Simulated Intestinal Fluid (FeSSIF) at about 37° C. after about 24 hours.

In some embodiments, the crystalline form is Form C characterized by having a solubility of about 1.62 mg/mL to about 1.72 mg/mL in Fasted State Simulated Intestinal Fluid (FaSSIF) at about 37° C. after about 30 minutes, for example, about 1.62 mg/mL, about 1.63 mg/mL, about 1.64 mg/mL, about 1.65 mg/mL, about 1.66 mg/mL, about 1.67 mg/mL, about 1.68 mg/mL, about 1.69 mg/mL, about 1.70 mg/mL, about 1.71 mg/mL, or any value in between. In some embodiments, the crystalline form is Form C characterized by having a solubility of about 1.66 mg/mL to about 1.70 mg/mL in Fasted State Simulated Intestinal Fluid (FaSSIF) at about 37° C. after about 30 minutes. In some embodiments, the crystalline form is Form C characterized by having a solubility of 1.67 mg/mL to 1.69 mg/mL in Fasted State Simulated Intestinal Fluid (FaSSIF) at about 37° C. after about 30 minutes. In some embodiments, the crystalline form is Form C characterized by having a solubility of about 1.68 mg/mL in Fasted State Simulated Intestinal Fluid (FaSSIF) at about 37° C. after about 30 minutes.

In some embodiments, the crystalline form is Form C characterized by having a solubility of about 0.08 mg/mL to about 0.18 mg/mL in Fasted State Simulated Intestinal Fluid (FaSSIF) at about 37° C. after about 24 hours, for example, about 0.08 mg/mL, about 0.09 mg/mL, about 0.10 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, or any value in between. In some embodiments, the crystalline form is Form C characterized by having a solubility of about 0.11 mg/mL to about 0.15 mg/mL in Fasted State Simulated Intestinal Fluid (FaSSIF) at about 37° C. after about 24 hours. In some embodiments, the crystalline form is Form C characterized by having a solubility of 0.12 mg/mL to 0.14 mg/mL in Fasted State Simulated Intestinal Fluid (FaSSIF) at about 37° C. after about 24 hours. In some embodiments, the crystalline form is Form C characterized by having a solubility of about 0.13 mg/mL in Fasted State Simulated Intestinal Fluid (FaSSIF) at about 37° C. after about 24 hours. In some embodiments, the crystalline form is Form C characterized by having a solubility of about 0.82 mg/mL to about 0.92 mg/mL in water with an initial pH of about 7.0 at about 37° C. after about 30 minutes, for example, about 0.82 mg/mL, about 0.83 mg/mL, about 0.84 mg/mL, about 0.85 mg/mL, about 0.86 mg/mL, about 0.87 mg/mL, about 0.88 mg/mL, about 0.89 mg/mL, about 0.90 mg/mL, about 0.91 mg/mL, about 0.92 mg/mL, or any value in between. In some embodiments, Form C is characterized by having a solubility of about 0.86 mg/mL to about 0.90 mg/mL in water with an initial pH of about 7.0 at about 37° C. after about 30 minutes. In some embodiments, Form C is characterized by having a solubility of 0.87 mg/mL to 0.89 mg/mL in water with an initial pH of about 7.0 at about 37° C. after about 30 minutes. In some embodiments, the crystalline form is Form C characterized by having a solubility of about 0.88 mg/mL in water with an initial pH of about 7.0 at about 37° C. after about 30 minutes.

In some embodiments, the crystalline form is Form C characterized by having a solubility of about 0.8 mg/mL to about 1.4 mg/mL in water with an initial pH of about 7.0 at about 37° C. after about 24 hours, for example, about 0.8 mg/mL, about 0.85 mg/mL, about 0.9 mg/mL, about 0.95 mg/mL, about 1.0 mg/mL, about 1.05 mg/mL, about 1.1 mg/mL, about 1.15 mg/mL, about 1.2 mg/mL, about 1.25 mg/mL, about 1.3 mg/mL, about 1.35 mg/mL, about 1.4 mg/mL, or any value in between. In some embodiments, Form C is characterized by having a solubility of about 0.86 mg/mL to about 0.90 mg/mL in water with an initial pH of about 7.0 at about 37° C. after about 24 hours. In some embodiments, Form C is characterized by having a solubility of 1.10 mg/mL to 1.14 mg/mL in water with an initial pH of about 7.0 at about 37° C. after about 24 hours. In some embodiments, the crystalline form is Form C characterized by having a solubility of about 1.12 mg/mL in water with an initial pH of about 7.0 at about 37° C. after about 24 hours.

### Form D

Some embodiments provide a crystalline hydrochloride salt form of (S)-4-(2,2-difluoro-7-((5-methoxy-7-methyl-1H-indol-4-yl)methyl)-7-azaspiro[3.5]nonan-6-yl)benzoic acid.

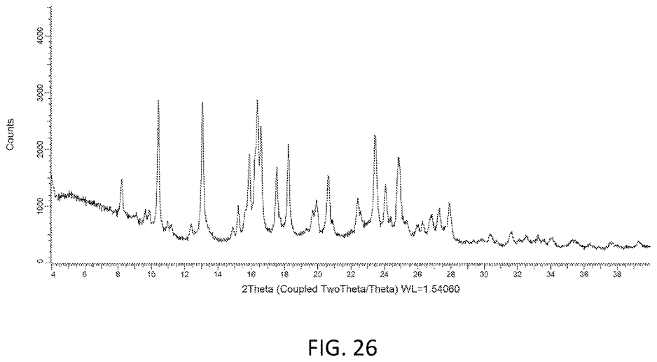

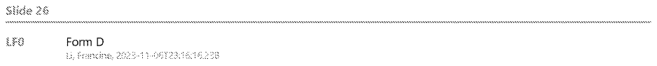

In some embodiments, the crystalline form is Form D, and the XRPD pattern is substantially the same as that shown in FIG. 26.

In some embodiments, the crystalline form is characterized by an XRPD pattern comprising a peak at 13.1±0.2 degrees 2θ. For ease of exposition, the aforementioned polymorph is referred to herein as “Form D.” The X-ray powder diffraction pattern of Form D can also include one or more additional characteristic peaks.

The X-ray powder diffraction pattern of Form D can also include one or more lower intensity characteristic peaks. The relative intensities of these additional peak(s) are, in general, lower than the relative intensities associated with the characteristic peaks described above.

For example, the XRPD pattern has a peak at 16.4±0.2 degrees 2θ.

For example, the XRPD pattern has a peak at 10.4±0.2 degrees 2θ.

For example, the XRPD pattern has a peak at 16.6±0.2 degrees 2θ.

For example, the XRPD pattern has a peak at 23.4±0.2 degrees 2θ.

For example, the XRPD pattern has a peak at 18.2±0.2 degrees 2θ.

For example, the XRPD pattern has a peak at 15.9±0.2 degrees 2θ.

For example, the XRPD pattern has a peak at 24.9±0.2 degrees 2θ.

For example, the XRPD pattern has a peak at 17.5±0.2 degrees 2θ.

The X-ray powder diffraction pattern of Form D can also include one or more lower intensity characteristic peaks. The relative intensities of these additional peak(s) are, in general, lower than the relative intensities associated with the characteristic peaks described above.

For example, the XRPD pattern has a peak at 20.6±0.2 degrees 2θ.

For example, the XRPD pattern has a peak at 24.1±0.2 degrees 2θ.

For example, the XRPD pattern has a peak at 27.9±0.2 degrees 2θ.

For example, the XRPD pattern has a peak at 22.4±0.2 degrees 2θ.

For example, the XRPD pattern has a peak at 8.2±0.2 degrees 2θ.

For example, the XRPD pattern has a peak at 19.9±0.2 degrees 2θ.

For example, the XRPD pattern has a peak at 15.2±0.2 degrees 2θ.

For example, the XRPD pattern has a peak at 27.3±0.2 degrees 2θ.

For example, the XRPD pattern has a peak at 15.7±0.2 degrees 2θ.

For example, the XRPD pattern has a peak at 26.8±0.2 degrees 2θ.

For example, the XRPD pattern has a peak at 19.7±0.2 degrees 2θ.

For example, the XRPD pattern has a peak at 22.6±0.2 degrees 2θ.

For example, the XRPD pattern has a peak at 26.3±0.2 degrees 2θ.

For example, the XRPD pattern has a peak at 25.4±0.2 degrees 2θ.

For example, the XRPD pattern has a peak at 9.9±0.2 degrees 2θ.

For example, the XRPD pattern has a peak at 9.6±0.2 degrees 2θ.

For example, the XRPD pattern has a peak at 24.4±0.2 degrees 2θ.

For example, the XRPD pattern has a peak at 12.4±0.2 degrees 2θ.

For example, the XRPD pattern has a peak at 31.6±0.2 degrees 2θ.

For example, the XRPD pattern has a peak at 20.9±0.2 degrees 2θ.

For example, the XRPD pattern has a peak at 14.9±0.2 degrees 2θ.

For example, the XRPD pattern has a peak at 11.0±0.2 degrees 2θ.

For example, the XRPD pattern has a peak at 33.2±0.2 degrees 2θ.

For example, the XRPD pattern has a peak at 30.3±0.2 degrees 2θ.

For example, the XRPD pattern has a peak at 26.0±0.2 degrees 2θ.

For example, the XRPD pattern has a peak at 11.2±0.2 degrees 2θ.

For example, the XRPD pattern has a peak at 34.0±0.2 degrees 2θ.

For example, the XRPD pattern has a peak at 32.5±0.2 degrees 2θ.

For example, the XRPD pattern has a peak at 39.3±0.2 degrees 2θ.

For example, the XRPD pattern has a peak at 35.3±0.2 degrees 2θ.

For example, the XRPD pattern has a peak at 37.6±0.2 degrees 2θ.

For example, the XRPD pattern has a peak at 35.5±0.2 degrees 2θ.

For example, the XRPD pattern has a peak at 32.1±0.2 degrees 2θ.

For example, the XRPD pattern has a peak at 29.8±0.2 degrees 2θ.

In some embodiments, the crystalline form is Form D, and the XRPD pattern has peaks (±0.2 degrees 2θ) at 13.1, 16.4, and 10.4.

In some embodiments, the crystalline form is Form D, and the XRPD pattern has peaks (±0.2 degrees 2θ) at 13.1, 16.4, 10.4, 16.6, and 23.4.

In some embodiments, the crystalline form is Form D, and the XRPD pattern has peaks (±0.2 degrees 2θ) at 13.1, 16.4, 10.4, 16.6, 23.4, 18.2, 15.9, 24.9, and 17.5.

In some embodiments, the crystalline form is Form D, and the XRPD pattern has peaks (±0.2 degrees 2θ) at 13.1, 16.4, 10.4, 16.6, 23.4, 18.2, 15.9, 24.9, 17.5, 20.6, 24.1, 27.91, 22.4, 8.2, 19.9, 15.2, 27.3, 15.7, 26.8, 19.7, 22.6, 26.3, 25.4, 9.9, 9.6, 24.4, 12.4, 31.6, 20.9, 14.9, 11.0, 33.2, 30.3, 26.0, 11.2, 34.0, 32.5, 39.3, 35.3, 37.6, 35.5, 32.1, and 29.8.

The skilled artisan will recognize that the relative intensities of X-ray powder diffraction pattern peaks can vary depending on the sample preparation technique, crystal size distribution, various filters used, sample mounting procedure, and the particular instrument employed. Accordingly, depending on the type of the instrument and settings employed (including filters), new peaks may be observed in subsequently obtained patterns; or peaks observed in previously obtained patterns may have a negligible relative intensities (and hence may not be observed) in subsequently obtained patterns. As such, the absence of one or more of the lower relative intensity peaks described above may not, in and of itself, establish that Form D is not present (e.g., in a sample). However, the presence of the lower relative intensity peaks described above can, in general, be used to further establish the presence of Form D in a sample.

Form D can also have one or more of the following characteristics.

In some embodiments, the crystalline form is Form D characterized by a TGA curve indicating a weight loss of about 0.1% to about 10% (e.g., about 0.1% to about 5%, about 0.1% to about 4%, about 0.1% to about 3%, about 0.1% to about 2%, about 0.1% about 1.3%, about 0.4% to about 1%, about 0.6% to about 0.8%, or about 0.7%) between about 210 to about 230° C. In some embodiments, the crystalline form is Form D characterized by a TGA curve indicating a weight loss of about 0.7% between about 210 to about 230° C.

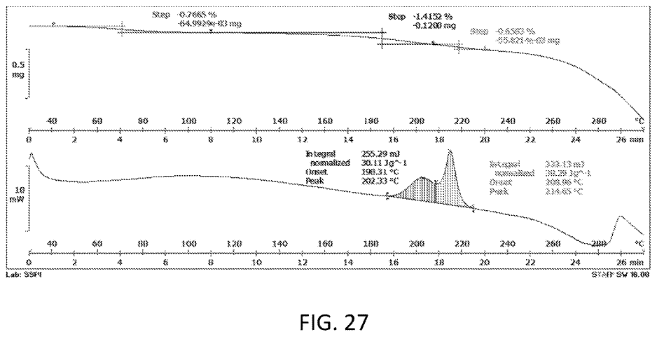

In some embodiments, the crystalline form is Form D characterized by a TGA curve that is substantially the same as that shown in FIG. 27.

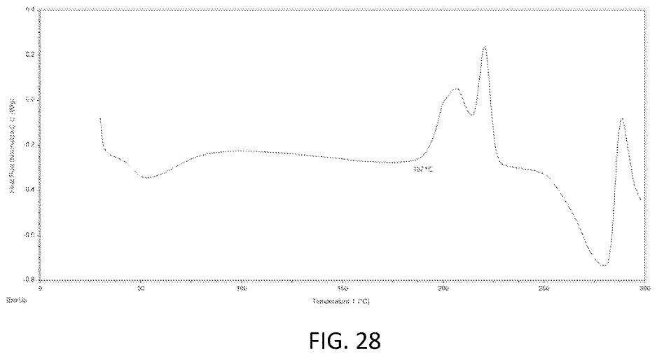

In some embodiments, the crystalline form is Form D characterized by a DSC curve that is substantially the same as that shown in FIG. 28.

In some embodiments, the crystalline form is Form D, in substantially pure form. In some embodiments, the crystalline form is Form D, wherein the Form D is at least 99%, 99.3%, 99.5%, 99.7%, or 99.9% pure (w/w). In some embodiments, the crystalline form is Form D, wherein the Form D is at least 99%, 99.3%, 99.5%, 99.7%, or 99.9% pure (w/w) after 1 week of exposure to about 10% relative humidity at about 50° C. In some embodiments, the crystalline form is Form D, wherein the Form D is at least 99%, 99.3%, 99.5%, 99.7%, or 99.9% pure (w/w) after 1 week of exposure to about 10% relative humidity at about 80° C. In some embodiments, the crystalline form is Form D, wherein the Form D is at least 99%, 99.3%, 99.5%, 99.7%, or 99.9% pure (w/w) after 1 week of exposure to about 75% relative humidity at about 50° C. In some embodiments, the crystalline form is Form D, wherein the Form D is at least 99%, 99.3%, 99.5%, 99.7%, or 99.9% pure (w/w) after 1 week of exposure to about 75% relative humidity at about 80° C.

In some embodiments, the crystalline form is Form D characterized by having a solubility of about 2.2 mg/mL to about 2.3 mg/mL in Fasted State Simulated Gastric Fluid (FaSSGF) at about 37° C. after about 30 minutes, for example, about 2.2 mg/mL, about 2.22 mg/mL, about 2.24 mg/mL, about 2.26 mg/mL, about 2.28 mg/mL, about 2.30 mg/mL, or any value in between. In some embodiments, Form D is characterized by having a solubility of about 2.22 mg/mL to about 2.28 mg/mL in Fasted State Simulated Gastric Fluid (FaSSGF) at about 37° C. after about 30 minutes. In some embodiments, Form D is characterized by having a solubility of 2.24 mg/mL to 2.26 mg/mL in Fasted State Simulated Gastric Fluid (FaSSGF) at about 37° C. after about 30 minutes. In some embodiments, the crystalline form is Form D characterized by having a solubility of about 2.25 mg/mL in Fasted State Simulated Gastric Fluid (FaSSGF) at about 37° C. after about 30 minutes.

In some embodiments, the crystalline form is Form D characterized by having a solubility of greater than about 1.5 mg/mL to greater than about 2 mg/mL in Fasted State Simulated Gastric Fluid (FaSSGF) at about 37° C. after about 24 hours, for example, greater than about 1.6 mg/mL, greater than about 1.7 mg/mL, greater than about 1.8 mg/mL, greater than about 1.9 mg/mL, or greater than about 2 mg/mL. In some embodiments, Form D is characterized by having a solubility of greater than about 1.8 mg/mL to greater than about 2 mg/mL in Fasted State Simulated Gastric Fluid (FaSSGF) at about 37° C. after about 24 hours. In some embodiments, Form D is characterized by having a solubility of greater than 1.9 mg/mL in Fasted State Simulated Gastric Fluid (FaSSGF) at about 37° C. after about 24 hours. In some embodiments, the crystalline form is Form D characterized by having a solubility of greater than about 2 mg/mL in Fasted State Simulated Gastric Fluid (FaSSGF) at about 37° C. after about 24 hours.

In some embodiments, the crystalline form is Form D characterized by having a solubility of about 1.95 mg/mL to about 2.25 in Fed State Simulated Intestinal Fluid (FeSSIF) at about 37° C. after about 30 minutes, for example, about 1.95 mg/mL, about 2.0 mg/mL, about 2.05 mg/mL, about 2.10 mg/mL, about 2.15 mg/mL, about 2.2 mg/mL, about 2.25 mg/mL, or any value in between. In some embodiments, Form D is characterized by having a solubility of about 2.16 mg/mL to about 2.22 mg/mL in Fed State Simulated Intestinal Fluid (FeSSIF) at about 37° C. after about 30 minutes. In some embodiments, Form D is characterized by having a solubility of 2.18 mg/mL to 2.20 mg/mL in Fed State Simulated Intestinal Fluid (FeSSIF) at about 37° C. after about 30 minutes. In some embodiments, the crystalline form is Form D characterized by having a solubility of about 2.19 mg/mL in Fed State Simulated Intestinal Fluid (FeSSIF) at about 37° C. after about 30 minutes.

In some embodiments, the crystalline form is Form D characterized by having a solubility of about 2.28 mg/mL to about 2.38 mg/mL in Fed State Simulated Intestinal Fluid (FeSSIF) at about 37° C. after about 24 hours, for example, about 2.28 mg/mL, about 2.29 mg/mL, about 2.30 mg/mL, about 2.31 mg/mL, about 2.32 mg/mL, about 2.33 mg/mL, about 2.34 mg/mL, about 2.35 mg/mL, about 2.36 mg/mL, about 2.37 mg/mL, about 2.38 mg/mL, or any value in between. In some embodiments, Form D is characterized by having a solubility of about 2.31 mg/mL to about 2.35 mg/mL in Fed State Simulated Intestinal Fluid (FeSSIF) at about 37° C. after about 24 hours. In some embodiments, Form D is characterized by having a solubility of 2.32 mg/mL to 2.34 mg/mL in Fed State Simulated Intestinal Fluid (FeSSIF) at about 37° C. after about 24 hours. In some embodiments, the crystalline form is Form D characterized by having a solubility of about 2.33 mg/mL in Fed State Simulated Intestinal Fluid (FeSSIF) at about 37° C. after about 24 hours.

In some embodiments, the crystalline form is Form D characterized by having a solubility of about 2.14 mg/mL to about 2.24 mg/mL in Fasted State Simulated Intestinal Fluid (FaSSIF) at about 37° C. after about 30 minutes, for example, about 2.14 mg/mL, about 2.15 mg/mL, about 2.16 mg/mL, about 2.17 mg/mL, about 2.18 mg/mL, about 2.19 mg/mL, about 2.20 mg/mL, about 2.21 mg/mL, about 2.22 mg/mL, about 2.23 mg/mL, about 2.24 mg/mL, or any value in between. In some embodiments, Form D is characterized by having a solubility of about 2.16 mg/mL to about 2.22 mg/mL in Fasted State Simulated Intestinal Fluid (FaSSIF) at about 37° C. after about 30 minutes. In some embodiments, Form D is characterized by having a solubility of 2.18 mg/mL to 2.20 mg/mL in Fasted State Simulated Intestinal Fluid (FaSSIF) at about 37° C. after about 30 minutes. In some embodiments, the crystalline form is Form D characterized by having a solubility of about 2.19 mg/mL in Fasted State Simulated Intestinal Fluid (FaSSIF) at about 37° C. after about 30 minutes.

In some embodiments, the crystalline form is Form D characterized by having a solubility of greater than about 1.5 mg/mL to greater than about 2 mg/mL in Fasted State Simulated Intestinal Fluid (FaSSIF) at about 37° C. after about 24 hours, for example, greater than about 1.5 mg/mL, greater than about 1.6 mg/mL, greater than about 1.7 mg/mL, greater than about 1.8 mg/mL, greater than about 1.9 mg/mL, or greater than about 2 mg/mL. In some embodiments, Form D is characterized by having a solubility of greater than about 1.8 mg/mL to greater than about 2 mg/mL in Fasted State Simulated Intestinal Fluid (FaSSIF) at about 37° C. after about 24 hours. In some embodiments, Form D is characterized by having a solubility of greater than 1.9 mg/mL to greater than 2 mg/mL in Fasted State Simulated Intestinal Fluid (FaSSIF) at about 37° C. after about 24 hours. In some embodiments, the crystalline form is Form D characterized by having a solubility of greater than about 2 mg/mL in Fasted State Simulated Intestinal Fluid (FaSSIF) at about 37° C. after about 24 hours.

In some embodiments, the crystalline form is Form D characterized by having a solubility of about 2.03 mg/mL to about 2.13 mg/mL in water with an initial pH of about 7.0 at about 37° C. after about 30 minutes, for example, about 2.03 mg/mL, about 2.04 mg/mL, about 2.05 mg/mL, about 2.06 mg/mL, about 2.07 mg/mL, about 2.08 mg/mL, about 2.09 mg/mL, about 2.10 mg/mL, about 2.11 mg/mL, about 2.12 mg/mL, about 2.13 mg/mL, or any value in between. In some embodiments, Form D is characterized by having a solubility of about 2.06 mg/mL to about 2.10 mg/mL in water with an initial pH of about 7.0 at about 37° C. after about 30 minutes. In some embodiments, Form D is characterized by having a solubility of 2.07 mg/mL to 2.09 mg/mL in water with an initial pH of about 7.0 at about 37° C. after about 30 minutes. In some embodiments, the crystalline form is Form D characterized by having a solubility of about 2.08 mg/mL in water at about 37° C. after about 30 minutes.

In some embodiments, the crystalline form is Form D characterized by having a solubility of greater than about 1.5 mg/mL to greater than about 2 mg/mL in water with an initial pH of about 7.0 at about 37° C. after about 24 hours, for example, greater than about 1.5 mg/mL, greater than about 1.6 mg/mL, greater than about 1.7 mg/mL, greater than about 1.8 mg/mL, greater than about 1.9 mg/mL, or greater than about 2 mg/mL. In some embodiments, Form D is characterized by having a solubility of greater than about 1.8 mg/mL to greater than about 2 mg/mL in water with an initial pH of about 7.0 at about 37° C. after about 24 hours. In some embodiments, Form D is characterized by having a solubility of greater than 1.9 mg/mL to 2 mg/mL in water with an initial pH of about 7.0 at about 37° C. after about 24 hours. In some embodiments, the crystalline form is Form D characterized by having a solubility of greater than about 2 mg/mL in water with an initial pH of about 7.0 at about 37° C. after about 24 hours.

In some embodiments, the crystalline form or the crystalline form (e.g., Form A, Form B, Form C, or Form D) comprises about 1% to about 99% of the corresponding solvate. For example, about 1% to about 5%, about 6% to about 10%, about 10% to about 15%, about 15% to about 20%, about 25% to about 30%, about 30% to about 35%, about 35% to about 40%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99%, about 1% to about 30%, about 30% to about 60%, about 60% to about 99%, about 1%, about 2%, about 3%, about 5%, about 10%, about 12%, about 15%, about 17%, about 20%, about 25%, about 30%, about 35%, about 40%, about 45%, about 50%, about 55%, about 60%, about 65%, about 70%, about 75%, about 80%, about 85%, about 90%, about 95%, or about 99% of the crystalline form comprises the corresponding solvate. In some embodiments, the solvate is a methanol solvate, an ethyl acetate solvate, an ethanol solvate, an isopropanol solvate, a tetrahydrofuran solvate, an acetonitrile solvate, or a diethyl ether solvate. In some embodiments, the solvate is an isopropanol solvate.

### Methods of Preparing Forms A, B, C, and D

As can be appreciated by the skilled artisan, further methods of synthesizing (S)-4-(2,2-difluoro-7-((5-methoxy-7-methyl-1H-indol-4-yl)methyl)-7-azaspiro[3.5]nonan-6-yl)benzoic acid, crystalline forms thereof, and crystalline forms (e.g., tosylate salt forms and hydrochloride salt forms) will be evident to those of ordinary skill in the art. Synthetic chemistry transformations and protecting group methodologies (protection and deprotection) useful in synthesizing (S)-4-(2,2-difluoro-7-((5-methoxy-7-methyl-1H-indol-4-yl)methyl)-7-azaspiro[3.5]nonan-6-yl)benzoic acid are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Methods of Preparing Form A

In some embodiments, the crystalline form is Form A prepared by a method comprising:
(a) adding (S)-4-(2,2-difluoro-7-((5-methoxy-7-methyl-1H-indol-4-yl)methyl)-7-azaspiro[3.5]nonan-6-yl)benzoic acid to an alcohol to form a solution;
(b) cooling the solution to form a suspension;
(c) filtering the suspension to provide the crystalline form.

In some embodiments, the (S)-4-(2,2-difluoro-7-((5-methoxy-7-methyl-1H-indol-4-yl)methyl)-7-azaspiro[3.5]nonan-6-yl)benzoic acid in step (a) is amorphous (S)-4-(2,2-difluoro-7-((5-methoxy-7-methyl-1H-indol-4-yl)methyl)-7-azaspiro[3.5]nonan-6-yl)benzoic acid.

In some embodiments, the alcohol comprises methanol, ethanol, and/or isopropanol. For example, the alcohol comprises isopropanol. In some embodiments, the alcohol is methanol, ethanol, or isopropanol. For example, the alcohol is isopropanol. In some embodiments, adding (S)-4-(2,2-difluoro-7-((5-methoxy-7-methyl-1H-indol-4-yl)methyl)-7-azaspiro[3.5]nonan-6-yl)benzoic acid to an alcohol to form a solution is performed with agitation. In some embodiments, the agitation comprises stirring. In some embodiments, the agitation is stirring. In some embodiments, adding (S)-4-(2,2-difluoro-7-((5-methoxy-7-methyl-1H-indol-4-yl)methyl)-7-azaspiro[3.5]nonan-6-yl)benzoic acid to an alcohol to form a solution is performed at a temperature of about 25° C. to about 70° C. In some embodiments, adding (S)-4-(2,2-difluoro-7-((5-methoxy-7-methyl-1H-indol-4-yl)methyl)-7-azaspiro[3.5]nonan-6-yl)benzoic acid to an alcohol to form a solution is performed at a temperature of about 25° C. to about 50° C., 50° C. to about 70° C., 30° C. to about 60° C., 35° C. to about 55° C., 40° C. to about 50° C., 40° C. to about 45° C., 45° C. to about 50° C. In some embodiments, the concentration of (S)-4-(2,2-difluoro-7-((5-methoxy-7-methyl-1H-indol-4-yl)methyl)-7-azaspiro[3.5]nonan-6-yl)benzoic acid in the solution is about 0.05M to about 0.8M, for example, about 0.05 M to about 0.4 M, about 0.4 M to about 0.8 M, about 0.2 M to about 0.6 M, about 0.4 M to about 0.5 M, about 0.42 M to about 0.4 6M, or about 0.44 M. For example, the concentration of (S)-4-(2,2-difluoro-7-((5-methoxy-7-methyl-1H-indol-4-yl)methyl)-7-azaspiro[3.5]nonan-6-yl)benzoic acid in the solution is about 0.44 M.

In some embodiments, cooling the solution comprises cooling the solution by at least about 5° C., for example, at least about 10° C., or at least about 20° C. In some embodiments, cooling the solution comprises cooling the solution to about 15° C. to about 25° C., for example, to about 18° C. to about 22° C. For example, to about 20° C. In some embodiments, the solution is agitated (e.g., stirred) during and after cooling. In some embodiments, the agitation is performed for at least about 1 hour, e.g., at least about 6, 12, 18, 24, 36, 48, 60, or 72 hours. In some embodiments, the agitation is performed for about 1 hour to about 96 hours, about 1 hour to about 24 hours, about 24 hours to about 48 hours, about 48 hours to about 72 hours, about 72 hours to about 96 hours, about 48 hours to about 96 hours, about 54 hours to about 90 hours, about 60 hours to about 84 hours, about 66 hours to about 78 hours, about 70 hours to about 74 hours, or about 72 hours. In some embodiments, the solution becomes a suspension after cooling.

In some embodiments, filtering the suspension to provide the crystalline form comprises filtering the suspension to form a solid, washing the solid with a solvent, and drying the solid to provide the crystalline form. In some embodiments, the solvent comprises ethyl acetate, ethanol, diethyl ether, methyl tert-butyl ether, acetonitrile, tetrahydrofuran, isopropanol, methanol, or a combination thereof. In some embodiments, the solvent is ethyl acetate, ethanol, diethyl ether, methyl tert-butyl ether, acetonitrile, tetrahydrofuran, isopropanol, methanol, or a combination thereof. In some embodiments, the solvent comprises methanol, ethanol, and/or isopropanol. For example, the solvent comprises isopropanol. In some embodiments, the solvent is methanol, ethanol, or isopropanol. For example, the solvent is isopropanol.

In some embodiments, the crystalline form is Form A prepared by a method comprising:
(a) adding (S)-4-(2,2-difluoro-7-((5-methoxy-7-methyl-1H-indol-4-yl)methyl)-7-azaspiro[3.5]nonan-6-yl)benzoic acid to isopropanol at from about 40° C. to about 50° C. with stirring to form a solution of about 0.44 molar concentration;
(b) cooling the solution to about 20° C. and stirring for about 72 hours to form a suspension;
(c) filtering the suspension to obtain a solid;
(d) washing the solid with isopropanol; and
(e) drying the solid to provide the crystalline form.

### Methods of Preparing Form B

In some embodiments, the crystalline form is Form B prepared by a method comprising:
(a) adding an alcohol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension;
(b) heating the suspension to form a solution;
(c) cooling the solution, then adding ethyl acetate;
(d) optionally adding a suspension of crystalline (S)-4-(2,2-difluoro-7-((5-methoxy-7-methyl-1H-indol-4-yl)methyl)-7-azaspiro[3.5]nonan-6-yl)benzoic acid p-toluene sulfonic acid salt in a binary mixture of ethanol and ethyl acetate to form a mixture;
(e) adding ethyl acetate to form a slurry;
(f) filtering the slurry to form a solid;
(g) washing the solid with a solvent;
(h) drying the solid to provide the crystalline form;
(i) adding the solid form to a binary mixture of isopropanol and water then heating to form a slurry;
(j) cooling the slurry and filtering to form a solid; and
(k) drying the solid to provide the crystalline form.

In some embodiments, the (S)-4-(2,2-difluoro-7-((5-methoxy-7-methyl-1H-indol-4-yl)methyl)-7-azaspiro[3.5]nonan-6-yl)benzoic acid in step (a) is amorphous (S)-4-(2,2-difluoro-7-((5-methoxy-7-methyl-1H-indol-4-yl)methyl)-7-azaspiro[3.5]nonan-6-yl)benzoic acid.

In some embodiments, the alcohol comprises methanol, ethanol, and/or isopropanol. For example, the alcohol comprises ethanol. In some embodiments, the alcohol is methanol, ethanol, or isopropanol. For example, the alcohol is ethanol. In some embodiments, step (a) comprises adding about 1 to about 6 (e.g., about 2 to about 5, about 3 to about 4, or about 3.5) volumes of an alcohol relative to the (S)-4-(2,2-difluoro-7-((5-methoxy-7-methyl-1H-indol-4-yl)methyl)-7-azaspiro[3.5]nonan-6-yl)benzoic acid. In some embodiments, step (a) comprises adding about 3.5 volumes of an alcohol relative to the (S)-4-(2,2-difluoro-7-((5-methoxy-7-methyl-1H-indol-4-yl)methyl)-7-azaspiro[3.5]nonan-6-yl)benzoic acid.

In some embodiments, step (b) comprises heating the suspension to about 30° C. to about 70° C. (for example, about 40° C. to about 60° C., about 45° C., about 50° C., or about 55° C.). In some embodiments, step (b) comprises agitating the suspension. For example, step (b) comprises stirring the suspension. In some embodiments, step (b) comprises heating the suspension for about 1 minute to about 2 hours (e.g., about 5 minutes to about 1 hour, about 5 minutes to about 45 minutes, about 5 minutes to about 30 minutes, about 5 minutes to about 25 minutes, about 10 minutes to about 20 minutes, about 12 minutes to about 17 minutes, or about 15 minutes. For example, step (b) comprises heating the suspension for about 15 minutes.

In some embodiments, step (c) comprises cooling the solution to about 15° C. to about 50° C. (e.g., about 20° C. to about 30° C., about 22° C. to about 28° C., or about 25° C.). In some embodiments, step (c) comprises cooling the solution to about 25° C. In some embodiments, step (c) comprises agitating the solution. For example, step (c) comprises stirring the solution. In some embodiments, step (c) comprises cooling the solution for about 1 minute to about 2 hours (e.g., about 5 minutes to about 1 hour, about 5 minutes to about 45 minutes, about 5 minutes to about 30 minutes, about 5 minutes to about 25 minutes, about 10 minutes to about 20 minutes, about 12 minutes to about 17 minutes, or about 15 minutes. For example, step (c) comprises cooling the solution for about 15 minutes. In some embodiments, the ethyl acetate added to the solution is about 1 to about 4 (e.g., about 1 to about 3, about 2 to about 4, about 1, about 1.5, about 2, about 2.5, about 3, about 3.5, or about 4) volumes relative to the volume of the suspension. In some embodiments, the ethyl acetate added to the solution is about 2.5 volumes relative to the volume of the suspension.

In some embodiments, (d) is not performed. In some embodiments, (d) is performed. In some embodiments, the ratio of ethanol and ethyl acetate in the binary mixture in step (d) is in a ratio of about 3:1 to about 1:4 (e.g., about 2:1 to about 1:4, about 1:1 to about 1:4, about 1:2 to about 1:4, or about 1:3 by volume. For example, the ratio of ethanol and ethyl acetate in the binary mixture in step (d) is in a ratio of about 1:3 by volume. In some embodiments, addition of the binary mixture in step (d) is performed at about 15° C. to about 50° C. (e.g., about 20° C. to about 30° C., about 22° C. to about 28° C., or about 25° C.). In some embodiments, addition of the binary mixture in step (d) is performed at about 25° C.

In some embodiments, adding the ethyl acetate in step (d) is performed over about 15 minutes to about 4 hours (e.g., about 15 minutes to about 3 hours, about 1 hour to about 2.5 hours, about 1.5 hours to about 2.5 hours, about 1.75 hours to about 2.25 hours, or about 2 hours. For example, adding the ethyl acetate in step (d) is performed over about 2 hours. In some embodiments, adding the ethyl acetate in step (d) is performed at about 15° C. to about 50° C. (e.g., about 20° C. to about 30° C., about 22° C. to about 28° C., or about 25° C.). In some embodiments, adding the ethyl acetate in step (d) is performed at about 25° C. In some embodiments, about 1 to about 12 volumes (e.g., about 2 to about 10, about 4 to about 9, about 5 to about 9, about 7 to about 8, or about 7.5 volumes) of ethyl acetate is added relative to the volume of the mixture before addition of the ethyl acetate. In some embodiments, about 7.5 volumes of ethyl acetate is added relative to the volume of the (S)-4-(2,2-difluoro-7-((5-methoxy-7-methyl-1H-indol-4-yl)methyl)-7-azaspiro[3.5]nonan-6-yl)benzoic acid used in step (a)

In some embodiments, the solvent in step (g) comprises ethanol, ethyl acetate, or both. In some embodiments, the solvent in step (g) comprises ethanol. In some embodiments, the solvent in step (g) comprises ethyl acetate. In some embodiments, the solvent in step (g) is a binary mixture of ethanol and ethyl acetate. In some embodiments, the ratio of ethanol and ethyl acetate in the binary mixture is in a ratio of about 3:1 to about 1:4 (e.g., about 2:1 to about 1:4, about 1:1 to about 1:4, about 1:2 to about 1:4, or about 1:3 by volume. For example, the ratio of ethanol and ethyl acetate in the binary mixture is in a ratio of about 1:3 by volume. In some embodiments, the solid is washed with about 1 to about 4 volumes (e.g., about 1 to about 3, about 1.5 to about 2.5, or about 2 volumes) of the solvent relative to the volume of the (S)-4-(2,2-difluoro-7-((5-methoxy-7-methyl-1H-indol-4-yl)methyl)-7-azaspiro[3.5]nonan-6-yl)benzoic acid used in step (a) In some embodiments, the solid is washed with about 2 volumes of the solvent relative to the volume of the solid.

In some embodiments, the ratio of isopropanol and water in the binary mixture in step (i) is in an about 1:1 to about 20:1 (e.g., about 2:1 to about 20:1, about 5:1 to about 20:1, or about 9:1) ratio by volume. In some embodiments, the ratio of isopropanol and water in the binary mixture in step (i) is in an about 9:1 ratio by volume. In some embodiments, after adding the solid form to the binary mixture of isopropanol, heating is performed at about 30° C. to about 70° C. (for example, about 40° C. to about 60° C., about 45° C., about 50° C., or about 55° C.). In some embodiments, after adding the solid form to the binary mixture of isopropanol, heating is performed at about 50° C. In some embodiments, after adding the solid form to the binary mixture of isopropanol, heating is performed for about 1 hour to about 48 hours (e.g., about 5 hours to about 36 hours, about 12 hours to about 20 hours, about 14 hours to about 18 hours, about 15 hours to about 17 hours, or about 16 hours). In some embodiments, after adding the solid form to the binary mixture of isopropanol, heating is performed for about 16 hours.

In some embodiments, cooling the slurry in step (j) is performed at about 0° C. to about 35° C. (e.g., about 0° C. to about 30° C., about 5° C. to about 30° C., about 10° C. to about 30° C., about 15° C. to about 30° C., about 20° C. to about 30° C., about 22° C. to about 28° C., or about 25° C.). In some embodiments, cooling the slurry in step (j) is performed at about 25° C.

In some embodiments, drying the solid in step (k) is performed at about 30° C. to about 70° C. (e.g., about 35° C. to about 65° C., about 40° C. to about 60° C., about 45° C. to about 55° C., or about 50° C.). In some embodiments, drying the solid in step (k) is performed at about 50° C.

In some embodiments, the crystalline form is Form B prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution
(c) cooling the solution to about 25° C. over about 15 minutes, then adding about 2.5 volumes of ethyl acetate;
(d) adding about 7.5 volumes of ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume;
(g) drying the solid to provide a solid form;
(h) adding the solid form to a binary mixture of isopropanol and water in an about 9:1 ratio by volume then heating at about 50° C. for about 16 hours to form a slurry;
(i) cooling the slurry to about 25° C. and filtering to obtain a solid; and
(j) drying the solid at about 50° C. to provide the crystalline form.

In some embodiments, the crystalline form is Form B prepared by a method comprising:
(a) adding water and acetone to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension;
(b) heating the suspension to about 50° C. and stirring to form a solution followed by cooling the solution to room temperature;
(c) adding to the solution seed Form B crystals to form a suspension;
(d) adding to the suspension in step (c) about 2.2 volumes of water over several hours to form a suspension;
(e) cooling and maintaining the suspension in step (d) to below room temperature for several hours and filtering to obtain a solid; and
(f) drying the solid at about 40° C. to 50° C. to provide the crystalline form.

### Methods of Preparing Form C

In some embodiments, the crystalline form is Form C prepared by a method comprising:
(a) adding an alcohol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension;
(b) heating the suspension to form a solution;
(c) cooling the solution, then adding ethyl acetate;
(d) optionally adding a suspension of crystalline (S)-4-(2,2-difluoro-7-((5-methoxy-7-methyl-1H-indol-4-yl)methyl)-7-azaspiro[3.5]nonan-6-yl)benzoic acid p-toluene sulfonic acid salt in a binary mixture of ethanol and ethyl acetate to form a mixture;
(e) adding ethyl acetate to form a slurry;
(f) filtering the slurry to form a solid;
(g) washing the solid with a solvent; and
(h) drying the solid to provide the crystalline form.

In some embodiments, the (S)-4-(2,2-difluoro-7-((5-methoxy-7-methyl-1H-indol-4-yl)methyl)-7-azaspiro[3.5]nonan-6-yl)benzoic acid in step (a) is amorphous (S)-4-(2,2-difluoro-7-((5-methoxy-7-methyl-1H-indol-4-yl)methyl)-7-azaspiro[3.5]nonan-6-yl)benzoic acid.

In some embodiments, the alcohol comprises methanol, ethanol, and/or isopropanol. For example, the alcohol comprises ethanol. In some embodiments, the alcohol is methanol, ethanol, or isopropanol. For example, the alcohol is ethanol. In some embodiments, step (a) comprises adding about 1 to about 6 (e.g., about 2 to about 5, about 3 to about 4, or about 3.5) volumes of an alcohol relative to the (S)-4-(2,2-difluoro-7-((5-methoxy-7-methyl-1H-indol-4-yl)methyl)-7-azaspiro[3.5]nonan-6-yl)benzoic acid. In some embodiments, step (a) comprises adding about 3.5 volumes of an alcohol relative to the (S)-4-(2,2-difluoro-7-((5-methoxy-7-methyl-1H-indol-4-yl)methyl)-7-azaspiro[3.5]nonan-6-yl)benzoic acid.

In some embodiments, step (b) comprises heating the suspension to about 30° C. to about 70° C. (for example, about 40° C. to about 60° C., about 45° C., about 50° C., or about 55° C.). In some embodiments, step (b) comprises agitating the suspension. For example, step (b) comprises stirring the suspension. In some embodiments, step (b) comprises heating the suspension for about 1 minute to about 2 hours (e.g., about 5 minutes to about 1 hour, about 5 minutes to about 45 minutes, about 5 minutes to about 30 minutes, about 5 minutes to about 25 minutes, about 10 minutes to about 20 minutes, about 12 minutes to about 17 minutes, or about 15 minutes. For example, step (b) comprises heating the suspension for about 15 minutes.

In some embodiments, step (c) comprises cooling the solution to about 15° C. to about 50° C. (e.g., about 20° C. to about 30° C., about 22° C. to about 28° C., or about 25° C.). In some embodiments, step (c) comprises cooling the solution to about 25° C. In some embodiments, step (c) comprises agitating the suspension. For example, step (c) comprises stirring the solution. In some embodiments, step (c) comprises cooling the solution for about 1 minute to about 2 hours (e.g., about 5 minutes to about 1 hour, about 5 minutes to about 45 minutes, about 5 minutes to about 30 minutes, about 5 minutes to about 25 minutes, about 10 minutes to about 20 minutes, about 12 minutes to about 17 minutes, or about 15 minutes. For example, step (c) comprises cooling the solution for about 15 minutes. In some embodiments, the ethyl acetate added to the solution is about 1 to about 4 (e.g., about 1 to about 3, about 2 to about 4, about 1, about 1.5, about 2, about 2.5, about 3, about 3.5, or about 4) volumes relative to the volume of the solution. In some embodiments, the ethyl acetate added to the solution is about 2.5 volumes relative to the volume of the solution.

In some embodiments, (d) is not performed. In some embodiments, (d) is performed. In some embodiments, the ratio of ethanol and ethyl acetate in the binary mixture in step (d) is in a ratio of about 3:1 to about 1:4 (e.g., about 2:1 to about 1:4, about 1:1 to about 1:4, about 1:2 to about 1:4, or about 1:3 by volume. For example, the ratio of ethanol and ethyl acetate in the binary mixture in step (d) is in a ratio of about 1:3 by volume. In some embodiments, addition of the binary mixture in step (d) is performed at about 15° C. to about 50° C. (e.g., about 20° C. to about 30° C., about 22° C. to about 28° C., or about 25° C.). In some embodiments, addition of the binary mixture in step (d) is performed at about 25° C.

In some embodiments, adding the ethyl acetate in step (d) is performed over about 15 minutes to about 4 hours (e.g., about 15 minutes to about 3 hours, about 1 hour to about 2.5 hours, about 1.5 hours to about 2.5 hours, about 1.75 hours to about 2.25 hours, or about 2 hours. For example, adding the ethyl acetate in step (d) is performed over about 2 hours. In some embodiments, adding the ethyl acetate in step (d) is performed at about 15° C. to about 50° C. (e.g., about 20° C. to about 30° C., about 22° C. to about 28° C., or about 25° C.). In some embodiments, adding the ethyl acetate in step (e) is performed at about 25° C. In some embodiments, about 1 to about 12 volumes (e.g., about 2 to about 10, about 4 to about 9, about 5 to about 9, about 7 to about 8, or about 7.5 volumes) of ethyl acetate is added relative to the volume of the (S)-4-(2,2-difluoro-7-((5-methoxy-7-methyl-1H-indol-4-yl)methyl)-7-azaspiro[3.5]nonan-6-yl)benzoic acid used in step (a). In some embodiments, about 7.5 volumes of ethyl acetate is added relative to the volume of the mixture before addition of the ethyl acetate.

In some embodiments, the solvent in step (g) comprises ethanol, ethyl acetate, or both. In some embodiments, the solvent in step (g) comprises ethanol. In some embodiments, the solvent in step (g) comprises ethyl acetate. In some embodiments, the solvent in step (g) is a binary mixture of ethanol and ethyl acetate. In some embodiments, the ratio of ethanol and ethyl acetate in the binary mixture is in a ratio of about 3:1 to about 1:4 (e.g., about 2:1 to about 1:4, about 1:1 to about 1:4, about 1:2 to about 1:4, or about 1:3 by volume. For example, the ratio of ethanol and ethyl acetate in the binary mixture is in a ratio of about 1:3 by volume. In some embodiments, the solid is washed with about 1 to about 4 volumes (e.g., about 1 to about 3, about 1.5 to about 2.5, or about 2 volumes) of the solvent relative to the volume of the (S)-4-(2,2-difluoro-7-((5-methoxy-7-methyl-1H-indol-4-yl)methyl)-7-azaspiro[3.5]nonan-6-yl)benzoic acid used in step (a) In some embodiments, the solid is washed with about 2 volumes of the solvent relative to the volume of the solid.

In some embodiments, the crystalline form is Form C prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding about 2.5 volumes of ethyl acetate;
(d) adding ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume; and
(g) drying the solid to provide the crystalline form.

In some embodiments, the crystalline form is Form C prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding ethyl acetate;
(d) adding ethyl acetate over 2 hours at about 25° C. to form a slurry, then allowing the slurry to stand for about 1 hour at about 25° C.;
(e) filtering the slurry to form a solid;
(f) washing the solid with a binary mixture of ethanol and ethyl acetate in an about 1:3 ratio by volume; and
(g) drying the solid to provide the crystalline form.

### Methods of Preparing Form D

In some embodiments, the crystalline form is Form D prepared by a method comprising:
(a) preparing a solution of (S)-4-(2,2-difluoro-7-((5-methoxy-7-methyl-1H-indol-4-yl)methyl)-7-azaspiro[3.5]nonan-6-yl)benzoic acid in a solvent;
(b) adding a solution of hydrogen chloride in ethyl acetate or diethyl ether;
(c) adding a slurry of crystalline (S)-4-(2,2-difluoro-7-((5-methoxy-7-methyl-1H-indol-4-yl)methyl)-7-azaspiro[3.5]nonan-6-yl)benzoic acid hydrochloride in a solvent to form a suspension;
(d) adding about hydrogen chloride in ethyl acetate or diethyl ether to form a slurry;
(e) aging the slurry;
(f) filtering the slurry to form a solid;
(g) washing the solid with a solvent; and
(h) drying the solid to provide the crystalline form.

In some embodiments, the (S)-4-(2,2-difluoro-7-((5-methoxy-7-methyl-1H-indol-4-yl)methyl)-7-azaspiro[3.5]nonan-6-yl)benzoic acid in step (a) is amorphous (S)-4-(2,2-difluoro-7-((5-methoxy-7-methyl-1H-indol-4-yl)methyl)-7-azaspiro[3.5]nonan-6-yl)benzoic acid.

In some embodiments, the solvent in step (a) comprises ethyl acetate, ethanol, diethyl ether, methyl tert-butyl ether, acetonitrile, tetrahydrofuran, isopropanol, methanol, or a combination thereof. In some embodiments, the solvent in step (a) comprises ethyl acetate. In some embodiments, the solvent in step (a) is ethyl acetate. In some embodiments, the amount of solvent is about 2 to about 11 (e.g., about 3 to about 10, about 4 to about 7, about 5 to about 6, or about 5.5) volumes relative to the volume of the starting material. In some embodiments, the amount of solvent is about 5.5 volumes relative to the volume of the starting material.

In some embodiments, the solution of hydrogen chloride in ethyl acetate or diethyl ether is a solution of hydrogen chloride in ethyl acetate. In some embodiments, the concentration of the solution of hydrogen chloride is about 0.5 M to about 2 M (e.g., about 0.5 to about 1.5 M, about 0.7 M to about 2 M, about 0.8 M to about 1.2 M, or about 1 M). In some embodiments, the concentration of the solution of hydrogen chloride is about 1 M.

In some embodiments, the amount of hydrogen chloride added in step (b) relative to the (S)-4-(2,2-difluoro-7-((5-methoxy-7-methyl-1H-indol-4-yl)methyl)-7-azaspiro[3.5]nonan-6-yl)benzoic acid is about 0.1 to about 2.2 (e.g., about 0.2 to about 2, about 0.4 to about 1.2, about 0.4 to about 0.7, or about 0.55) equivalents. In some embodiments, the amount of hydrogen chloride added in step (b) relative to (S)-4-(2,2-difluoro-7-((5-methoxy-7-methyl-1H-indol-4-yl)methyl)-7-azaspiro[3.5]nonan-6-yl)benzoic acid is about 0.55 equivalents.

In some embodiments, the crystalline (S)-4-(2,2-difluoro-7-((5-methoxy-7-methyl-1H-indol-4-yl)methyl)-7-azaspiro[3.5]nonan-6-yl)benzoic acid hydrochloride used to form the suspension in step (c) is formed by combining a solution of (S)-4-(2,2-difluoro-7-((5-methoxy-7-methyl-1H-indol-4-yl)methyl)-7-azaspiro[3.5]nonan-6-yl)benzoic acid in ethyl acetate with a solution of 1 M hydrogen chloride in ethyl acetate to form a precipitate, then filtering the precipitate to provide the crystalline (S)-4-(2,2-difluoro-7-((5-methoxy-7-methyl-1H-indol-4-yl)methyl)-7-azaspiro[3.5]nonan-6-yl)benzoic acid hydrochloride. In some embodiments, the solution of (S)-4-(2,2-difluoro-7-((5-methoxy-7-methyl-1H-indol-4-yl)methyl)-7-azaspiro[3.5]nonan-6-yl)benzoic acid in ethyl acetate comprises less than 1% (e.g., less than 0.1%, less than 0.01%, less than 0.001%, less than 0.0001%, less than 0.00001%, or less than 0.000001%) water by weight. In some embodiments, the solution of 1 M hydrogen chloride in ethyl acetate comprises less than 1% (e.g., less than 0.1%, less than 0.01%, less than 0.001%, less than 0.0001%, less than 0.00001%, or less than 0.000001%) water by weight. In some embodiments, the solvent in step (c) comprises ethyl acetate, ethanol, diethyl ether, methyl tert-butyl ether, acetonitrile, tetrahydrofuran, isopropanol, methanol, or a combination thereof. In some embodiments, the solvent in step (c) comprises ethyl acetate. In some embodiments, the solvent in step (c) is ethyl acetate.

In some embodiments, the amount of hydrogen chloride added in step (d) relative to (S)-4-(2,2-difluoro-7-((5-methoxy-7-methyl-1H-indol-4-yl)methyl)-7-azaspiro[3.5]nonan-6-yl)benzoic acid is about 0.1 to about 2.2 (e.g., about 0.2 to about 2.2, about 0.8 to about 2, about 1 to about 2, about 1.3 to about 2, about 1.5 to about 1.8, or about 1.65) equivalents. In some embodiments, the amount of hydrogen chloride added in step (b) relative to (S)-4-(2,2-difluoro-7-((5-methoxy-7-methyl-1H-indol-4-yl)methyl)-7-azaspiro[3.5]nonan-6-yl)benzoic acid is about 1.65 equivalents.

In some embodiments, aging the slurry in step (e) comprises agitating the slurry. In some embodiments, agitating the slurry in step (e) comprises stirring the slurry. In some embodiments, after the slurry is aged for about 1 hour to about 48 hours (e.g., about 5 hours to about 36 hours, about 12 hours to about 20 hours, about 14 hours to about 18 hours, about 15 hours to about 17 hours, or about 16 hours). In some embodiments, the slurry is aged for about 16 hours. In some embodiments, the slurry is aged at about 0° C. to about 35° C. (e.g., about 0° C. to about 30° C., about 5° C. to about 30° C., about 10° C. to about 30° C., about 15° C. to about 30° C., about 20° C. to about 30° C., about 22° C. to about 28° C., or about 24° C.). In some embodiments, the slurry is aged for about 24° C.

In some embodiments, the solvent in step (g) comprises ethyl acetate, ethanol, diethyl ether, methyl tert-butyl ether, acetonitrile, tetrahydrofuran, isopropanol, methanol, or a combination thereof. In some embodiments, the solvent in step (g) comprises ethyl acetate. In some embodiments, the solvent in step (g) is ethyl acetate.

In some embodiments, the crystalline form is Form D prepared by a method comprising:
(a) preparing a solution of (S)-4-(2,2-difluoro-7-((5-methoxy-7-methyl-1H-indol-4-yl)methyl)-7-azaspiro[3.5]nonan-6-yl)benzoic acid in about 5.5 volumes of ethyl acetate;
(b) adding about 0.55 equivalents of a 1 molar concentration solution of hydrogen chloride in ethyl acetate;
(c) adding a slurry of crystalline (S)-4-(2,2-difluoro-7-((5-methoxy-7-methyl-1H-indol-4-yl)methyl)-7-azaspiro[3.5]nonan-6-yl)benzoic acid in ethyl acetate hydrochloride to form a suspension;
(d) adding about 1.65 equivalents of a 1 molar concentration solution of hydrogen chloride in ethyl acetate to form a slurry;
(e) stirring the slurry at about 24° C. for about 16 hours;
(f) filtering the slurry to obtain a solid;
(g) washing the solid with ethyl acetate; and
(h) drying the solid to provide the crystalline form.

### Formulations

In another aspect, provided herein is a pharmaceutical composition comprising a crystalline form as described herein. In some embodiments, the pharmaceutical composition comprises Form A and a pharmaceutically acceptable carrier. In some embodiments, the pharmaceutical composition comprises Form B and a pharmaceutically acceptable carrier. In some embodiments, the pharmaceutical composition comprises Form C and a pharmaceutically acceptable carrier. In some embodiments, the pharmaceutical composition comprises Form D and a pharmaceutically acceptable carrier. In some embodiments, the pharmaceutical composition comprises any combination of Form A, Form B, Form C, and Form D and a pharmaceutically acceptable carrier.

For example, the pharmaceutical composition comprises Form A and Form B and a pharmaceutically acceptable carrier.

For example, the pharmaceutical composition comprises Form A and Form C and a pharmaceutically acceptable carrier.

For example, the pharmaceutical composition comprises Form A and Form D and a pharmaceutically acceptable carrier.

For example, the pharmaceutical composition comprises Form B and Form C and a pharmaceutically acceptable carrier.

For example, the pharmaceutical composition comprises Form B and Form D and a pharmaceutically acceptable carrier.

For example, the pharmaceutical composition comprises Form C and Form D and a pharmaceutically acceptable carrier.

For example, the pharmaceutical composition comprises Form A, Form B, Form C, and a pharmaceutically acceptable carrier.

For example, the pharmaceutical composition comprises Form A, Form B, Form D, and a pharmaceutically acceptable carrier.

For example, the pharmaceutical composition comprises Form B, Form C, Form D, and a pharmaceutically acceptable carrier.

For example, the pharmaceutical composition comprises Form A, Form C, Form D, and a pharmaceutically acceptable carrier.

For example, the pharmaceutical composition comprises Form A, Form B, Form C, Form D, and a pharmaceutically acceptable carrier.

In some embodiments, the pharmaceutical composition includes at least about 0.5 weight percent (e.g., at least about 1 weight percent, at least about 2 weight percent, 5 weight percent, at least about 10 weight percent, at least about 20 weight percent, at least about 30 weight percent, at least about 40 weight percent, at least about 50 weight percent, at least about 60 weight percent, at least about 70 weight percent, at least about 80 weight percent, at least about 90 weight percent, at least about 95 weight percent, at least about 99 weight percent) of Form A, Form B, Form C, or Form D, or any combination thereof (e.g., any two, three, or four of crystalline Forms A, B, C, or D in combination.

Some embodiments provide a composition (e.g., a pharmaceutical composition or a pharmaceutical formulation) that includes one or more (e.g., 1 or 2, e.g., 1) active ingredients, in which the active ingredient (or at least one active ingredient):
(i) is Form A, Form B, Form C, or Form D, or any combination thereof (e.g., any two, three, or four of Forms A, B, C, or D in combination); or
(ii) includes at least about 0.5 weight percent (e.g., at least about 1 weight percent, at least about 2 weight percent, at least about 5 weight percent, at least about 10 weight percent, at least about 20 weight percent, at least about 30 weight percent, at least about 40 weight percent, at least about 50 weight percent, at least about 60 weight percent, at least about 70 weight percent, at least about 80 weight percent, at least about 90 weight percent, at least about 95 weight percent, at least about 99 weight percent) of one or more (e.g., 1 or 2, e.g., 1) of Form A, Form B, Form C, Form D, or any combination thereof (e.g., any two, three, or four of crystalline Forms A, B, C, or D in combination).

The compositions can include one or more of the following features.

The compositions can include one or more pharmaceutically acceptable carriers.

Solid dosage forms of the instant pharmaceutical compositions for oral administration include capsules, tablets, pills, powders, and granules. In one embodiment, the solid dosage form is a capsule. In one embodiment, the solid dosage form is a capsule filled with neat Form A, Form B, Form C, Form D, or any combination thereof. In some embodiments, the active compound is mixed with at least one inert pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the instant pharmaceutical compositions of tablets, dragées, capsules, pills, and granules can be prepared with coatings and shells such as enteric coatings and other pharmaceutical coatings. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Liquid dosage forms of the instant pharmaceutical compositions for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions of the instant compounds, in addition to the active compounds, may contain suspending agents as, for example, ethoxylated isostearyl alcohols, polyoxyethylene sorbitol and sorbitan esters (e.g., polyoxyethylene (20) sorbitan monooleate, i.e., polysorbate 80 or “Tween 80”), microcrystalline cellulose, aluminum metahydroxide, bentonite, agar-agar, and tragacanth, and mixtures thereof.

Pharmaceutical compositions of the present disclosure for injection comprise pharmaceutically acceptable sterile aqueous or non-aqueous solutions, dispersions, suspensions, or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants. Other excipients or carriers include, for example, kleptose (hydroxypropyl β-cyclodextrin) and water-soluble polymers derived from cellulose (e.g., methylcellulose (e.g., methocel) and hydroxypropyl methylcellulose).

In some embodiments, the pH of liquid (e.g., injectable) compositions is about 5 to about 12 (e.g., about 6 to about 11, about 7 to about 11, about 7 to about 10, about 7 to about 9, about 7.5 to about 8.0, about 8 to about 10.5, about 8.5 to about 10, about 8.5, about 9, about 9.5, or about 10). For example, the pH of liquid compositions is about 8.5 to about 10.

Besides inert diluents, these pharmaceutical compositions may also contain adjuvants such as preservative, wetting agents, emulsifying agents, dispersing agents, sweetening, flavoring, and perfuming agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin. The compounds can be incorporated into slow release or targeted delivery systems such as polymer matrices, liposomes, and microspheres. Such formulations may provide more effective distribution of the compounds.

The pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable medium prior to use.

Dosage forms for topical administration of a compound or pharmaceutical composition of the present disclosure include powders, patches, sprays, ointments, and inhalants. The active compound is mixed under sterile conditions with a pharmaceutically acceptable carrier and any preservatives, buffers, or propellants which may be required.

The compounds and compositions described herein can, for example, be administered orally, parenterally (e.g., subcutaneously, intracutaneously, intravenously or intramuscularly), topically, rectally, nasally sublingually or buccally, with a dosage ranging from about 0.01 milligrams per kilogram (mg/kg) to about 1000 mg/kg, (e.g., from about 0.01 to about 100 mg/kg, from about 0.1 to about 100 mg/kg) every 4 to 120 hours, or according to the requirements of the particular drug, dosage form, and/or route of administration. Other routes of administration include enteric, intraarterial, intraperitoneal and intrathecal administration. The interrelationship of dosages for animals and humans (based on milligrams per meter squared of body surface) is described by Freireich et al., Cancer Chemother. Rep. 50, 219-244 (1966). Body surface area may be approximately determined from height and weight of the patient. See, e.g., Scientific Tables, Geigy Pharmaceuticals, Ardsley, N.Y., 537 (1970).

In some embodiments, the composition comprises Form A, Form B, Form C, or Form D; hydroxypropyl β-cyclodextrin; and water. In some embodiments, the composition comprises Form A; hydroxypropyl β-cyclodextrin; and water. In some embodiments, the composition comprises Form B; hydroxypropyl β-cyclodextrin; and water. In some embodiments, the composition comprises Form C; hydroxypropyl β-cyclodextrin; and water. In some embodiments, the composition comprises Form D; hydroxypropyl β-cyclodextrin; and water.

In some embodiments, the composition comprises Form A, Form B, Form C, or Form D; methocel; tween 80; and water. In some embodiments, the composition comprises Form A; methocel; tween 80; and water. In some embodiments, the composition comprises Form B; methocel; tween 80; and water. In some embodiments, the composition comprises Form C; methocel; tween 80; and water. In some embodiments, the composition comprises Form D; methocel; tween 80; and water.

In some embodiments, the crystalline form (e.g., Form A, Form B, Form C, or Form D) has a particle size (D₅₀) of about 1 μm to about 100 μm, for example, about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm. In some embodiments, the crystalline form (e.g., Form A, Form B, Form C, or Form D) has a particle size (D₅₀) of about 1 μm to about 50 μm. In some embodiments, the crystalline form (e.g., Form A, Form B, Form C, or Form D) has a particle size (D₅₀) of about 1 μm to about 25 μm. In some embodiments, the crystalline form (e.g., Form A, Form B, Form C, or Form D) has a particle size (D₅₀) of about 1 μm to about 5 μm.

### Methods of Use

Provided herein are methods of using the crystalline forms disclosed herein, or pharmaceutical compositions thereof, for the treatment, prevention or amelioration of a disease or disorder that is mediated by or otherwise affected via complement alternative pathway. In yet certain embodiments, provided herein are methods of using the crystalline forms disclosed herein, or pharmaceutical compositions thereof, for the treatment, prevention or amelioration of a disease or disorder that is mediated by or otherwise affected via complement factor B (CFB). In yet certain embodiments, provided herein are methods of using the crystalline forms disclosed herein, or pharmaceutical compositions thereof, for the treatment, prevention or amelioration of a disease or disorder that is mediated by or otherwise affected via inhibition of the complement alternative pathway. In yet certain embodiments, provided herein are methods of using the crystalline forms disclosed herein, or pharmaceutical compositions thereof, for the treatment, prevention or amelioration of a disease or disorder that is mediated by or otherwise affected via inhibition of complement factor B.

In some embodiments, the crystalline form is Form A. In some embodiments, the crystalline form is Form B. In some embodiments, the crystalline form is Form C. In some embodiments, the crystalline form is Form D.

Some embodiments provide a method of treating or preventing a disease or disorder as described herein (e.g., a complement related disease or disorder) in a subject in need thereof, comprising administering to the subject a therapeutically effective amount of Form A, Form B, Form C, or Form D, or a pharmaceutical composition comprising Form A, Form B, Form C, or Form D, and a pharmaceutically acceptable carrier.

Examples of known complement related diseases or disorders include: neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during I L-2 therapy, inflammatory disorders, inflammation of autoimmune diseases, Crohn's disease, adult respiratory distress syndrome, thermal injury including burns or frostbite, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration and neural regeneration. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, Pauci-immune vasculitis, immune complex-associated inflammation; eye diseases including age related macular degeneration, diabetic retinopathy, retinitis pigmentosa, macular edema, Behcet's uveitis, multifocal choroiditis, Vogt-Koyangi-Harada syndrome, intermediate uveitis, birdshot retino-choroiditis, sympathetic ophthalmia, ocular cicatricial pemphigoid, ocular pemphigus, nonarteritic ischemic optic neuropathy, post-operative inflammation, and retinal vein occlusion uveitis (including Behcet's disease and other sub-types of uveitis), antiphospholipid syndrome.

In some embodiments, the disease or disorder is selected from the group consisting of: age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North Carolina macular dystrophy, Stargardt disease, corneal inflammation, neurological disorders such as multiple sclerosis, stroke, Guillain Barré Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity, disorders of inappropriate or undesirable complement activation such as hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), inflammation or autoimmune diseases such as Crohn's disease, neuromyelitis optica (NMO), IgA vasculitis (formerly known as Henoch-Schönlein purpura or HSP), hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, renal ischemia, acute kidney injury mesenteric artery reperfusion after aortic reconstruction, infectious disease or sepsis; COVID-19, immune complex disorders and autoimmune diseases, rheumatoid osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity; immune thrombocytopenia, Cold agglutinin disease, Warm autoimmune hemolytic anemia (wAIHA), thrombotic thrombocytopenia purpura (TTP), abdominal aortic aneurisms, Grave's disease, and hidradenitis suppurativa.

In some embodiments, the disease or disorder is selected from the group consisting of: multiple sclerosis, Guillain Barre Syndrome, traumatic brain injury, Parkinson's disease, age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-choroiditis, sympathetic ophthalmia, ocular cicatricial pemphigoid, ocular pemphigus, nonarteritic ischemic optic neuropathy, post-operative inflammation, retinal vein occlusion, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, stroke, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, metabolic syndrome, and hidradenitis suppurativa.

In some embodiments, the disease or disorder is immune complex membranoproliferative glomerulonephritis (IC-MPGN).

In some embodiments, the disease or disorder is neuromyelitis optica (NMO).

In some embodiments, the disease or disorder is hematopoietic stem cell transplantation-associated thrombotic microangiopathy (HSCT-TMA).

Some embodiments provide a method of treating or preventing a kidney disease or disorder selected from the group consisting of: chronic kidney disease, diabetic nephropathy, glomerular kidney disease, complement C3 glomerulopathy (C3G), IgA nephropathy (IgAN), membranous nephropathy (MN), focal segmental glomerulosclerosis (FSGS), atypical hemolytic uremic syndrome (aHUS), dense-deposit disease (DDD), minimal change disease (MCD), paroxysmal nocturnal hemoglobinuria (PNH), ANCA-associated vasculitis, lupus nephritis and polycystic kidney disease (PKD), comprising administering to a subject having such disease or disorder, a therapeutically effective amount of Form A, Form B, Form C, or Form D, or a pharmaceutical composition comprising Form A, Form B, Form C, or Form D, and a pharmaceutically acceptable carrier.

In some embodiments, the kidney disease is selected from the group consisting of: chronic kidney disease, diabetic nephropathy, glomerular kidney disease, complement C3 glomerulopathy (C3G), IgA nephropathy (IgAN), membranous nephropathy (MN), focal segmental glomerulosclerosis (FSGS), atypical hemolytic uremic syndrome (aHUS), dense-deposit disease (DDD), minimal change disease (MCD), paroxysmal nocturnal hemoglobinuria (PNH), ANCA-associated vasculitis, lupus nephritis, polycystic kidney disease (PKD), and immune complex membranoproliferative glomerulonephritis (IC-MPGN).

In some embodiments, the kidney disease is chronic kidney disease.

In some embodiments, the kidney disease is diabetic nephropathy.

In some embodiments, the kidney disease is glomerular kidney disease.

In some embodiments, the kidney disease is complement C3 glomerulopathy (C3G).

In some embodiments, the kidney disease is IgA nephropathy (IgAN).

In some embodiments, the kidney disease is membranous nephropathy (MN).

In some embodiments, the kidney disease is focal segmental glomerulosclerosis (FSGS).

In some embodiments, the kidney disease is atypical hemolytic uremic syndrome (aHUS).

In some embodiments, the kidney disease is dense-deposit disease (DDD).

In some embodiments, the kidney disease is minimal change disease (MCD).

In some embodiments, the kidney disease is paroxysmal nocturnal hemoglobinuria (PNH).

In some embodiments, the kidney disease is ANCA-associated vasculitis.

In some embodiments, the kidney disease is lupus nephritis.

In some embodiments, the kidney disease is polycystic kidney disease (PKD).

In some embodiments, the kidney disease is immune complex membranoproliferative glomerulonephritis (IC-MPGN).

The disclosure will be further described in the following examples. It should be understood that these examples are for illustrative purposes only and are not to be construed as limiting this disclosure in any manner.

### Additional Embodiments

1. A crystalline form of (S)-4-(2,2-difluoro-7-((5-methoxy-7-methyl-1H-indol-4-yl)methyl)-7-azaspiro[3.5]nonan-6-yl)benzoic acid.

2. The crystalline form of embodiment 1, wherein the crystalline form is Form A characterized by an X-ray powder diffraction (XRPD) pattern having a peak at 10.7±0.2 degrees 2θ.

3. The crystalline form of embodiment 2, wherein the XRPD pattern has a peak at 20.5±0.2 degrees 2θ.

4. The crystalline form of any one of embodiments 2-3, wherein the XRPD pattern has a peak at 18.8±0.2 degrees 2θ.

5. The crystalline form of any one of embodiments 2-4, wherein the XRPD pattern has a peak at 21.7±0.2 degrees 2θ.

6. The crystalline form of any one of embodiments 2-5, wherein the XRPD pattern has a peak at 19.6±0.2 degrees 2θ.

7. The crystalline form of any one of embodiments 2-6, wherein the XRPD pattern has a peak at 19.8±0.2 degrees 2θ.

8. The crystalline form of any one of embodiments 2-7, wherein the XRPD pattern has a peak at 12.5±0.2 degrees 2θ.

9. The crystalline form of any one of embodiments 2-8, wherein the XRPD pattern has a peak at 21.1±0.2 degrees 2θ.

10. The crystalline form of any one of embodiments 2-9, wherein the XRPD pattern has a peak at 23.3±0.2 degrees 2θ.

11. The crystalline form of any one of embodiments 2-10, wherein the XRPD pattern has a peak at 22.6±0.2 degrees 2θ.

12. The crystalline form of any one of embodiments 2-11, wherein the XRPD pattern has a peak at 27.3±0.2 degrees 2θ.

13. The crystalline form of any one of embodiments 2-12, wherein the XRPD pattern has a peak at 15.6±0.2 degrees 2θ.

14. The crystalline form of embodiment 1, wherein the crystalline form is Form A, and wherein the XRPD pattern has peaks (±0.2 degrees 2θ) at 10.7, 20.5, and 18.8.

15. The crystalline form of embodiment 1, wherein the crystalline form is Form A, and wherein the XRPD pattern has peaks (±0.2 degrees 2θ) at 10.7, 20.5, 18.8, and 21.7.

16. The crystalline form of embodiment 1, wherein the crystalline form is Form A, and wherein the XRPD pattern has peaks (±0.2 degrees 2θ) at 10.7, 20.5, 18.8, 21.7, 19.6, 19.8, 12.5, 21.1, 23.3, 22.5, 27.3, and 15.5.

17. The crystalline form of embodiment 1, wherein the crystalline form is Form A, the (S)-4-(2,2-difluoro-7-((5-methoxy-7-methyl-1H-indol-4-yl)methyl)-7-azaspiro[3.5]nonan-6-yl)benzoic acid is in a free form, further wherein the free form is an anhydrate form.

18. The crystalline form of embodiment 1, wherein the crystalline form is Form A, and wherein the XRPD pattern is substantially the same as that shown in FIG. 2.

19. The crystalline form of any one of embodiments 1-18, wherein the crystalline form is Form A having a thermogravimetric analysis (TGA) curve characterized by a weight loss of about 2% at about 189° C.

20. The crystalline form of any one of embodiments 1-19, wherein the crystalline form is Form A having a TGA curve characterized by a weight loss of about 25% at about 300° C.

21. The crystalline form of any one of embodiments 1-20, wherein the crystalline form is Form A having a TGA curve that is substantially the same as that shown in FIG. 3.

22. The crystalline form of any one of embodiments 1-21, wherein the crystalline form is Form A having a differential scanning calorimetry (DSC) curve characterized by a melting onset of about 201.9° C. (endo).

23. The crystalline form of any one of embodiments 1-22, wherein the crystalline form is Form A having a DSC curve that is substantially the same as that shown in FIG. 4.

24. The crystalline form of any one of embodiments 1-23, wherein the crystalline form is Form A characterized by having a solubility of about 2.3 mg/mL in Fasted State Simulated Gastric Fluid (FaSSGF) at about 37° C. after about 30 minutes.

25. The crystalline form of any one of embodiments 1-24, wherein the crystalline form is Form A characterized by having a solubility of about 0.31 mg/mL in Fed State Simulated Intestinal Fluid (FeSSIF) at about 37° C. after about 24 hours.

26. The crystalline form of any one of embodiments 1-25, wherein the crystalline form is Form A characterized by having a solubility of about 0.18 mg/mL in Fasted State Simulated Intestinal Fluid (FaSSIF) at about 37° C. after about 24 hours.

27. The crystalline form of any one of embodiments 1-26, wherein the crystalline form is Form A characterized by having a solubility of about 0.22 mg/ml in water at about 37° C. after about 24 hours.

28. The crystalline form of any one of embodiments 1-27, wherein the crystalline form is Form A prepared by a method comprising:
(a) adding (S)-4-(2,2-difluoro-7-((5-methoxy-7-methyl-1H-indol-4-yl)methyl)-7-azaspiro[3.5]nonan-6-yl)benzoic acid to isopropanol from about 40 to about 50° C. with stirring to form a solution of about 0.44 molar concentration;
(b) cooling the solution to about 20° C. and stirring for about 72 hours to form a suspension;
(c) filtering the suspension to obtain a solid;
(d) washing the solid with isopropanol; and
(e) drying the solid to provide the crystalline form.

29. A crystalline p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid.

30. The crystalline form of embodiment 29, wherein the crystalline form is Form B characterized by an XRPD pattern having a peak at 11.8±0.2 degrees 2θ.

31. The crystalline form of embodiment 30, wherein the XRPD pattern has a peak at 9.3±0.2 degrees 2θ.

32. The crystalline form of any one of embodiments 29-31, wherein the XRPD pattern has a peak at 19.9±0.2 degrees 2θ.

33. The crystalline form of any one of embodiments 29-32, wherein the XRPD pattern has a peak at 22.9±0.2 degrees 2θ.

34. The crystalline form of any one of embodiments 29-33, wherein the XRPD pattern has a peak at 17.2±0.2 degrees 2θ.

35. The crystalline form of any one of embodiments 29-34, wherein the XRPD pattern has a peak at 10.2±0.2 degrees 2θ.

36. The crystalline form of any one of embodiments 29-35, wherein the XRPD pattern has a peak at 20.4±0.2 degrees 2θ.

37. The crystalline form of any one of embodiments 29-36, wherein the XRPD pattern has a peak at 21.3±0.2 degrees 2θ.

38. The crystalline form of any one of embodiments 29-37, wherein the XRPD pattern has a peak at 14.2±0.2 degrees 2θ. 39. The crystalline form of embodiment 29, wherein the crystalline form is Form B, and wherein the XRPD pattern has peaks (±0.2 degrees 2θ) at 11.80, 9.30, and 19.9.

40. The crystalline form of embodiment 29, wherein the crystalline form is Form B, and wherein the XRPD pattern has peaks (±0.2 degrees 2θ) at 11.8, 9.3, 19.9, and 22.9.

41. The crystalline form of embodiment 29, wherein the crystalline form is Form B, and wherein the XRPD pattern has peaks (±0.2 degrees 2θ) at 11.8, 9.3, 19.9, 22.9, 17.2, 10.2, 20.4, 21.3, and 14.2.

42. The crystalline form of embodiment 29, wherein the crystalline form is Form B, and wherein the XRPD pattern is substantially the same as that shown in FIG. 12 or 15.

43. The crystalline form of any one of embodiments 29-42, wherein the crystalline form is Form B having a TGA curve characterized by a weight loss of about 1% loss over about 60° C. to about 100° C.

44. The crystalline form of any one of embodiments 29-43, wherein the crystalline form is Form B having a TGA curve that is substantially the same as that shown in FIG. 13 or 17.

45. The crystalline form of any one of embodiments 29-44, wherein the crystalline form is Form B having a DSC curve that is substantially the same as that shown in FIG. 14 or 16.

46. The crystalline form of any one of embodiments 29-45, wherein the crystalline form is Form B characterized by having a solubility of about 0.54 mg/mL in Fasted State Simulated Gastric Fluid (FaSSGF) at about 37° C. after about 24 hours.

47. The crystalline form of any one of embodiments 29-46, wherein the crystalline form is Form B characterized by having a solubility of greater than about 2 mg/mL in Fed State Simulated Intestinal Fluid (FeSSIF) at about 37° C. after about 24 hours.

48. The crystalline form of any one of embodiments 29-47, wherein the crystalline form is Form B characterized by having a solubility of about 0.53 mg/mL in Fasted State Simulated Intestinal Fluid (FaSSIF) at about 37° C. after about 24 hours.

49. The crystalline form of any one of embodiments 29-48, wherein the crystalline form is Form B characterized by having a solubility of about 0.56 mg/ml in water at about 37° C. after about 24 hours.

50. The crystalline form of any one of embodiments 29-49, wherein the crystalline form is Form B prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding about 2.5 volumes of ethyl acetate;
(d) adding about 7.5 volumes of ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume;
(g) drying the solid to provide a solid form;
(h) adding the solid form to a binary mixture of isopropanol and water in an about 9:1 ratio by volume then heating at about 50° C. for about 16 hours to form a slurry;
(i) cooling the slurry to about 25° C. and filtering to obtain a solid; and
(j) drying the solid at about 50° C. to provide the crystalline form.

51. The crystalline form of embodiment 29, wherein the crystalline form is Form C characterized by an XRPD pattern having a peak at 22.3±0.2 degrees 2θ.

52. The crystalline form of embodiment 51, wherein the XRPD pattern has a peak at 17.3±0.2 degrees 2θ.

53. The crystalline form of any one of embodiments 51-52, wherein the XRPD pattern has a peak at 17.5±0.2 degrees 2θ.

54. The crystalline form of any one of embodiments 51-53, wherein the XRPD pattern has a peak at 21.8±0.2 degrees 2θ.

55. The crystalline form of any one of embodiments 51-54, wherein the XRPD pattern has a peak at 11.5±0.2 degrees 2θ.

56. The crystalline form of any one of embodiments 51-55, wherein the XRPD pattern has a peak at 15.3±0.2 degrees 2θ.

57. The crystalline form of any one of embodiments 51-56, wherein the XRPD pattern has a peak at 10.2±0.2 degrees 2θ.

58. The crystalline form of any one of embodiments 51-57, wherein the XRPD pattern has a peak at 10.7±0.2 degrees 2θ.

59. The crystalline form of any one of embodiments 51-58, wherein the XRPD pattern has a peak at 26.6±0.2 degrees 2θ.

60. The crystalline form of any one of embodiments 29-59, wherein Form B includes water, wherein water is in an amount from 0% to 2.7% w/w relative to a total weight of Form B.

61. The crystalline form of embodiment 29, wherein the crystalline form is Form C, and wherein the XRPD pattern has peaks (±0.2 degrees 2θ) at 22.3, 17.3, and 17.5.

62. The crystalline form of embodiment 29, wherein the crystalline form is Form C, and wherein the XRPD pattern has peaks (±0.2 degrees 2θ) at 22.3, 17.3, 17.5, and 21.8.

63. The crystalline form of embodiment 29, wherein the crystalline form is Form C, and wherein the XRPD pattern has peaks (±0.2 degrees 2θ) at 22.3, 17.3, 17.5, 21.8, 11.5, 15.3, 10.2, 10.7, and 26.6.

64. The crystalline form of embodiment 29, wherein the crystalline form is Form C, and wherein the XRPD pattern is substantially the same as that shown in FIG. 23.

65. The crystalline form of embodiment 29, wherein the p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid is in an anhydrate form.

66. The crystalline form of any one of embodiments 29 and 61-65, wherein the crystalline form is Form C having a TGA curve characterized by a weight loss of about 0.4% from about 210 to about 240° C.

67. The crystalline form of any one of embodiments 29 and 61-66, wherein the crystalline form is Form C having a TGA curve that is substantially the same as that shown in FIG. 24.

68. The crystalline form of any one of embodiments 29 and 61-67, wherein the crystalline form is Form C having a DSC curve characterized by a melting onset of about 187° C.

69. The crystalline form of any one of embodiments 29 and 61-68, wherein the crystalline form is Form C having a DSC curve that is substantially the same as that shown in FIG. 25.

70. The crystalline form of any one of embodiments 29 and 61-69, wherein the crystalline form is Form C characterized by having a solubility of about 0.85 mg/mL in Fasted State Simulated Gastric Fluid (FaSSGF) at about 37° C. after about 24 hours.

71. The crystalline form of any one of embodiments 29 and 61-70, wherein the crystalline form is Form C characterized by having a solubility of about 1.69 mg/mL in Fed State Simulated Intestinal Fluid (FeSSIF) at about 37° C. after about 24 hours.

72. The crystalline form of any one of embodiments 29 and 61-71, wherein the crystalline form is Form C characterized by having a solubility of about 0.13 mg/mL in Fasted State Simulated Intestinal Fluid (FaSSIF) at about 37° C. after about 24 hours.

73. The crystalline form of any one of embodiments 29 and 61-72, wherein the crystalline form is Form C characterized by having a solubility of about 1.12 mg/mL in water at about 37° C. after about 24 hours.

74. The crystalline form of any one of embodiments 29 and 61-73, wherein the crystalline form is Form C prepared by a method comprising:
(a) adding ethanol to (S)-4-(2,2-difluoro-7-((5-methoxy-7-methyl-1H-indol-4-yl)methyl)-7-azaspiro[3.5]nonan-6-yl)benzoic acid and p-toluenesulfonic acid to form a suspension, wherein the ethanol is about 3.5 volumes relative to the (S)-4-(2,2-difluoro-7-((5-methoxy-7-methyl-1H-indol-4-yl)methyl)-7-azaspiro[3.5]nonan-6-yl)benzoic acid;
(b) heating the suspension to about 50° C. and stirring for about 15 minutes to form a solution;
(c) cooling the solution to about 25° C. over about 15 minutes, then adding about 2.5 volumes of ethyl acetate;
(d) adding about 7.5 volumes of ethyl acetate over 2 hours at about 25° C. to form a slurry, then allowing the slurry to stand for about 1 hour at about 25° C.;
(e) filtering the slurry to obtain a solid;
(f) washing the solid with a binary mixture of ethanol and ethyl acetate in an about 1:3 ratio by volume; and
(g) drying the solid to provide the crystalline form.

75. A crystalline hydrochloride salt form of (S)-4-(2,2-difluoro-7-((5-methoxy-7-methyl-1H-indol-4-yl)methyl)-7-azaspiro[3.5]nonan-6-yl)benzoic acid.

76. The crystalline form of embodiment 75, wherein the crystalline form is Form D characterized by an XRPD pattern comprising a peak at 13.1±0.2 degrees 2θ.

77. The crystalline form of embodiment 75, wherein the XRPD pattern has a peak at 16.4±0.2 degrees 2θ.

78. The crystalline form of any one of embodiments 75-77, wherein the XRPD pattern has a peak at 10.4±0.2 degrees 2θ.

79. The crystalline form of any one of embodiments 75-78, wherein the XRPD pattern has a peak at 16.6±0.2 degrees 2θ.

80. The crystalline form of any one of embodiments 75-79, wherein the XRPD pattern has a peak at 23.4±0.2 degrees 2θ.

81. The crystalline form of any one of embodiments 75-80, wherein the XRPD pattern has a peak at 18.2±0.2 degrees 2θ.

82. The crystalline form of any one of embodiments 75-81, wherein the XRPD pattern has a peak at 15.9±0.2 degrees 2θ.

83. The crystalline form of any one of embodiments 75-82, wherein the XRPD pattern has a peak at 24.9±0.2 degrees 2θ.

84. The crystalline form of any one of embodiments 75-83, wherein the XRPD pattern has a peak at 17.5±0.2 degrees 2θ.

85. The crystalline form of embodiment 75, wherein the crystalline form is Form D, and wherein the XRPD pattern has peaks (±0.2 degrees 2θ) at 13.1, 16.4, and 10.4.

86. The crystalline form of embodiment 75, wherein the crystalline form is Form D, and wherein the XRPD pattern has peaks (±0.2 degrees 2θ) at 13.1, 16.4, 10.4, 16.6, and 23.4.

87. The crystalline form of embodiment 75, wherein the crystalline form is Form D, and wherein the XRPD pattern has peaks (±0.2 degrees 2θ) at 13.1, 16.4, 10.4, 16.6, 23.4, 18.2, 15.9, 24.9, and 17.5.

88. The crystalline form of embodiment 75, wherein the crystalline form is Form D, and wherein the XRPD pattern is substantially the same as that shown in FIG. 26.

89. The crystalline form of any one of embodiments 75-88, wherein the crystalline form is Form D characterized by a TGA curve indicating a weight loss of about 0.7% between about 210 to about 230° C.

90. The crystalline form of any one of embodiments 75-89, wherein the crystalline form is Form D characterized by a TGA curve that is substantially the same as that shown in FIG. 27.

91. The crystalline form of any one of embodiments 75-90, wherein the crystalline form is Form D characterized by a DSC curve that is substantially the same as that shown in FIG. 28.

92. The crystalline form of any one of embodiments 75-91, wherein the crystalline form is Form D characterized by having a solubility of about 2.25 mg/mL in Fasted State Simulated Gastric Fluid (FaSSGF) at about 37° C. after about 30 minutes.

93. The crystalline form of any one of embodiments 75-92, wherein the crystalline form is Form D characterized by having a solubility of about 2.33 mg/ml in Fed State Simulated Intestinal Fluid (FeSSIF) at about 37° C. after about 24 hours.

94. The crystalline form of any one of embodiments 75-93, wherein the crystalline form is Form D characterized by having a solubility of about 2.19 mg/mL in Fasted State Simulated Intestinal Fluid (FaSSIF) at about 37° C. after about 30 minutes.

95. The crystalline form of any one of embodiments 75-94, wherein the crystalline form is Form D characterized by having a solubility of about 2.08 mg/mL in water at about 37° C. after about 30 minutes.

96. The crystalline form of any one of embodiments 75-95, wherein the crystalline form is Form D prepared by a method comprising:
(a) preparing a solution of (S)-4-(2,2-difluoro-7-((5-methoxy-7-methyl-1H-indol-4-yl)methyl)-7-azaspiro[3.5]nonan-6-yl)benzoic acid in about 5.5 volumes of ethyl acetate;
(b) adding about 0.55 equivalents of a 1 molar concentration solution of hydrogen chloride in ethyl acetate;
(c) adding a slurry of crystalline (S)-4-(2,2-difluoro-7-((5-methoxy-7-methyl-1H-indol-4-yl)methyl)-7-azaspiro[3.5]nonan-6-yl)benzoic acid hydrochloride in ethyl acetate to form a suspension;
(d) adding about 1.65 equivalents of a 1 molar concentration solution of hydrogen chloride in ethyl acetate to form a slurry;
(e) stirring the slurry at about 24° C. for about 16 hours;
(f) filtering the slurry to obtain a solid;
(g) washing the solid with ethyl acetate; and
(h) drying the solid to provide the crystalline form.

97. A pharmaceutical composition comprising the crystalline form of any one of embodiments 1-96 and a pharmaceutically acceptable carrier.

98. A method of treating a disease or disorder associated with complement factor B (CFB), comprising administering to a subject having such disease or disorder, a therapeutically effective amount of the crystalline form of any one of embodiments 1-96, or the pharmaceutical composition of embodiment 94.

99. A method of treating or preventing a disease or disorder selected from autoimmune disease or disorder, inflammatory disease or disorder, metabolic disease or disorder, neurological disease or disorder, pulmonary disease, respiratory disease or disorder, ophthalmic disease, cardiovascular disease, and kidney disease, comprising administering to a subject having such disease or disorder, a therapeutically effective amount of the crystalline form of any one of embodiments 1-96, or the pharmaceutical composition of embodiment 97.

100. A method of treating or preventing a disease or disorder selected from multiple sclerosis, Guillain Barre Syndrome, traumatic brain injury, Parkinson's disease, age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-choroiditis, sympathetic ophthalmia, ocular cicatricial pemphigoid, ocular pemphigus, nonarteritic ischemic optic neuropathy, post-operative inflammation, retinal vein occlusion, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, stroke, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis, obesity, metabolic syndrome, and hidradenitis suppurativa, comprising administering to a subject having such disease or disorder, a therapeutically effective amount of the crystalline form of any one of embodiments 1-96, or the pharmaceutical composition of embodiment 97.

101. A method of treating or preventing a disease or disorder selected from kidney disease, chronic kidney disease, diabetic nephropathy, glomerular kidney disease, complement C3 glomerulopathy (C3G), IgA nephropathy (IgAN), membranous nephropathy (MN), focal segmental glomerulosclerosis (FSGS), atypical hemolytic uremic syndrome (aHUS), dense-deposit disease (DDD), minimal change disease (MCD), paroxysmal nocturnal hemoglobinuria (PNH), ANCA-associated vasculitis, lupus nephritis and polycystic kidney disease (PKD), comprising administering to a subject having such disease or disorder, a therapeutically effective amount of the crystalline form of any one of embodiments 1-96, or the pharmaceutical composition of embodiment 97.

102. A crystalline form according to any one of embodiments 1-96, or a pharmaceutical composition of embodiment 97, for use in the treatment of a disease or disorder of embodiments 99 or 100 in a subject in need of such treatment.

### EXAMPLES

### Materials and Methods

### X-Ray Powder Diffraction (XRPD)

XRPD Analysis was conducted on a Bruker D8-Advance X-ray diffractometer under Bragg-Brentano configuration using Copper Kα₁ & Kα₂ radiation at 1.5418 Å, a nickel filter and a LynxEye silicon strip detector (40 kV, 40 mA). The slit was set at 0.6 mm divergent, 8 mm anti-scatter, 2.5° soller, and XRPD was obtained from 1 to 60 2θ degrees.

### Thermogravimetric Analysis (TGA) and Differential Scanning Calorimetry (DSC)

TGA and DSC were performed on the same sample simultaneously using a Mettler Toledo TGA/DSC³⁺. Protective and purge gas was nitrogen at a flow rate of 20-30 mL/min and 50-100 mL/min, respectively. The desired amount of sample (5-10 mg) was weighed directly in a hermetic aluminum pan with pinhole and analyzed according to the parameters below:

Parameters

| Method | Ramp | |
|---|---|---|
| Sample size | 5-10 | mg |
| Heating rate | 10.0° | C./min |
| Temperature range | 30 to 300° | C. |

### Alternative TGA and DSC Methods

TGA method

| Instrument | TA Discovery | |
|---|---|---|
| Temperature range | Room temperature to 300° C. | |
| Scan rate | 10 | K/min |
| Nitrogen flow | 20 | mL/min |

| | |
|---|---|
| Sample mass | Approximately 2-10 mg |

DSC method

| Instrument | TA Discovery | |
|---|---|---|
| Temperature range | 0° C. to 250° C. or 300° C. | |
| Scan rate | 10 | K/min |
| Nitrogen flow | 50 | mL/min |

| | |
|---|---|
| Sample mass | Approximately 2 mg |

### Dynamic Vapor Sorption (DVS) Method

DVS

| | |
|---|---|
| Instrument | Advantage or Intrinsic |
| Sample mass | approximately 10 mg |
| Temperature | 25° C. or 40° C. |
| Program | 40 - 0 - 95 - 0 - 40% RH with dm/dt = 0.002%/min |

### Solubility Studies

Solubility was measured at room temperature (RT, 20-24° C.) by the addition method and the gravimetric method.

For the addition method, approximately 20 mg of solid was added to a 2 mL vial, followed by slow addition of the respective solvent until complete dissolution was observed.

For the gravimetric method, approximately 30 mg solid was added to a 2 mL vial followed by 0.75 mL of solvent. Slurries were stirred at a constant temperature for two days. After stirring for two days, the slurries were syringe filtered, and supernatant was transferred to tared vials. Supernatant solutions were evaporated to dryness at 50° C. in atmosphere on a hot plate, then placed at 50° C. under vacuum for 3 h at approximately-29 inHg before final weighing.

### PBS-V1+3 mM NaTC

Purchase sodium taurocholate (NaTC) from supplier. Account for assay. Add amount corresponding to 3 mM to PBS V1. Stir until dissolved. Equilibrate 1 h at 25° C. or 37° C. before use. Dispose after 48 h.

### PBS-V1+FaSSIF-V2 (Fasted State Simulated Intestinal Fluid)

Purchase FaSSIF-V2 from supplier (biorelevant.com). Amount added to PBS-V1 according to Table 1. Stir until dissolved. Equilibrate 1 h at 25° C. or 37° C. before use. Dispose after 48 h.

### PBS-V1+0.3 FeSSIF-V2 (Fed State Simulated Intestinal Fluid)

Purchase FeSSIF-V2 from supplier (biorelevant.com). Amount added to PBS-V1 according to Table 1. Stir until dissolved. Equilibrate 1 h at 25° C. or 37° C. before use. Dispose after 48 h.

### PBS-V1+FeSSIF-V2 (Fed State Simulated Intestinal Fluid)

Purchase FeSSIF-V2 from supplier (biorelevant.com). Amount added to PBS-V1 according to Table 1. Stir until dissolved. Equilibrate 1 h at 25° C. or 37° C. before use. Dispose after 48 h.

### PBS-V1+2 FeSSIF-V2 (Fed State Simulated Intestinal Fluid)

Purchase FeSSIF-V2 from supplier (biorelevant.com). Amount added to PBS-V1 according to Table 1. Stir until dissolved. Equilibrate 1 h at 25° C. or 37° C. before use. Dispose after 48 h.

TABLE 1
Summary of biorelevant media composition

| Solvent medium | SIF powder (g/L) | pH |
|---|---|---|
| FaSSGF | 0.0597 | 1.6 |
| PBS-V1 + 3 mM NaTC | 1.68 | 6.5 |
| PBS-V1 + FaSSIF-V2 | 1.79 | 6.5 |
| PBS-V1 + 0.3 FeSSIF-V2 | 2.928 | 6.5 |
| PBS-V1 + FeSSIF-V2 | 9.76 | 6.5 |
| PBS-V1 + 2 FeSSIF-V2 | 19.52 | 6.5 |

### ABBREVIATIONS

ACN: Acetonitrile
THF: Tetrahydrofuran
MeOH: Methanol
EtOH: Ethanol
EA or EtOAc: Ethyl acetate
DMSO: Dimethyl sulfoxide
IPAc: Isopropyl Acetate
MEK: Methyl ethyl ketone
MTBE: tert-Butyl methyl ether
2-MeTHF: 2-Methyltetrahydrofuran
MIBK: Methyl isobutyl ketone
NaTC: Sodium Taurocholate
NMP: N-Methyl-2-pyrrolidone
DCM: Dichloromethane
XRPD: X-Ray Powder Diffraction
TSA: p-Toluene sulfonic acid
Tosylate: p-Toluene sulfonic acid salt
FaSSGF: Fasted State Simulated Gastric Fluid
FeSSIF: Fed State Simulated Intestinal Fluid
FaSSIF: Fasted State Simulated Intestinal Fluid
Kleptose: Hydroxypropyl β-cyclodextrin
K-value: Vapor-liquid equilibrium ratio
Methocel: Hydroxypropyl Methylcellulose
D₅₀: Mean particle size diameter determined from particle size distribution

### Experimental Procedures and Characterization Data

### Form A

### Preparation of Amorphous and Form A Crystalline (S)-4-(2,2-difluoro-7-((5-methoxy-7-methyl-1H-indol-4-yl)methyl)-7-azaspiro[3.5]nonan-6-yl)benzoic acid

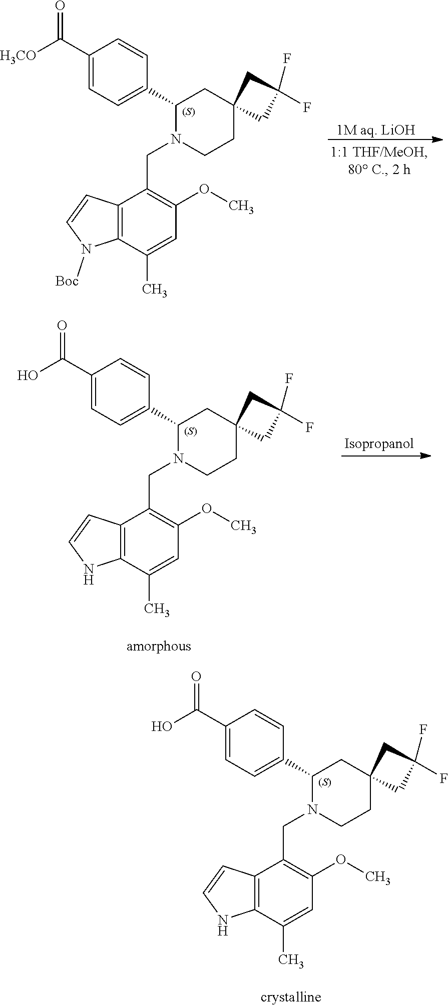

To a solution of tert-butyl (S)-4-((2,2-difluoro-6-(4-(methoxycarbonyl)phenyl)-7-azaspiro[3.5]nonan-7-yl)methyl)-5-methoxy-7-methyl-1H-indole-1-carboxylate (1.0 equiv) in 1:1 v/v THF/MeOH (0.09 M) was added 1 M aqueous LiOH solution (5.0 equiv). This mixture was heated to 45° C. with stirring for 16 hours. The resulting reaction mixture was diluted with water (to 0.1 M), concentrated under reduced pressure to one volume, and then acidified with citric acid (5% aqueous) to a pH of 6. The resulting suspension was filtered, washed with one volume of water and then dried to yield an off-white solid (87% yield) as an amorphous compound. FIG. 1 is an X-ray powder diffractogram of amorphous (S)-4-(2,2-difluoro-7-((5-methoxy-7-methyl-1H-indol-4-yl)methyl)-7-azaspiro[3.5]nonan-6-yl)benzoic acid.

The amorphous solid was added to a stirred solution of isopropanol (to 0.44 M) at 40-50° C. After the addition is complete, the solution was allowed to cool to 20° C. and the resulting suspension was stirred for 72 hours. The suspension was filtered, washed with one volume of isopropanol and then dried to yield an off-white crystalline solid (94% yield). ¹H-NMR (400 MHz, DMSO-d₆): δ 10.82 (s, 1H), 7.97 (d, J=7.9 Hz, 2H), 7.68 (d, J=7.8 Hz, 2H), 7.25 (t, J=2.9 Hz, 1H), 6.65 (s, 1H), 6.45 (s, 1H), 3.70 (s, 3H), 3.53 (d, J=11.8 Hz, 1H), 3.23 (s, 1H), 3.16 (d, J=11.9 Hz, 1H), 2.67 (d, J=12.4 Hz, 1H), 2.59 (d, J=13.1 Hz, 1H), 2.46 (s, 1H), 2.43 (s, 3H), 2.29 (t, J=13.3 Hz, 2H), 1.98 (s, 1H), 1.70 (d, J=9.0 Hz, 2H), 1.53 (d, J=9.5 Hz, 2H). LCMS (ESI) m/z 455 (M+1)+. The X-ray powder diffractogram of the Form A crystalline (S)-4-(2,2-difluoro-7-((5-methoxy-7-methyl-1H-indol-4-yl)methyl)-7-azaspiro[3.5]nonan-6-yl)benzoic acid obtained is depicted in FIG. 2.

TABLE 2
XRPD Peak List for Form A.

| Angle | d-Spacing | |
|---|---|---|
| (°2θ) | (Å) | Relative intensity |
| 5.3 | 16.5 | 10 |
| 10.7 | 8.3 | 100 |
| 11.3 | 7.8 | 5 |
| 12.5 | 7.10 | 22 |
| 13.7 | 6.5 | 10 |
| 15.5 | 5.7 | 11 |
| 15.6 | 5.7 | 8 |
| 16.1 | 5.5 | 7 |
| 16.5 | 5.3 | 10 |
| 18.8 | 4.7 | 39 |
| 19.6 | 4.5 | 37 |
| 19.8 | 4.5 | 23 |
| 20.5 | 4.3 | 88 |
| 21.1 | 4.2 | 22 |
| 21.7 | 4.1 | 38 |
| 22.5 | 3.9 | 16 |
| 23.3 | 3.8 | 18 |
| 23.5 | 3.8 | 8 |
| 25.6 | 3.5 | 4 |
| 26.7 | 3.3 | 5 |
| 27.3 | 3.3 | 12 |
| 28.9 | 3.1 | 7 |
| 29.2 | 3.0 | 4 |

The thermogravimetric analysis thermogram is depicted in FIG. 3. The differential scanning calorimetry pattern is shown in FIG. 4.

### Alternative Preparation of Form A Crystalline (S)-4-(2,2-difluoro-7-((5-methoxy-7-methyl-1H-indol-4-yl)methyl)-7-azaspiro[3.5]nonan-6-yl)benzoic acid

Approximately 3.0 g of amorphous tert-butyl (S)-4-((2,2-difluoro-6-(4-(methoxycarbonyl)phenyl)-7-azaspiro[3.5]nonan-7-yl)methyl)-5-methoxy-7-methyl-1H-indole-1-carboxylate was weighed into a 50 mL EasyMax reactor and then 20 mL of MeOH was charged. The suspension was heated to 50° C. and allowed to stir for 20 min with a stirring speed of 500 rpm, and then seeded with previously made Form A crystals. After holding 50° C. for 3 h, the suspension was cooled to 25° C. in 250 min, followed by heating to 50° C. in 30 min. After aging at 50° C. again for 3 h, the suspension was cooled to 5° C. and stirred overnight. The suspension was filtered and the filter cake was dried at 50° C. under vacuum overnight. 2.4 g of dried solids are obtained with a yield of 80%. Molecular formula of anhydrous free form C₂₆H₂₈F₂N₂O₃ (454.52 g/mol). The ¹H NMR (400 MHz, DMSO-d₆) of the solid is depicted in FIG. 8.

The X-ray powder diffractogram of the Form A (alternative preparation) is depicted in FIG. 5.

The differential scanning calorimetry pattern of Form A (alternative preparation) is shown in FIG. 6. The thermogravimetric analysis thermogram of Form A (alternative preparation) is depicted in FIG. 7.

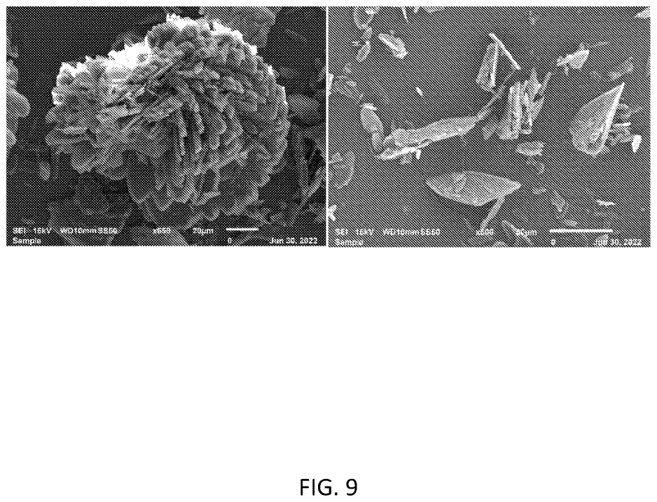

Form A shows plate like morphology as in FIG. 9.

### Form B

### Preparation of Form B (S)-4-(2,2-difluoro-7-((5-methoxy-7-methyl-1H-indol-4-yl)methyl)-7-azaspiro[3.5]nonan-6-yl)benzoic acid p-toluene sulfonic acid salt

Amorphous (S)-4-(2,2-difluoro-7-((5-methoxy-7-methyl-1H-indol-4-yl)methyl)-7-azaspiro[3.5]nonan-6-yl)benzoic acid (5 g) was charged to a 100 mL EasyMax vessel, followed by p-toluene sulfonic acid monohydrate (TSA·H₂O) (2.30 g, 1.1 eq.). Ethanol (EtOH) (17.5 mL, 3.5 vol.) was then added, and the resulting suspension was agitated (240 rpm) and heated to 50° C. for 15 min, then cooled to 25° C. over 15 min, resulting in a clear, dark-brown solution. Ethyl acetate (12.5 mL, 2.5 vol.) was added, then allowed to stand for 30 min at 25° C. Ethyl acetate (37.5 mL, 7.5 vol.) was then dosed to the reactor over 2 hours through a dosing pump. After completion of the anti-solvent addition, the purple slurry was then allowed to stand for 1 h at 25° C.

The final slurry was filtered, washed twice with 2 volumes of EtOH: EtOAc (26:74 v/v), and pulled dry until a transferrable powder was obtained. Filtration was expedient, with a K-value of 10.4 cm2/(min·bar)).

The solids were transferred to a tared bottle, and dried for 16 h in a 50° C. vacuum oven (approx. −29 in Hg). Solids were isolated in a 79 molar % yield.

Form B was prepared by heating the input tosylate (300 mg) in IPA: water (9:1 vol., 1.5 mL) at 50° C. for 16 h. The resulting slurry was sampled for XRPD, then cooled to room temperature and filtered. Resulting wet cake solids were dried in a 50° C. vacuum oven under active vacuum. The yield was 237 mg (79% w/w).

The X-ray powder diffractogram of Form B is shown in FIG. 12, and the XRPD peak table is shown below.

TABLE 3
XRPD Peak List for Form B

| Angle | d-Spacing | Relative |
|---|---|---|
| (°2θ) | (Å) | intensity |
| 9.3 | 9.5 | 70 |
| 10.2 | 8.7 | 38 |
| 11.8 | 7.5 | 100 |
| 13.8 | 6.4 | 7 |
| 14.2 | 6.2 | 23 |
| 15.3 | 5.8 | 9 |
| 15.4 | 5.7 | 15 |
| 16.9 | 5.2 | 7 |
| 17.2 | 5.1 | 46 |
| 18.2 | 4.9 | 17 |
| 18.5 | 4.8 | 7 |
| 18.7 | 4.7 | 7 |
| 19.9 | 4.5 | 66 |
| 20.2 | 4.4 | 11 |
| 20.4 | 4.3 | 37 |
| 20.7 | 4.3 | 16 |
| 21.3 | 4.2 | 27 |
| 22.9 | 3.9 | 46 |
| 23.7 | 3.8 | 9 |
| 24.4 | 3.6 | 14 |
| 25.2 | 3.5 | 8 |

The thermogravimetric analysis thermogram is depicted in FIG. 13. The differential scanning calorimetry pattern is shown in FIG. 14.

### Alternative Preparation I of Form B (S)-4-(2,2-difluoro-7-((5-methoxy-7-methyl-1H-indol-4-yl)methyl)-7-azaspiro[3.5]nonan-6-yl)benzoic acid p-toluene sulfonic acid salt

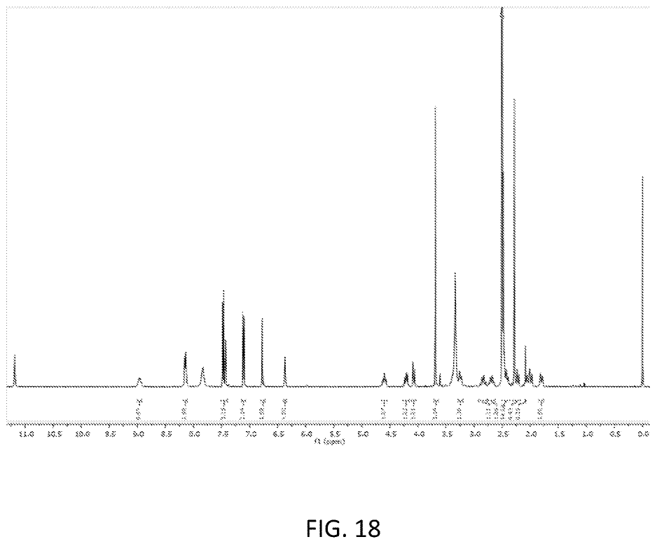

Approximately 10 g of (S)-4-(2,2-difluoro-7-((5-methoxy-7-methyl-1H-indol-4-yl)methyl)-7-azaspiro[3.5]nonan-6-yl)benzoic acid and 1.05 eq of p-TsOH monohydrate (˜4.45 g) were weighed into a 400 mL EasyMax reactor. 10 mL of water and 90 mL of acetone were added to the reactor and the solution was heated to 50° C. The solution was stirred for 20 min with a speed of 300 rpm then cooled to 25° C. The solution was seeded with 50 mg of previously prepared Form B crystals. The temperature was held for 1 h, followed by dosing 220 mL of water within 5 h. After holding for 2 h, the reaction was cooled to 5° C. in 2 h and stirred overnight. The suspension was filtered and the filter cake was dried at 40° C. under vacuum for 24 hour to yield 11.59 g Form B (S)-4-(2,2-difluoro-7-((5-methoxy-7-methyl-1H-indol-4-yl)methyl)-7-azaspiro[3.5]nonan-6-yl)benzoic acid p-toluene sulfonic acid salt (80%). ¹H-NMR (400 MHz, DMSO-d₆): δ 11.19 (s, 1H), 8.14 (d, 2H), 7.84 (d, 1H), 7.49 (m, 3H), 7.11 (d, 2H), 6.77 (s, 1H), 6.39 (t, 1H), 4.63 (m, 1H), 4.23 (m, 1H), 4.08 (d, 1H), 3.69 (s, 3H), 3.41 (m, 2H), 2.67-2.82 (m, 2H), 2.48 (s, 3H), 2.45 (m, 2H), 2.28 (s, 3H), 1.98-2.23 (m, 2H), 1.79 (d, 2H) (FIG. 18). Molecular formula of anhydrous tosylate salt C₂₆H₂₈F₂N₂O₃·C₇H₈O₃S (626.72 g/mol).

The X-ray powder diffractogram of Form B (alternative preparation) obtained is depicted in FIG. 15.

TABLE 4
XRPD Peak List for Form B (alternative preparation I)

| | | | |
|---|---|---|---|
| Angle | d-Spacing | Relative | |
| (°2θ) | (Å) | intensity | Rel. intensity rank |

| | | | | |
|---|---|---|---|---|
| 9.280° | 9.52241 | Å | 51.6% | 3 |
| 10.148° | 8.70982 | Å | 39.1% | 4 |
| 18.151° | 4.88347 | Å | 15.2% | 10 |
| 11.790° | 7.50015 | Å | 100.0% | 1 |
| 14.185° | 6.23850 | Å | 21.1% | 8 |
| 15.428° | 5.73861 | Å | 10.8% | 12 |
| 17.187° | 5.15508 | Å | 36.2% | 6 |
| 19.864° | 4.46597 | Å | 55.9% | 2 |
| 20.364° | 4.35744 | Å | 23.8% | 7 |
| 20.696° | 4.28836 | Å | 10.1% | 13 |
| 21.278° | 4.17232 | Å | 18.4% | 9 |
| 22.915° | 3.87778 | Å | 38.4% | 5 |
| 24.396° | 3.64563 | Å | 7.4% | 15 |
| 25.549° | 3.48375 | Å | 15.2% | 11 |
| 27.633° | 3.22558 | Å | 10.1% | 14 |

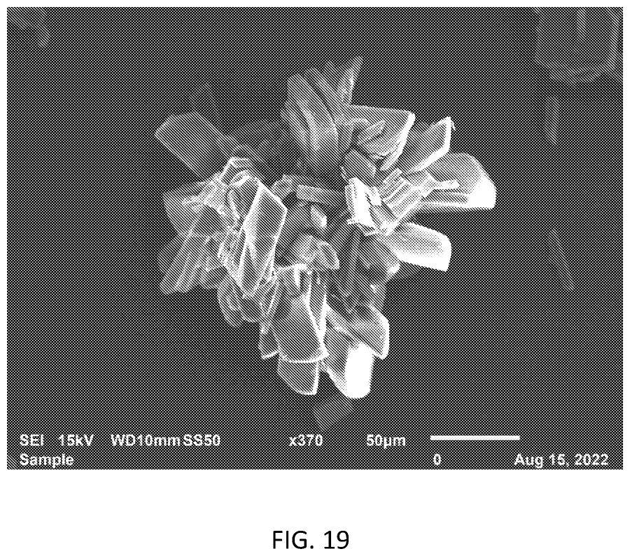

The thermogravimetric analysis thermogram is depicted in FIG. 17. The differential scanning calorimetry pattern is shown in FIG. 16. Form B shows plate like morphology as in FIG. 19.

### Alternative Preparation II of Form B (S)-4-(2,2-difluoro-7-((5-methoxy-7-methyl-1H-indol-4-yl)methyl)-7-azaspiro[3.5]nonan-6-yl)benzoic acid p-toluene sulfonic acid salt

2.0 g of (S)-4-(2,2-difluoro-7-((5-methoxy-7-methyl-1H-indol-4-yl)methyl)-7-azaspiro[3.5]nonan-6-yl)benzoic acid and 0.89 g of p-toluene sulfonic acid were weighed into a 100 mL reactor. 16.2 g of acetone/water (9/1, v/v) was added and the solution was stirred at 25° C. with a stirring speed of 250 rpm. 10 mL water was added and then 5 mg of seed was added and held for 2 hours. An additional 30 mL of water was added over 4 h and then the solution was cooled to 5° C. in 2 h. After holding at 5° C. overnight, the suspension was filtered and the filter cake was dried at 50° C. under vacuum overnight. 2.2 g of dried solids are obtained with a yield of 80%.

### Water Content in Forms A and B

Form B contained 2.3% w/w water content relative to the total weight of the tosylate salt, compared to 0.15% w/w in Form A relative to the total weight of the free form, by Karl Fischer titration under ambient conditions.

The mass change by DVS isotherm (2 cycles 40-0-95-0-40% RH) are in the tables below. The mass change is based on the dry weight of each.

TABLE 5
Mass change by DVS of Forms A and B at 25 degree C.

| | Form A | | Form B | |
|---|---|---|---|---|
| % RH | Sorption (%) | Desorption (%) | Sorption (%) | Desorption (%) |
| 0 | 0.00 | −0.01 | 0.00 | 0.00 |
| 10 | 0.04 | 0.06 | 1.08 | 1.11 |
| 20 | 0.08 | 0.12 | 1.64 | 1.66 |
| 30 | 0.13 | 0.17 | 1.92 | 1.93 |
| 40 | 0.17 | 0.22 | 2.09 | 2.10 |
| 50 | 0.22 | 0.26 | 2.21 | 2.22 |
| 60 | 0.26 | 0.32 | 2.31 | 2.32 |
| 70 | 0.30 | 0.35 | 2.38 | 2.39 |
| 80 | 0.35 | 0.38 | 2.45 | 2.46 |
| 90 | 0.40 | 0.42 | 2.54 | 2.54 |
| 95 | 0.45 | 0.45 | 2.62 | 2.62 |

The XRPD profiles were unchanged after the DVS experiments above.

The maximum water uptake of Form B is 2.67% at 95% RH at 25° C. in DVS.

TABLE 6
Mass change by DVS of Form B at 25 and 40 degree C.

| | DVS at 25 degree C. | | | | DVS at 40 degree C. | | | |
|---|---|---|---|---|---|---|---|---|
| | 1^{st} | 1^{st} | 2^{nd} | 2^{nd} | 1^{st} | 1^{st} | 2^{nd} | 2^{nd} |
| | Desorption | Sorption | Desorption | Sorption | Desorption | Sorption | Desorption | Sorption |
| % RH | (%) | (%) | (%) | (%) | (%) | (%) | (%) | (%) |
| 0 | 0.00 | 0.00 | −0.01 | −0.01 | 0.00 | 0.00 | −0.01 | −0.01 |
| 10 | 1.13 | 1.11 | 1.13 | 1.11 | 0.97 | 0.96 | 0.97 | 0.95 |
| 20 | 1.70 | 1.69 | 1.70 | 1.68 | 1.61 | 1.59 | 1.60 | 1.59 |
| 30 | 1.98 | 1.97 | 1.98 | 1.97 | 1.92 | 1.90 | 1.91 | 1.90 |
| 40 | 2.16 | 2.15 | 2.16 | 2.15 | 2.10 | 2.09 | 2.09 | 2.09 |
| 50 | | 2.28 | 2.29 | | | 2.21 | 2.22 | |
| 60 | | 2.37 | 2.38 | | | 2.28 | 2.29 | |
| 70 | | 2.45 | 2.47 | | | 2.34 | 2.37 | |
| 80 | | 2.53 | 2.54 | | | 2.40 | 2.44 | |
| 90 | | 2.60 | 2.61 | | | 2.49 | 2.49 | |
| 95 | | 2.67 | 2.67 | | | 2.52 | 2.52 | |

### Stability of Forms A and B

In bulk state, Form A is chemically stable when stored at 50° C. with 11% RH or 75% RH for 1 week. When stored at 80° C. with 11% RH or 75% RH, there was about 0.1% to 0.3% degradation, and slight to medium discoloration was observed. Form A is chemically and physically stable when stored in HPMC and HGC capsules at 50° C. with 11% and 75% RH for 2 weeks. When exposed under 1200 kLux light stress, there was about 0.9% degradation. The polymorphic form remained unchanged in the stress conditions.

In bulk state, Form B is chemically stable when stored at 50° C. with 11% RH, 50° C./75% RH and 80° C./11% RH for 1 week. However, when stored at 80° C. with 75% RH, there was about 0.1% degradation and slight discoloration was observed. Form B is chemically and physically stable when stored in HPMC and HGC capsules at 50° C. with 11% and 75% RH for 2 weeks. When exposed under 1200 kLux light stress, there was about 0.2% degradation with slight discoloration observed. The form remained no change in the stress conditions.

When exposed to 80% or 92% RH for 24 hrs, there was no change in Form A or B by XRPD.

### Solubility of Forms A and B

The solubility of Forms A and B in water and biorelevant buffers at 25° C. was collected at 24 h. Form B showed significantly increased solubility at 24 h in all media over Form A.

TABLE 7
Solubility (mg/mL) in water and biorelevant buffers at 25° C.

Sample

| | Form A | Form B |
|---|---|---|
| | S (pH) | S (pH) |
| Medium | XRPD | XRPD |
| water | 0.052 (7.40) | 0.39 (3.89) |
| | — | — |
| FaSSGF, pH 1.6 | >2 (1.68) | 0.49 (1.77) |
| | // | No change |
| PBS-V1 + 3 mM | 0.068 (6.51) | >2 (6.42) |
| NaTC, pH 6.5 | — | // |
| PBS-V1 + FaSSIF- | 0.072 (6.69) | >2 (6.35) |
| V2, pH 6.5 | — | // |
| PBS-V1 + 0.3 | 0.074 (6.52) | 0.10 (6.12) |
| FeSSIF-V2, pH 6.5 | — | Form A |
| PBS-V1 + FeSSIF- | 0.094 (6.74) | 0.20 (6.40) |
| V2, pH 6.5 | — | Form A |
| PBS-V1 + 2.0 | 0.12 (6.73) | 0.31 (6.19) |
| FeSSIF-V2, pH 6.5 | — | Form A |

“//”: not carried out due to clear solution or limited residual material
“—”: no form change

### Intrinsic Dissolution of Forms A and B

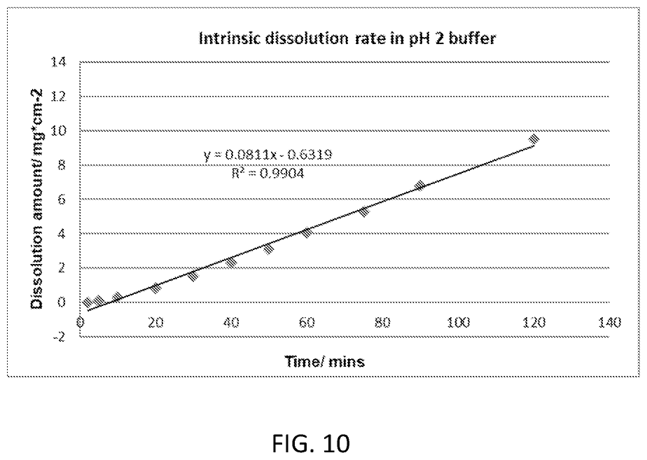

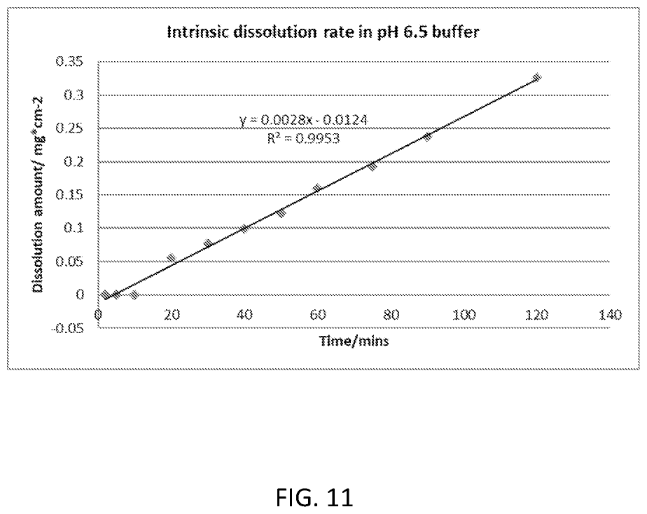

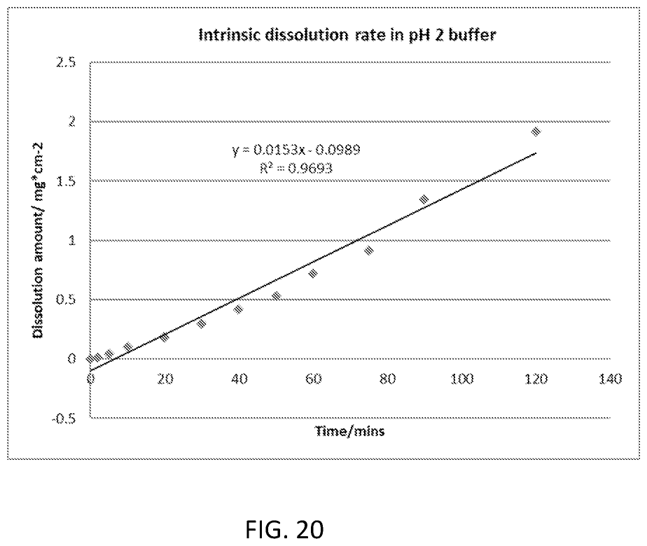

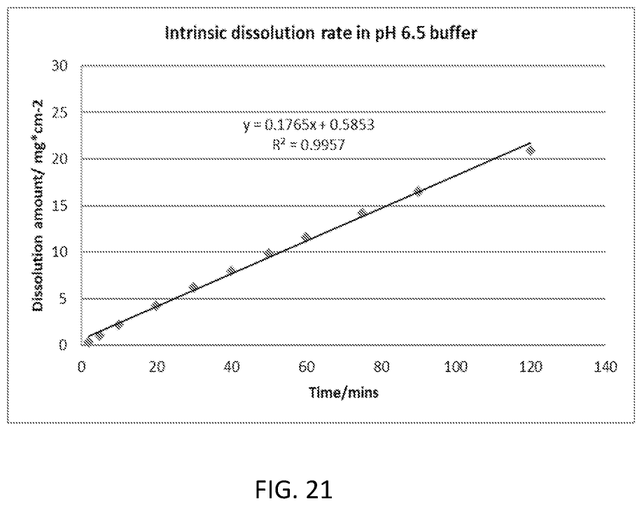

The intrinsic dissolution rate of Form A in pH 2.0, 0.01 M HCl is 0.081 mg/min/cm² (FIG. 10), which is higher than that of Form B at 0.015 mg/min/cm² in the same media (FIG. 20). In PBS-V1 (pH 6.5), the intrinsic dissolution rate of Form A is 0.0028 mg/min/cm² (FIG. 11), whereas Form B shows a much higher dissolution rate of 0.18 mg/min/cm² (FIG. 21).

Intrinsic dissolution rate at 37° C., 100 rpm
(mg · min⁻¹ · cm⁻²), 120 min

| | Form A | Form B |
|---|---|---|
| pH 2.0, 0.01 M HCl | 0.081 | 0.015 |
| | (No form change) | (No form change) |
| pH 6.5, phosphate buffer | 0.0028 | 0.18 |
| 50 mM (PBS-V1) | (No form change) | (No form change |

### Compression Stability of Forms A and B

There was no form change in XRPD when Form A or B was subjected to 4 tons of compression for 5 min.

Method: Compress about 100 mg of drug substance for 5 min at 4 t with a hydraulic press (diameter of the tablets 8 mm). Afterwards, characterize the sample by XRPD to detect any change in the solid state.

### Grinding and Wet Granulation of Forms A and B

Dry grinding (2 min and 5 min) and wet granulation (water and ethanol) did not lead to any form change in Forms A and B. However, crystallinity decrease was observed from the XRPD pattern after grinding.

Method: Granulate was obtained in solid-state forms. Dropwise add granulating solvent until the solid is wetted sufficiently. Grind the wet solid for about 2 min. Dry the wet-cake under vacuum or at atmospheric pressure. Evaluate solid form and degree of crystallinity by e.g. XRPD and/or DSC.

### Flow Properties of Form B

Form B had a good flowability and a good bulk density which are suitable for both precision drug processing filler configurations (sonication and vibrational module). See U.S. Pat. No. 11,642,315.

### Dissolution of Form B in Capsule

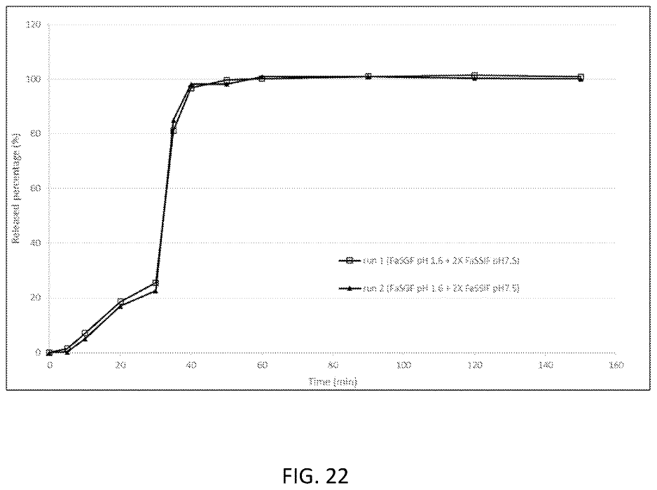

A capsule containing Form B showed 100% dissolution in 40 min in FaSSGF and FeSSIF (FIG. 22). The particle size of the material used was characterized as 53.2 μm (×50). The test conditions are as below:
Dose/Strength, 25 mg (calculated as free form)
HGC capsule, 3 #
450 mL FaSSGF (pH 1.6)+450 mL FaSSIF (2×, pH 7.5)
Temperature 37° C.
Peddle, 75 rpm
Spiral sinker
FaSSIF (2×, pH 7.5) added at 30 min
Duplicate tests

### Summary of Forms A and B

Forms A and B showed desirable properties in all aspects. However, Form B provided improved solubility and dissolution profiles, and flow properties amenable to a capsule formulation approach.

To prevent the possible formation of esters of toluenesulfonic acid which are known to exert genotoxic effects, the use of methanol, ethanol or any other alcohol during the manufacture of the drug substance or the drug product should be carefully avoided.

### Form C

### Preparation of Form C (S)-4-(2,2-difluoro-7-((5-methoxy-7-methyl-1H-indol-4-yl)methyl)-7-azaspiro[3.5]nonan-6-yl)benzoic acid p-toluene sulfonic acid salt

Amorphous (S)-4-(2,2-difluoro-7-((5-methoxy-7-methyl-1H-indol-4-yl)methyl)-7-azaspiro[3.5]nonan-6-yl)benzoic acid (5 g) and p-toluene sulfonic acid monohydrate (TSA·H₂O) (2.30 g, 1.1 eq.) were charged to a 100 mL EasyMax vessel. Ethanol (17.5 mL, 3.5 vol.) was then added, and the resulting suspension was heated to 50° C. with agitation (240 rpm) for 15 min, then cooled to 25° C. over 15 min. To the resulting clear, dark-brown solution was added EtOAc (12.5 mL, 2.5 vol.), then allowed to stand for 30 min at 25° C. EtOAc (37.5 mL, 7.5 vol.) was then added to the reactor over 2 h using a dosing pump. After completion of the anti-solvent addition, the purple slurry was then aged for 1 h at 25° C. The final slurry was filtered, washed 2×2 volumes of EtOH: EtOAc (26:74 vol), and pulled dry until a transferrable powder was obtained.

FIG. 23 is an X-ray powder diffractogram of Form C, and the XRPD peak table is shown below.

TABLE 8
XRPD Peak List for Form C (S)-4-(2,2-difluoro-7-((5-methoxy-
7-methyl-1H-indol-4-yl)methyl)-7-azaspiro[3.5]nonan-
6-yl)benzoic acid p-toluene sulfonic acid salt.

| Angle | d-Spacing | Relative |
|---|---|---|
| (°2θ) | (Å) | intensity |
| 5.3 | 16.5 | 24 |
| 9.5 | 9.3 | 13 |
| 10.2 | 8.6 | 40 |
| 10.7 | 8.3 | 35 |
| 11.3 | 7.8 | 23 |
| 11.5 | 7.7 | 56 |
| 13.3 | 6.7 | 15 |
| 13.6 | 6.5 | 12 |
| 15.3 | 5.8 | 41 |
| 16.3 | 5.4 | 11 |
| 17.3 | 5.1 | 76 |
| 17.5 | 5.1 | 74 |
| 18.2 | 4.9 | 17 |
| 18.6 | 4.8 | 20 |
| 19.1 | 4.6 | 13 |
| 19.4 | 4.6 | 11 |
| 19.7 | 4.5 | 20 |
| 19.8 | 4.5 | 31 |
| 20.4 | 4.3 | 17 |
| 21.5 | 4.1 | 15 |
| 21.8 | 4.1 | 65 |
| 22.3 | 4.0 | 100 |
| 22.6 | 3.9 | 10 |
| 23.2 | 3.8 | 7 |
| 24.1 | 3.7 | 17 |
| 24.5 | 3.6 | 10 |
| 24.7 | 3.6 | 17 |
| 25.6 | 3.5 | 15 |
| 26.6 | 3.3 | 32 |
| 27.3 | 3.3 | 11 |
| 28.8 | 3.1 | 6 |
| 29.3 | 3.0 | 5 |
| 30.9 | 2.9 | 5 |
| 37.0 | 2.4 | 6 |
| 37.9 | 2.4 | 10 |
| 39.6 | 2.3 | 8 |

The thermogravimetric analysis thermogram is depicted in FIG. 24. The differential scanning calorimetry pattern is shown in FIG. 25.

### Alternative Preparation of Form C (S)-4-(2,2-difluoro-7-((5-methoxy-7-methyl-1H-indol-4-yl)methyl)-7-azaspiro[3.5]nonan-6-yl)benzoic acid p-toluene sulfonic acid salt

1 g of Form B was dissolved into 3 mL MeOH, and then the solution was added to 15 mL EA. The suspension was filtered after a few hours and the filter cake was vacuum dried at 40° C. overnight to yield about 650 mg of Form C (65%).

The X-ray powder diffractogram of the Form C is consistent with FIG. 23.

### Polymorphic Stability

Competitive equilibrations between Form B and Form C showed that Form B is the stable polymorphic form in all the tested solvents or solvent mixtures.

### Form D

### Preparation of Form D (S)-4-(2,2-difluoro-7-((5-methoxy-7-methyl-1H-indol-4-yl)methyl)-7-azaspiro[3.5]nonan-6-yl)benzoic acid hydrochloride salt

To a solution of amorphous (S)-4-(2,2-difluoro-7-((5-methoxy-7-methyl-1H-indol-4-yl)methyl)-7-azaspiro[3.5]nonan-6-yl)benzoic acid in EtOAc (5.5 vol.) was added a portion of 1 M HCl in EtOAc (0.55 equiv.), followed by a seed slurry, resulting in a grey suspension. The remaining 1 M HCl in EtOAc (1.65 equiv.) was then added in a single portion, resulting in a thick, grey slurry. The resulting thick slurry was stirred at 24° C. and aged for 16 h. The resulting slurry was filtered, and top washed 2×1.5 volumes of EtOAc. The filter cake was dried under vacuum to afford an off-white solid (71% yield). The X-ray powder diffractogram of Form D is shown in FIG. 26. The thermogravimetric analysis thermogram is depicted in FIG. 27. The differential scanning calorimetry pattern is shown in FIG. 28.

TABLE 9
XRPD Peak List for Form D.

| Angle | d-Spacing | Relative |
|---|---|---|
| (°2θ) | (Å) | intensity |
| 8.2 | 10.7 | 27 |
| 9.6 | 9.2 | 11 |
| 9.9 | 8.9 | 11 |
| 10.4 | 8.5 | 93 |
| 11.0 | 8.0 | 9 |
| 11.2 | 7.9 | 8 |
| 12.4 | 7.1 | 10 |
| 13.1 | 6.8 | 100 |
| 14.9 | 5.9 | 9 |
| 15.2 | 5.8 | 24 |
| 15.7 | 5.6 | 21 |
| 15.9 | 5.6 | 61 |
| 16.4 | 5.4 | 100 |
| 16.6 | 5.3 | 79 |
| 17.5 | 5.0 | 49 |
| 18.2 | 4.9 | 64 |
| 19.7 | 4.5 | 16 |
| 19.9 | 4.4 | 25 |
| 20.6 | 4.3 | 43 |
| 20.9 | 4.2 | 9 |
| 22.4 | 4.00 | 27 |
| 22.6 | 3.9 | 14 |
| 23.4 | 3.8 | 73 |
| 24.1 | 3.7 | 37 |
| 24.4 | 3.6 | 10 |
| 24.9 | 3.6 | 56 |
| 25.4 | 3.5 | 11 |
| 26.0 | 3.4 | 8 |
| 26.3 | 3.4 | 12 |
| 26.8 | 3.3 | 16 |
| 27.3 | 3.3 | 22 |
| 27.9 | 3.2 | 28 |
| 30.3 | 2.9 | 8 |
| 31.6 | 2.8 | 9 |
| 32.5 | 2.7 | 6 |
| 33.2 | 2.7 | 8 |
| 34.0 | 2.6 | 6 |
| 35.3 | 2.5 | 5 |
| 39.3 | 2.3 | 5 |

### Solubility Studies

Solubility data of Form A (S)-4-(2,2-difluoro-7-((5-methoxy-7-methyl-1H-indol-4-yl)methyl)-7-azaspiro[3.5]nonan-6-yl)benzoic acid, Form B (S)-4-(2,2-difluoro-7-((5-methoxy-7-methyl-1H-indol-4-yl)methyl)-7-azaspiro[3.5]nonan-6-yl)benzoic acid p-toluene sulfonic acid salt, Form C (S)-4-(2,2-difluoro-7-((5-methoxy-7-methyl-1H-indol-4-yl)methyl)-7-azaspiro[3.5]nonan-6-yl)benzoic acid p-toluene sulfonic acid salt, and Form D (S)-4-(2,2-difluoro-7-((5-methoxy-7-methyl-1H-indol-4-yl)methyl)-7-azaspiro[3.5]nonan-6-yl)benzoic acid hydrochloride salt in Simulated Fluids at 37° C.

TABLE 10

| | | | | Solubility (mg/mL) | |
|---|---|---|---|---|---|
| Input Pattern | Fluid | Initial pH | pH at 24 h | 30 min | 24 h |
| Form A | FaSSGF | 1.58 | 1.61 | 2.28 | >2 |
| | FeSSIF | 5.03 | 5.00 | 0.31 | 0.31 |
| | FaSSIF | 6.49 | 6.48 | 0.24 | 0.18 |
| | Water | 7.00 | 7.12 | 0.21 | 0.22 |
| Form B | FaSSGF | 1.63 | 1.5 | 0.40 | 0.54 |
| | FeSSIF | 4.95 | 4.87 | >2 | >2 |
| | FaSSIF | 6.46 | 6.13 | >2 | 0.53 (salt |
| | | | | | dissociates to |
| | | | | | Form A as |
| | | | | | determined by |
| | | | | | XRPD) |
| | Water | 7.00 | 3.60 | 0.48 | 0.56 |
| Form C | FaSSGF | 1.58 | 1.56 | 0.87 | 0.85 |
| | FeSSIF | 5.03 | 4.97 | 1.63 | 1.69 |
| | FaSSIF | 6.49 | 6.25 | 1.68 | 0.13 |
| | Water | 7.00 | 3.37 | 0.88 | 1.12 |
| Form D | FaSSGF | 1.58 | 1.57 | 2.25 | >2 |
| | FeSSIF | 5.03 | 4.95 | 2.19 | 2.33 |
| | FaSSIF | 6.49 | 6.17 | 2.19 | >2 |
| | Water | 7.00 | 3.13 | 2.08 | >2 |

### Pharmacokinetic Studies

The pharmacokinetic profile of (S)-4-(2,2-difluoro-7-((5-methoxy-7-methyl-1H-indol-4-yl)methyl)-7-azaspiro[3.5]nonan-6-yl)benzoic acid was assessed in rats, dogs or non-human primates (NHPs) as the amorphous compound as a solution in 20% kleptose in water and as a suspension of Form A in 0.5% methocel+0.1% tween 80 in water.

### Preparation of Amorphous Solution Formulation

Amorphous (S)-4-(2,2-difluoro-7-((5-methoxy-7-methyl-1H-indol-4-yl)methyl)-7-azaspiro[3.5]nonan-6-yl)benzoic acid was weighed into a glass vial with a magnetic stir bar to give formulations at dose concentrations of 0.1-1000 mg/kg. Approx. ⅓ the final volume of 20% kleptose (hydroxypropyl β-cyclodextrin, Aldrich, CAS #128446-35-5) in water was added to the vial (to afford either a 5 mL/kg or 10 mL/kg dose volume, depending on the species). The mixture was basified to a pH of 8.5 to 10 using dropwise addition of 2-5 M aqueous NaOH solution, with pH monitoring. The suspension was stirred, vortexed, and sonicated for 15-20 minutes to afford a homogenous mixture. The second ⅓ of the final volume of 20% kleptose was added to the vial and further stirred, vortexed, and sonicated for 15-20 minutes to afford a homogenous mixture. The pH was monitored and further pH adjustment to pH 8.5 to 10, if necessary, can be applied. The final ⅓ the final volume of 20% kleptose was added to the vial and further stirred, vortexed, and sonicated for 15-20 minutes to afford a homogenous solution. The pH of the solution was adjusted to a pH of 7.5 to 8.0 with dropwise addition of 1.0 M aqueous HCl solution with gentle stirring. The formulation should remain in solution.

Table 10 describes the pharmacokinetic properties of the amorphous solution formulation after oral dosing.

TABLE 11
Pharmacokinetic Properties of Amorphous (S)-4-(2,2-difluoro-7-((5-methoxy-
7-methyl-1H-indol-4-yl)methyl)-7-azaspiro[3.5]nonan-6-yl)benzoic acid.

| Species/ | | | Fed/ | Dose | | AUC_{0-24 h} | | C_{24 h} |
|---|---|---|---|---|---|---|---|---|
| sex | Dose | | Fasted | Volume | | (μM*h) | Cₘₐₓ(μM) | (μM) |
| SD Rat | 2 | mg/kg | Fed | 10 | mL/kg | 20.5 | 2.42 | 0.21 |
| male | | | | | | | | |
| SD Rat | 300 | mg/kg | Fed | 10 | mL/kg | 1110 | 94 | 18 |
| male | | | | | | | | |
| Dog | 2 | mg/kg | Fasted | 5 | mL/kg | 54.3 | 3.64 | 1.73 |
| beagle | | | | | | | | |
| male | | | | | | | | |
| Dog | 100 | mg/kg | Fasted | 5 | mL/kg | 1320 | 139 | 18.8 |
| beagle | | | | | | | | |
| male | | | | | | | | |
| Cyno | 30 | mg/kg | Fed | 5 | mL/kg | 96.1 | 8.6 | 1.75 |
| male | | | | | | | | |

### Preparation of Form a Suspension Formulation

Form A crystalline (S)-4-(2,2-difluoro-7-((5-methoxy-7-methyl-1H-indol-4-yl)methyl)-7-azaspiro[3.5]nonan-6-yl)benzoic acid was weighed into a glass vial with a magnetic stir bar to give formulations at dose concentrations of 0.1-1000 mg/kg. The crystalline material should preferably have a particle size with a D₅₀ below 50 μm, and ideally with a D₅₀ below 5 μm. The desired volume of 0.5% methocel (viscosity 400 cP, Aldrich, CAS #9004-67-5)/0.1% tween-80 (Aldrich, CAS #9005-65-6) was added (to afford a 5 mL/kg dose volume). The contents of the vial were stirred at 400-500 rpm on a stir-plate. Gentle vortex or bath sonication can be applied to break down any large clumps. The vial should be mixed well to ensure homogeneity right before animal dosing.

Table 12 describes the pharmacokinetic properties of the crystalline suspension formulation after oral dosing.

TABLE 12
Pharmacokinetic Properties of Form A Crystalline (S)-
4-(2,2-difluoro-7-((5-methoxy-7-methyl-1H-indol-4-
yl)methyl)-7-azaspiro[3.5]nonan-6-yl)benzoic acid.

| Species/ | | | Dose | Fed/ | AUC_{0-24 h} | Cₘₐₓ | C_{24 h} |
|---|---|---|---|---|---|---|---|
| sex | Dose | | Volume | Fasted | (μM*h) | (μM) | (μM) |
| SD Rat | 30 | mg/kg¹ | 5 mL/kg | Fed | 79.2 | 6.1 | 1.9 |
| male | | | | | | | |
| SD Rat | 300 | mg/kg¹ | 5 mL/kg | Fed | 958 | 157 | 3.3 |
| male | | | | | | | |
| SD Rat | 300 | mg/kg² | 5 mL/kg | Fed | 547 | 80 | 2.7 |
| male | | | | | | | |

¹Particle size D₅₀ = 5.1 μm.
²Particle size D₅₀ = 101 μm.

Form A exhibits a higher Cₘₐₓ, AUC₀₋₂₄ and C₂₄ₕ than the amorphous form, resulting in overall higher exposures with the smaller particle size. This is due to the higher surface area from the smaller particle size.

A number of embodiments of the disclosure have been described. Nevertheless, it will be understood that various modifications may be made without departing from the spirit and scope of the present disclosure. Accordingly, other embodiments are in the claims.

What is claimed is:

1. A crystalline form of (S)-4-(2,2-difluoro-7-((5-methoxy-7-methyl-1H-indol-4-yl)methyl)-7-azaspiro[3.5]nonan-6-yl)benzoic acid.

2. The crystalline form of claim 1, wherein the crystalline form is Form A, and wherein the XRPD pattern has peaks (±0.2 degrees 2θ) at 10.7, 20.5, and 18.8.

3. The crystalline form of claim 1, wherein the crystalline form is Form A, and wherein the XRPD pattern has peaks (±0.2 degrees 2θ) at 10.7, 20.5, 18.8, and 21.7.

4. The crystalline form of claim 1, wherein the crystalline form is Form A, and wherein the XRPD pattern has peaks (±0.2 degrees 2θ) at 10.7, 20.5, 18.8, 21.7, 19.6, 19.8, 12.5, 21.1, 23.3, 22.5, 27.3, and 15.5.

5. The crystalline form of any one of claims 1-4, wherein the crystalline form is Form A having a thermogravimetric analysis (TGA) curve characterized by a weight loss of about 0.51% at 150° C. when heated from 30° C. and 300° C. at 10 K/min.

6. The crystalline form of claim 1, wherein the (S)-4-(2,2-difluoro-7-((5-methoxy-7-methyl-1H-indol-4-yl)methyl)-7-azaspiro[3.5]nonan-6-yl)benzoic acid is in a free form, and further wherein the free form is an anhydrate form.

7. A crystalline p-toluene sulfonic acid salt form of (S)-4-(2,2-difluoro-7-((5-methoxy-7-methyl-1H-indol-4-yl)methyl)-7-azaspiro[3.5]nonan-6-yl)benzoic acid.

8. The crystalline form of claim 7, wherein the crystalline form is Form B, and wherein the XRPD pattern has peaks (±0.2 degrees 2θ) at 11.80, 9.30, and 19.9.

9. The crystalline form of claim 7, wherein the crystalline form is Form B, and wherein the XRPD pattern has peaks (±0.2 degrees 2θ) at 11.8, 9.3, 19.9, and 22.9.

10. The crystalline form of claim 7, wherein the crystalline form is Form B, and wherein the XRPD pattern has peaks (±0.2 degrees 2θ) at 11.8, 9.3, 19.9, 22.9, 17.2, 10.2, 20.4, 21.3, and 14.2.

11. The crystalline form of claim 7, wherein the crystalline form is Form B, and wherein Form B comprises water in an amount from 0% to 2.7% w/w.

12. The crystalline form of any one of claims 7-11, wherein the crystalline form is Form B having a TGA curve characterized by a weight loss of about 1.9% to about 2.0% at 100° C. when heated from 30° C. and 300° C. at 10 K/min.

13. The crystalline form of any one of claims 7-11, wherein the crystalline form is Form B having a DSC thermogram characterized by a broad endotherm at Tₒₙₛₑₜ=29.8° C. and Tₚₑₐₖ=65° C. when heated from 30 and 300° C. at a rate of 10 K/min.

14. The crystalline form of any one of claims 7-11, wherein the crystalline form is Form B absorbing up to 2.7% of moisture at 95% RH at 25° C. by dynamic vapor sorption (DVS).

15. A pharmaceutical composition comprising the crystalline form of any one of claims 1-14 and a pharmaceutically acceptable carrier.

16. A method of treating a disease or disorder associated with complement factor B (CFB), comprising administering to a subject having such disease or disorder, a therapeutically effective amount of the crystalline form of any one of claims 1-14, or the pharmaceutical composition of claim 15.

17. The method of claim 16 wherein the disease or disorder associated with complement factor B (CFB) is selected from the group consisting of: age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, glaucoma, Doyne honeycomb retinal dystrophy/Malattia leventinese, Sorsby fundus dystrophy, Late onset retinal macular dystrophy, North Carolina macular dystrophy, Stargardt disease, corneal inflammation, multiple sclerosis, stroke, Guillain Barré Syndrome, spinal cord injury, traumatic brain injury, Parkinson's disease, Alzheimer's disease, schizophrenia, amyotrophic lateral sclerosis (ALS), Huntington's disease, multifocal motor neuropathy, autism spectrum disorders, schizophrenia, drug-induced neurotoxicity, hemodialysis complications, hyperacute allograft rejection, xenograft rejection, interleukin-2 induced toxicity during IL-2 therapy, inflammatory disorders, paroxysmal nocturnal hemoglobinuria, C3 glomerulonephritis (including dense deposit disease and C3 glomerulonephritis), immune complex membranoproliferative glomerulonephritis (IC-MPGN), IgA nephropathy, membranous nephropathy including idiopathic membranous nephropathy, diabetic nephropathy, atypical hemolytic uremic syndrome (aHUS), Hemolytic uremic syndrome, STEC-HUS (Shiga toxin-producing Escherichia coli hemolytic uremic syndrome), peridontitis, CD55 deficiency with hyperactivation of complement, angiopathic thrombosis, protein-losing enteropathy (CHAPLE syndrome), Crohn's disease, neuromyelitis optica (NMO), IgA vasculitis (formerly known as Henoch-Schönlein purpura or HSP), hematopoietic stem cell transplantation-associated thrombotic microangiopathy (HSCT-TMA), adult respiratory distress syndrome (ARDS,), myocarditis, post-ischemic reperfusion conditions, myocardial infarction, balloon angioplasty, post-pump syndrome in cardiopulmonary bypass or renal bypass, atherosclerosis, hemodialysis, renal ischemia, acute kidney injury mesenteric artery reperfusion after aortic reconstruction, COVID-19, rheumatoid arthritis, osteoarthritis, Spondyloarthropathies, psoriatic arthritis, systemic lupus erythematosus (SLE), lupus nephritis, SLE nephritis, proliferative nephritis, myasthenia gravis, liver fibrosis, hemolytic anemia, tissue regeneration, neural regeneration, dyspnea, hemoptysis, acute respiratory distress syndrome (ARDS), asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, antineutrophil cytoplasmic antibody (ANCA)-associated vasculitides (AAV), Buerger's vasculitis, cryoglobulinemia, Kawasaki disease, Takayasu arteritis, cryoglobulinemia, immune complex-associated inflammation, antiphospholipid syndrome, glomerulonephritis and obesity, immune thrombocytopenia, Cold agglutinin disease, Warm autoimmune hemolytic anemia (wAIHA), thrombotic thrombocytopeni purpura (TTP), abdominal aortic aneurisms, Grave's disease, and hidradenitis suppurativa.
